SEQUENCE LISTING (144 sequences)

SEQ ID NO: 1 - DNA, 76 bp, Synthetic Construct
  source 1..76: mol_type = other DNA
gggccagtggcgcaatggataacgcgtctgactctggatcagaagattccaggttcgactcctggctggctcgcca

SEQ ID NO: 2 - DNA, 76 bp, Synthetic Construct
  source 1..76: mol_type = other DNA
gggccagtggcgcaatggataacgcgtctgactctggatcagaagattctaggttcgactcctggctggctcgcca

SEQ ID NO: 3 - DNA, 76 bp, Synthetic Construct
  source 1..76: mol_type = other DNA
ggccgcgtggcctaatggataaggcgtctgattctggatcagaagattgagggttcgagtcccttcgtggtcgcca

SEQ ID NO: 4 - DNA, 76 bp, Synthetic Construct
  source 1..76: mol_type = other DNA
gacccagtggcctaatggataaggcatcagcctctggagctggggattgtgggttcgagtcccatctgggtcgcca

SEQ ID NO: 5 - DNA, 76 bp, Synthetic Construct
  source 1..76: mol_type = other DNA
gccccagtggcctaatggataaggcactggcctctgaagccagggattgtgggttcgagtcccacctggggtacca

SEQ ID NO: 6 - DNA, 76 bp, Synthetic Construct
  source 1..76: mol_type = other DNA
gccccagtggcctaatggataaggcactggcctctgaagccagggattgtgggttcgagtcccacctggggtgcca

SEQ ID NO: 7 - DNA, 76 bp, Synthetic Construct
  source 1..76: mol_type = other DNA
gccccggtggcctaatggataaggcattggcctctgaagccagggattgtgggttcgagtcccacccggggtacca

SEQ ID NO: 8 - DNA, 76 bp, Synthetic Construct
  source 1..76: mol_type = other DNA
gccccagtggcctaatggataaggcattggcctctgaagccagggattgtgggttcgagtcccatctggggtgcca

SEQ ID NO: 9 - DNA, 76 bp, Synthetic Construct
  source 1..76: mol_type = other DNA
gccccagtggcctgatggataaggtactggcctctgaagccagggattgtgggttcgagttccacctggggtacca

SEQ ID NO: 10 - DNA, 76 bp, Synthetic Construct
  source 1..76: mol_type = other DNA
ggccgcgtggcctaatggataaggcgtctgactctggatcagaagattgcaggttcgagtcctgccgcggtcgcca

SEQ ID NO: 11 - DNA, 76 bp, Synthetic Construct
  source 1..76: mol_type = other DNA
gaccacgtggcctaatggataaggcgtctgactctggatcagaagattgagggttcgaatccctccgtggttacca

SEQ ID NO: 12 - DNA, 76 bp, Synthetic Construct
  source 1..76: mol_type = other DNA
gaccgcgtggcctaatggataaggcgtctgactctggatcagaagattgagggttcgagtcccttcgtggtcgcca

SEQ ID NO: 13 - DNA, 76 bp, Synthetic Construct
  source 1..76: mol_type = other DNA
gaccacgtggcctaatggataaggcgtctgactctggatcagaagattgagggttcgaatcccttcgtggttacca

SEQ ID NO: 14 - DNA, 76 bp, Synthetic Construct
  source 1..76: mol_type = other DNA
gaccacgtggcctaatggataaggcgtctgactctggatcagaagattgagggttcgaatcccttcgtggttgcca

SEQ ID NO: 15 - DNA, 76 bp, Synthetic Construct
  source 1..76: mol_type = other DNA
ggccgtgtggcctaatggataaggcgtctgactctggatcaaaagattgcaggtttgagttctgccacggtcgcca

SEQ ID NO: 16 - DNA, 76 bp, Synthetic Construct
  source 1..76: mol_type = other DNA
ggctccgtggcgcaatggatagcgcattggactctgaattcaaaggttccgggttcgagtcccggcggagtcgcca

SEQ ID NO: 17 - DNA, 76 bp, Synthetic Construct
  source 1..76: mol_type = other DNA
ggctctgtggcgcaatggatagcgcattggactctgaattcaaaggttgtgggttcgaatcccaccagagtcgcca

SEQ ID NO: 18 - DNA, 76 bp, Synthetic Construct
  source 1..76: mol_type = other DNA
ggctctgtggcgcaatggatagcgcattggactctgaattcaaaggttgtgggttcgagtcccaccagagtcgcca

SEQ ID NO: 19 - DNA, 77 bp, Synthetic Construct
  source 1..77: mol_type = other DNA
gtctctgtggcgcaatggacgagcgcgctggactctgaatccagaggttccgggttcgagtcccggcagagatgcca

SEQ ID NO: 20 - DNA, 76 bp, Synthetic Construct
  source 1..76: mol_type = other DNA
ggctctgtggcgcaatggatagcgcattggactctgaattcaaaggttgcgggttcgagtccctccagagtcgcca

SEQ ID NO: 21 - DNA, 88 bp, Synthetic Construct
  source 1..88: mol_type = other DNA
ggctccgtggcgcaatggatagcgcattggactctgagaggctgaaggcattcaaaggttccgggttcgagtcccggcggagtcgcca

SEQ ID NO: 22 - DNA, 91 bp, Synthetic Construct
  source 1..91: mol_type = other DNA
ggctctgtggcgcaatggatagcgcattggactctgagtgacgaatagagcaattcaaaggttgtgggttcgaatcccaccagagtcgcca

SEQ ID NO: 23 - DNA, 94 bp, Synthetic Construct
  source 1..94: mol_type = other DNA
ggctctgtggcgcaatggatagcgcattggactctgagctgagcctagtgtggtcattcaaaggttgtgggttcgagtcccaccagagtcgcca

SEQ ID NO: 24 - DNA, 89 bp, Synthetic Construct
  source 1..89: mol_type = other DNA
ggctctgtggcgcaatggatagcgcattggactctgagatagttagagaaattcaaaggttgtgggttcgagtcccaccagagtcgcca

SEQ ID NO: 25 - DNA, 77 bp, Synthetic Construct; identical to SEQ ID NO: 19
  source 1..77: mol_type = other DNA

SEQ ID NO: 26 - DNA, 90 bp, Synthetic Construct
  source 1..90: mol_type = other DNA
ggctctgtggcgcaatggatagcgcattggactctgagcctaaatcaagagattcaaaggttgcgggttcgagtccctccagagtcgcca

SEQ ID NO: 27 - DNA, 76 bp, Synthetic Construct
  source 1..76: mol_type = other DNA
gggccagtggcgcaatggataacgcgtctgactttggatcagaagattccaggttcgactcctggctggctcgcca

SEQ ID NO: 28 - DNA, 76 bp, Synthetic Construct
  source 1..76: mol_type = other DNA
gggccagtggcgcaatggataacgcgtctgactttggatcagaagattctaggttcgactcctggctggctcgcca

SEQ ID NO: 29 - DNA, 76 bp, Synthetic Construct
  source 1..76: mol_type = other DNA
ggccgcgtggcctaatggataaggcgtctgattttggatcagaagattgagggttcgagtcccttcgtggtcgcca

SEQ ID NO: 30 - DNA, 76 bp, Synthetic Construct
  source 1..76: mol_type = other DNA
gacccagtggcctaatggataaggcatcagcctttggagctggggattgtgggttcgagtcccatctgggtcgcca

SEQ ID NO: 31 - DNA, 76 bp, Synthetic Construct
  source 1..76: mol_type = other DNA
gccccagtggcctaatggataaggcactggcctttgaagccagggattgtgggttcgagtcccacctggggtacca

SEQ ID NO: 32 - DNA, 76 bp, Synthetic Construct
  source 1..76: mol_type = other DNA
gccccagtggcctaatggataaggcactggcctttgaagccagggattgtgggttcgagtcccacctggggtgcca

SEQ ID NO: 33 - DNA, 76 bp, Synthetic Construct
  source 1..76: mol_type = other DNA
gccccggtggcctaatggataaggcattggcctttgaagccagggattgtgggttcgagtcccacccggggtacca

SEQ ID NO: 34 - DNA, 76 bp, Synthetic Construct
  source 1..76: mol_type = other DNA
gccccagtggcctaatggataaggcattggcctttgaagccagggattgtgggttcgagtcccatctggggtgcca

SEQ ID NO: 35 - DNA, 76 bp, Synthetic Construct
  source 1..76: mol_type = other DNA
gccccagtggcctgatggataaggtactggcctttgaagccagggattgtgggttcgagttccacctggggtacca

SEQ ID NO: 36 - DNA, 76 bp, Synthetic Construct
  source 1..76: mol_type = other DNA
ggccgcgtggcctaatggataaggcgtctgactttggatcagaagattgcaggttcgagtcctgccgcggtcgcca

SEQ ID NO: 37 - DNA, 76 bp, Synthetic Construct
  source 1..76: mol_type = other DNA
gaccacgtggcctaatggataaggcgtctgactttggatcagaagattgagggttcgaatccctccgtggttacca

SEQ ID NO: 38 - DNA, 76 bp, Synthetic Construct
  source 1..76: mol_type = other DNA
gaccgcgtggcctaatggataaggcgtctgactttggatcagaagattgagggttcgagtcccttcgtggtcgcca

SEQ ID NO: 39 - DNA, 76 bp, Synthetic Construct
  source 1..76: mol_type = other DNA
gaccacgtggcctaatggataaggcgtctgactttggatcagaagattgagggttcgaatcccttcgtggttacca

SEQ ID NO: 40 - DNA, 76 bp, Synthetic Construct
  source 1..76: mol_type = other DNA
gaccacgtggcctaatggataaggcgtctgactttggatcagaagattgagggttcgaatcccttcgtggttgcca

SEQ ID NO: 41 - DNA, 76 bp, Synthetic Construct
  source 1..76: mol_type = other DNA
ggccgtgtggcctaatggataaggcgtctgactttggatcaaaagattgcaggtttgagttctgccacggtcgcca

SEQ ID NO: 42 - DNA, 76 bp, Synthetic Construct
  source 1..76: mol_type = other DNA
ggctccgtggcgcaatggatagcgcattggactttgaattcaaaggttccgggttcgagtcccggcggagtcgcca

SEQ ID NO: 43 - DNA, 76 bp, Synthetic Construct
  source 1..76: mol_type = other DNA
ggctctgtggcgcaatggatagcgcattggactttgaattcaaaggttgtgggttcgaatcccaccagagtcgcca

SEQ ID NO: 44 - DNA, 76 bp, Synthetic Construct
  source 1..76: mol_type = other DNA
ggctctgtggcgcaatggatagcgcattggactttgaattcaaaggttgtgggttcgagtcccaccagagtcgcca

SEQ ID NO: 45 - DNA, 77 bp, Synthetic Construct
  source 1..77: mol_type = other DNA
gtctctgtggcgcaatggacgagcgcgctggactttgaatccagaggttccgggttcgagtcccggcagagatgcca

SEQ ID NO: 46 - DNA, 76 bp, Synthetic Construct
  source 1..76: mol_type = other DNA
ggctctgtggcgcaatggatagcgcattggactttgaattcaaaggttgcgggttcgagtccctccagagtcgcca

SEQ ID NO: 47 - DNA, 88 bp, Synthetic Construct
  source 1..88: mol_type = other DNA
ggctccgtggcgcaatggatagcgcattggactttgagaggctgaaggcattcaaaggttccgggttcgagtcccggcggagtcgcca

SEQ ID NO: 48 - DNA, 91 bp, Synthetic Construct
  source 1..91: mol_type = other DNA
ggctctgtggcgcaatggatagcgcattggactttgagtgacgaatagagcaattcaaaggttgtgggttcgaatcccaccagagtcgcca

SEQ ID NO: 49 - DNA, 94 bp, Synthetic Construct
  source 1..94: mol_type = other DNA
ggctctgtggcgcaatggatagcgcattggactttgagctgagcctagtgtggtcattcaaaggttgtgggttcgagtcccaccagagtcgcca

SEQ ID NO: 50 - DNA, 89 bp, Synthetic Construct
  source 1..89: mol_type = other DNA
ggctctgtggcgcaatggatagcgcattggactttgagatagttagagaaattcaaaggttgtgggttcgagtcccaccagagtcgcca

SEQ ID NO: 51 - DNA, 77 bp, Synthetic Construct; identical to SEQ ID NO: 45
  source 1..77: mol_type = other DNA

SEQ ID NO: 52 - DNA, 90 bp, Synthetic Construct
  source 1..90: mol_type = other DNA
ggctctgtggcgcaatggatagcgcattggactttgagcctaaatcaagagattcaaaggttgcgggttcgagtccctccagagtcgcca

SEQ ID NO: 53 - DNA, 76 bp, Synthetic Construct
  source 1..76: mol_type = other DNA
gggccagtggcgcaatggataacgcgtctgactgtggatcagaagattccaggttcgactcctggctggctcgcca

SEQ ID NO: 54 - DNA, 76 bp, Synthetic Construct
  source 1..76: mol_type = other DNA
gggccagtggcgcaatggataacgcgtctgactgtggatcagaagattctaggttcgactcctggctggctcgcca

SEQ ID NO: 55 - DNA, 76 bp, Synthetic Construct
  source 1..76: mol_type = other DNA
ggccgcgtggcctaatggataaggcgtctgattgtggatcagaagattgagggttcgagtcccttcgtggtcgcca

SEQ ID NO: 56 - DNA, 76 bp, Synthetic Construct
  source 1..76: mol_type = other DNA
gacccagtggcctaatggataaggcatcagcctgtggagctggggattgtgggttcgagtcccatctgggtcgcca

SEQ ID NO: 57 - DNA, 76 bp, Synthetic Construct
  source 1..76: mol_type = other DNA
gccccagtggcctaatggataaggcactggcctgtgaagccagggattgtgggttcgagtcccacctggggtacca

SEQ ID NO: 58 - DNA, 76 bp, Synthetic Construct
  source 1..76: mol_type = other DNA
gccccagtggcctaatggataaggcactggcctgtgaagccagggattgtgggttcgagtcccacctggggtgcca

SEQ ID NO: 59 - DNA, 76 bp, Synthetic Construct
  source 1..76: mol_type = other DNA
gccccggtggcctaatggataaggcattggcctgtgaagccagggattgtgggttcgagtcccacccggggtacca

SEQ ID NO: 60 - DNA, 76 bp, Synthetic Construct
  source 1..76: mol_type = other DNA
gccccagtggcctaatggataaggcattggcctgtgaagccagggattgtgggttcgagtcccatctggggtgcca

SEQ ID NO: 61 - DNA, 76 bp, Synthetic Construct
  source 1..76: mol_type = other DNA
gccccagtggcctgatggataaggtactggcctgtgaagccagggattgtgggttcgagttccacctggggtacca

SEQ ID NO: 62 - DNA, 76 bp, Synthetic Construct
  source 1..76: mol_type = other DNA
ggccgcgtggcctaatggataaggcgtctgactgtggatcagaagattgcaggttcgagtcctgccgcggtcgcca

SEQ ID NO: 63 - DNA, 76 bp, Synthetic Construct
  source 1..76: mol_type = other DNA
gaccacgtggcctaatggataaggcgtctgactgtggatcagaagattgagggttcgaatccctccgtggttacca

SEQ ID NO: 64 - DNA, 76 bp, Synthetic Construct
  source 1..76: mol_type = other DNA
gaccgcgtggcctaatggataaggcgtctgactgtggatcagaagattgagggttcgagtcccttcgtggtcgcca

SEQ ID NO: 65 - DNA, 76 bp, Synthetic Construct
  source 1..76: mol_type = other DNA
gaccacgtggcctaatggataaggcgtctgactgtggatcagaagattgagggttcgaatcccttcgtggttacca

SEQ ID NO: 66 - DNA, 76 bp, Synthetic Construct
  source 1..76: mol_type = other DNA
gaccacgtggcctaatggataaggcgtctgactgtggatcagaagattgagggttcgaatcccttcgtggttgcca

SEQ ID NO: 67 - DNA, 76 bp, Synthetic Construct
  source 1..76: mol_type = other DNA
ggccgtgtggcctaatggataaggcgtctgactgtggatcaaaagattgcaggtttgagttctgccacggtcgcca

SEQ ID NO: 68 - DNA, 76 bp, Synthetic Construct
  source 1..76: mol_type = other DNA
ggctccgtggcgcaatggatagcgcattggactgtgaattcaaaggttccgggttcgagtcccggcggagtcgcca

SEQ ID NO: 69 - DNA, 76 bp, Synthetic Construct
  source 1..76: mol_type = other DNA
ggctctgtggcgcaatggatagcgcattggactgtgaattcaaaggttgtgggttcgaatcccaccagagtcgcca

SEQ ID NO: 70 - DNA, 76 bp, Synthetic Construct
  source 1..76: mol_type = other DNA
ggctctgtggcgcaatggatagcgcattggactgtgaattcaaaggttgtgggttcgagtcccaccagagtcgcca

SEQ ID NO: 71 - DNA, 77 bp, Synthetic Construct
  source 1..77: mol_type = other DNA
gtctctgtggcgcaatggacgagcgcgctggactgtgaatccagaggttccgggttcgagtcccggcagagatgcca

SEQ ID NO: 72 - DNA, 76 bp, Synthetic Construct
  source 1..76: mol_type = other DNA
ggctctgtggcgcaatggatagcgcattggactgtgaattcaaaggttgcgggttcgagtccctccagagtcgcca

SEQ ID NO: 73 - DNA, 88 bp, Synthetic Construct
  source 1..88: mol_type = other DNA
ggctccgtggcgcaatggatagcgcattggactgtgagaggctgaaggcattcaaaggttccgggttcgagtcccggcggagtcgcca

SEQ ID NO: 74 - DNA, 91 bp, Synthetic Construct
  source 1..91: mol_type = other DNA
ggctctgtggcgcaatggatagcgcattggactgtgagtgacgaatagagcaattcaaaggttgtgggttcgaatcccaccagagtcgcca

SEQ ID NO: 75 - DNA, 94 bp, Synthetic Construct
  source 1..94: mol_type = other DNA
ggctctgtggcgcaatggatagcgcattggactgtgagctgagcctagtgtggtcattcaaaggttgtgggttcgagtcccaccagagtcgcca

SEQ ID NO: 76 - DNA, 89 bp, Synthetic Construct
  source 1..89: mol_type = other DNA
ggctctgtggcgcaatggatagcgcattggactgtgagatagttagagaaattcaaaggttgtgggttcgagtcccaccagagtcgcca

SEQ ID NO: 77 - DNA, 77 bp, Synthetic Construct; identical to SEQ ID NO: 71
  source 1..77: mol_type = other DNA

SEQ ID NO: 78 - DNA, 90 bp, Synthetic Construct
  source 1..90: mol_type = other DNA
ggctctgtggcgcaatggatagcgcattggactgtgagcctaaatcaagagattcaaaggttgcgggttcgagtccctccagagtcgcca

SEQ ID NO: 79 - DNA, 76 bp, Synthetic Construct
  source 1..76: mol_type = other DNA
gggccagtggcgcaatggataacgcgtctgactccagatcagaagattccaggttcgactcctggctggctcgcca

SEQ ID NO: 80 - DNA, 76 bp, Synthetic Construct
  source 1..76: mol_type = other DNA
gggccagtggcgcaatggataacgcgtctgactccagatcagaagattctaggttcgactcctggctggctcgcca

SEQ ID NO: 81 - DNA, 76 bp, Synthetic Construct
  source 1..76: mol_type = other DNA
ggccgcgtggcctaatggataaggcgtctgattccagatcagaagattgagggttcgagtcccttcgtggtcgcca

SEQ ID NO: 82 - DNA, 76 bp, Synthetic Construct
  source 1..76: mol_type = other DNA
gacccagtggcctaatggataaggcatcagcctccagagctggggattgtgggttcgagtcccatctgggtcgcca

SEQ ID NO: 83 - DNA, 76 bp, Synthetic Construct
  source 1..76: mol_type = other DNA
gccccagtggcctaatggataaggcactggcctccaaagccagggattgtgggttcgagtcccacctggggtacca

SEQ ID NO: 84 - DNA, 76 bp, Synthetic Construct
  source 1..76: mol_type = other DNA
gccccagtggcctaatggataaggcactggcctccaaagccagggattgtgggttcgagtcccacctggggtgcca

SEQ ID NO: 85 - DNA, 76 bp, Synthetic Construct
  source 1..76: mol_type = other DNA
gccccggtggcctaatggataaggcattggcctccaaagccagggattgtgggttcgagtcccacccggggtacca

SEQ ID NO: 86 - DNA, 76 bp, Synthetic Construct
  source 1..76: mol_type = other DNA
gccccagtggcctaatggataaggcattggcctccaaagccagggattgtgggttcgagtcccatctggggtgcca

SEQ ID NO: 87 - DNA, 76 bp, Synthetic Construct
  source 1..76: mol_type = other DNA
gccccagtggcctgatggataaggtactggcctccaaagccagggattgtgggttcgagttccacctggggtacca

SEQ ID NO: 88 - DNA, 76 bp, Synthetic Construct
  source 1..76: mol_type = other DNA
ggccgcgtggcctaatggataaggcgtctgactccagatcagaagattgcaggttcgagtcctgccgcggtcgcca

SEQ ID NO: 89 - DNA, 76 bp, Synthetic Construct
  source 1..76: mol_type = other DNA
gaccacgtggcctaatggataaggcgtctgactccagatcagaagattgagggttcgaatccctccgtggttacca

SEQ ID NO: 90 - DNA, 76 bp, Synthetic Construct
  source 1..76: mol_type = other DNA
gaccgcgtggcctaatggataaggcgtctgactccagatcagaagattgagggttcgagtcccttcgtggtcgcca

SEQ ID NO: 91 - DNA, 76 bp, Synthetic Construct
  source 1..76: mol_type = other DNA
gaccacgtggcctaatggataaggcgtctgactccagatcagaagattgagggttcgaatcccttcgtggttacca

SEQ ID NO: 92 - DNA, 76 bp, Synthetic Construct
  source 1..76: mol_type = other DNA
gaccacgtggcctaatggataaggcgtctgactccagatcagaagattgagggttcgaatcccttcgtggttgcca

SEQ ID NO: 93 - DNA, 76 bp, Synthetic Construct
  source 1..76: mol_type = other DNA
ggccgtgtggcctaatggataaggcgtctgactccagatcaaaagattgcaggtttgagttctgccacggtcgcca

SEQ ID NO: 94 - DNA, 76 bp, Synthetic Construct
  source 1..76: mol_type = other DNA
ggctccgtggcgcaatggatagcgcattggactccaaattcaaaggttccgggttcgagtcccggcggagtcgcca

SEQ ID NO: 95 - DNA, 76 bp, Synthetic Construct
  source 1..76: mol_type = other DNA
ggctctgtggcgcaatggatagcgcattggactccaaattcaaaggttgtgggttcgaatcccaccagagtcgcca

SEQ ID NO: 96 - DNA, 76 bp, Synthetic Construct
  source 1..76: mol_type = other DNA
ggctctgtggcgcaatggatagcgcattggactccaaattcaaaggttgtgggttcgagtcccaccagagtcgcca

SEQ ID NO: 97 - DNA, 77 bp, Synthetic Construct
  source 1..77: mol_type = other DNA
gtctctgtggcgcaatggacgagcgcgctggactccaaatccagaggttccgggttcgagtcccggcagagatgcca

SEQ ID NO: 98 - DNA, 76 bp, Synthetic Construct
  source 1..76: mol_type = other DNA
ggctctgtggcgcaatggatagcgcattggactccaaattcaaaggttgcgggttcgagtccctccagagtcgcca

SEQ ID NO: 99 - DNA, 88 bp, Synthetic Construct
  source 1..88: mol_type = other DNA
ggctccgtggcgcaatggatagcgcattggactccaagaggctgaaggcattcaaaggttccgggttcgagtcccggcggagtcgcca

SEQ ID NO: 100 - DNA, 91 bp, Synthetic Construct
  source 1..91: mol_type = other DNA
ggctctgtggcgcaatggatagcgcattggactccaagtgacgaatagagcaattcaaaggttgtgggttcgaatcccaccagagtcgcca

SEQ ID NO: 101 - DNA, 94 bp, Synthetic Construct
  source 1..94: mol_type = other DNA
ggctctgtggcgcaatggatagcgcattggactccaagctgagcctagtgtggtcattcaaaggttgtgggttcgagtcccaccagagtcgcca

SEQ ID NO: 102 - DNA, 89 bp, Synthetic Construct
  source 1..89: mol_type = other DNA
ggctctgtggcgcaatggatagcgcattggactccaagatagttagagaaattcaaaggttgtgggttcgagtcccaccagagtcgcca

SEQ ID NO: 103 - DNA, 77 bp, Synthetic Construct; identical to SEQ ID NO: 97
  source 1..77: mol_type = other DNA

SEQ ID NO: 104 - DNA, 90 bp, Synthetic Construct
  source 1..90: mol_type = other DNA
ggctctgtggcgcaatggatagcgcattggactccaagcctaaatcaagagattcaaaggttgcgggttcgagtccctccagagtcgcca

SEQ ID NO: 105 - DNA, 76 bp, Synthetic Construct
  source 1..76: mol_type = other DNA
gggccagtggcgcaatggataacgcgtctgactgcagatcagaagattccaggttcgactcctggctggctcgcca

SEQ ID NO: 106 - DNA, 76 bp, Synthetic Construct
  source 1..76: mol_type = other DNA
gggccagtggcgcaatggataacgcgtctgactgcagatcagaagattctaggttcgactcctggctggctcgcca

SEQ ID NO: 107 - DNA, 76 bp, Synthetic Construct
  source 1..76: mol_type = other DNA
ggccgcgtggcctaatggataaggcgtctgattgcagatcagaagattgagggttcgagtcccttcgtggtcgcca

SEQ ID NO: 108 - DNA, 76 bp, Synthetic Construct
  source 1..76: mol_type = other DNA
gacccagtggcctaatggataaggcatcagcctgcagagctggggattgtgggttcgagtcccatctgggtcgcca

SEQ ID NO: 109 - DNA, 76 bp, Synthetic Construct
  source 1..76: mol_type = other DNA
gccccagtggcctaatggataaggcactggcctgcaaagccagggattgtgggttcgagtcccacctggggtacca

SEQ ID NO: 110 - DNA, 76 bp, Synthetic Construct
  source 1..76: mol_type = other DNA
gccccagtggcctaatggataaggcactggcctgcaaagccagggattgtgggttcgagtcccacctggggtgcca

SEQ ID NO: 111 - DNA, 76 bp, Synthetic Construct
  source 1..76: mol_type = other DNA
gccccggtggcctaatggataaggcattggcctgcaaagccagggattgtgggttcgagtcccacccggggtacca

SEQ ID NO: 112 - DNA, 76 bp, Synthetic Construct
  source 1..76: mol_type = other DNA
gccccagtggcctaatggataaggcattggcctgcaaagccagggattgtgggttcgagtcccatctggggtgcca

SEQ ID NO: 113 - DNA, 76 bp, Synthetic Construct
  source 1..76: mol_type = other DNA
gccccagtggcctgatggataaggtactggcctgcaaagccagggattgtgggttcgagttccacctggggtacca

SEQ ID NO: 114 - DNA, 76 bp, Synthetic Construct
  source 1..76: mol_type = other DNA
ggccgcgtggcctaatggataaggcgtctgactgcagatcagaagattgcaggttcgagtcctgccgcggtcgcca

SEQ ID NO: 115 - DNA, 76 bp, Synthetic Construct
  source 1..76: mol_type = other DNA
gaccacgtggcctaatggataaggcgtctgactgcagatcagaagattgagggttcgaatccctccgtggttacca

SEQ ID NO: 116 - DNA, 76 bp, Synthetic Construct
  source 1..76: mol_type = other DNA
gaccgcgtggcctaatggataaggcgtctgactgcagatcagaagattgagggttcgagtcccttcgtggtcgcca

SEQ ID NO: 117 - DNA, 76 bp, Synthetic Construct
  source 1..76: mol_type = other DNA
gaccacgtggcctaatggataaggcgtctgactgcagatcagaagattgagggttcgaatcccttcgtggttacca

SEQ ID NO: 118 - DNA, 76 bp, Synthetic Construct
  source 1..76: mol_type = other DNA
gaccacgtggcctaatggataaggcgtctgactgcagatcagaagattgagggttcgaatcccttcgtggttgcca

SEQ ID NO: 119 - DNA, 76 bp, Synthetic Construct
  source 1..76: mol_type = other DNA
ggccgtgtggcctaatggataaggcgtctgactgcagatcaaaagattgcaggtttgagttctgccacggtcgcca

SEQ ID NO: 120 - DNA, 76 bp, Synthetic Construct
  source 1..76: mol_type = other DNA
ggctccgtggcgcaatggatagcgcattggactgcaaattcaaaggttccgggttcgagtcccggcggagtcgcca

SEQ ID NO: 121 - DNA, 76 bp, Synthetic Construct
  source 1..76: mol_type = other DNA
ggctctgtggcgcaatggatagcgcattggactgcaaattcaaaggttgtgggttcgaatcccaccagagtcgcca

SEQ ID NO: 122 - DNA, 76 bp, Synthetic Construct
  source 1..76: mol_type = other DNA
ggctctgtggcgcaatggatagcgcattggactgcaaattcaaaggttgtgggttcgagtcccaccagagtcgcca

SEQ ID NO: 123 - DNA, 77 bp, Synthetic Construct
  source 1..77: mol_type = other DNA
gtctctgtggcgcaatggacgagcgcgctggactgcaaatccagaggttccgggttcgagtcccggcagagatgcca

SEQ ID NO: 124 - DNA, 76 bp, Synthetic Construct
  source 1..76: mol_type = other DNA
ggctctgtggcgcaatggatagcgcattggactgcaaattcaaaggttgcgggttcgagtccctccagagtcgcca

SEQ ID NO: 125 - DNA, 88 bp, Synthetic Construct
  source 1..88: mol_type = other DNA
ggctccgtggcgcaatggatagcgcattggactgcaagaggctgaaggcattcaaaggttccgggttcgagtcccggcggagtcgcca

SEQ ID NO: 126 - DNA, 91 bp, Synthetic Construct
  source 1..91: mol_type = other DNA
ggctctgtggcgcaatggatagcgcattggactgcaagtgacgaatagagcaattcaaaggttgtgggttcgaatcccaccagagtcgcca

SEQ ID NO: 127 - DNA, 94 bp, Synthetic Construct
  source 1..94: mol_type = other DNA
ggctctgtggcgcaatggatagcgcattggactgcaagctgagcctagtgtggtcattcaaaggttgtgggttcgagtcccaccagagtcgcca

SEQ ID NO: 128 - DNA, 89 bp, Synthetic Construct
  source 1..89: mol_type = other DNA
ggctctgtggcgcaatggatagcgcattggactgcaagatagttagagaaattcaaaggttgtgggttcgagtcccaccagagtcgcca

SEQ ID NO: 129 - DNA, 77 bp, Synthetic Construct; identical to SEQ ID NO: 123
  source 1..77: mol_type = other DNA

SEQ ID NO: 130 - DNA, 90 bp, Synthetic Construct
  source 1..90: mol_type = other DNA
ggctctgtggcgcaatggatagcgcattggactgcaagcctaaatcaagagattcaaaggttgcgggttcgagtccctccagagtcgcca

SEQ ID NO: 131 - DNA, 85 bp, Synthetic Construct
  source 1..85: mol_type = other DNA
gtagtcgtggccgagtggttaaggcgatggactccgaatccattggggtttccccgcgcaggttcgaatcctgccgactacgcca

SEQ ID NO: 132 - DNA, 85 bp, Synthetic Construct
  source 1..85: mol_type = other DNA
gctgtgatggccgagtggttaaggcgttggactccgaatccaatggggtctccccgcgcaggttcgaatcctgctcacagcgcca

SEQ ID NO: 133 - DNA, 88 bp, Synthetic Construct
  source 1..88: mol_type = other DNA
gctgtgatggccgagtggttaaggcgttggactccgaatccaatggggtccactccccgcgcaggttcgaatcctgctcacagcgcca

SEQ ID NO: 134 - DNA, 85 bp, Synthetic Construct
  source 1..85: mol_type = other DNA
gctgtgatggccgagtggttaaggcgttggactcgaaatccaatggggtctccccgcgcaggttcgaatcctgctcacagcgcca

SEQ ID NO: 135 - DNA, 6675 bp, Synthetic Construct
  source 1..6675: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggtgtctaagggcgaggaactgttcaccggcgtggtgcctatcctggtggaactggacggagatgtgaacggccataagtttagcgtgtccggcgaaggcgaaggcgacgccacctacggcaagctgaccctgaagttcatctgtaccaccggcaaactgcccgtgccatggcctaccctggtgacaacactgacctatggcgttcagtgcttcagcagataccccgaccacatgaaacagcacgatttctttaaaagcgccatgcctgagggctacgtgcaggagagaacaatcttcttcaaagacgacggaaattacaagaccagagccgaggtgaagttcgagggcgatacactggtcaatagaatcgagctgaagggaatcgactttaaggaagatggcaacatcctgggccacaagctcgagtacaactacaacagccacaacgtgtacatcatggctgataagcagaagaacggcatcaaggtgaatttcaagatccggcacaacatcgaggacggcagcgtgcaactggccgaccactaccagcagaacacccctattggagatggccctgtgctgctgcctgacaaccactacctgtctacacagagcaaactgtccaaggaccccaacgagaagcgggaccacatggtcctgctggaattcgtgaccgccgctggcatcacactgggaatggacgagctgtacaagggcggcagcggcggatctatggtgtccaagggcgaggaagataatatggccatcatcaaggaattcatgcggttcaaagtgcacatggaaggcagcgtgaacggccatgagtttgagatcgagggagaaggcgagggtagaccttacgagggcacccagaccgccaagctgaaggtgacaaagggcggccctctgccattcgcctgggacattctgagccctcagttcatgtacggctctaaggcttatgtgaagcaccccgctgatatccctgactatctgaagctctccttccccgagggcttcaagtgggaacgggtgatgaacttcgaggatggcggagtggtgacagtgacacaagacagcagcctgcaggacggcgaattcatctacaaagtgaagctgagaggcaccaattttccatctgatggacctgtgatgcagaaaaagaccatgggctgggaggccagcagcgagagaatgtaccctgaggacggagctctgaagggcgaaatcaaacagagactgaagctgaaggacggaggccactacgacgccgaagtgaaaacaacctacaaggccaagaagcccgtccagctgcctggcgcctacaacgttaacatcaagctggacatcaccagccacaacgaggattacaccatcgtggaacagtacgagcgcgccgaaggaagacacagcaccggcggcatggacgagctgtacaagtgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 136 - DNA, 6675 bp, Synthetic Construct
  source 1..6675: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggtgtctaagggcgaggaactgttcaccggcgtggtgcctatcctggtggaactggacggagatgtgaacggccataagtttagcgtgtccggcgaaggcgaaggcgacgccacctacggcaagctgaccctgaagttcatctgtaccaccggcaaactgcccgtgccatggcctaccctggtgacaacactgacctatggcgttcagtgcttcagcagataccccgaccacatgaaacagcacgatttctttaaaagcgccatgcctgagggctacgtgcaggagcagacaatcttcttcaaagacgacggaaattacaagaccagagccgaggtgaagttcgagggcgatacactggtcaatagaatcgagctgaagggaatcgactttaaggaagatggcaacatcctgggccacaagctcgagtacaactacaacagccacaacgtgtacatcatggctgataagcagaagaacggcatcaaggtgaatttcaagatccggcacaacatcgaggacggcagcgtgcaactggccgaccactaccagcagaacacccctattggagatggccctgtgctgctgcctgacaaccactacctgtctacacagagcaaactgtccaaggaccccaacgagaagcgggaccacatggtcctgctggaattcgtgaccgccgctggcatcacactgggaatggacgagctgtacaagggcggcagcggcggatctatggtgtccaagggcgaggaagataatatggccatcatcaaggaattcatgcggttcaaagtgcacatggaaggcagcgtgaacggccatgagtttgagatcgagggagaaggcgagggtagaccttacgagggcacccagaccgccaagctgaaggtgacaaagggcggccctctgccattcgcctgggacattctgagccctcagttcatgtacggctctaaggcttatgtgaagcaccccgctgatatccctgactatctgaagctctccttccccgagggcttcaagtgggaacgggtgatgaacttcgaggatggcggagtggtgacagtgacacaagacagcagcctgcaggacggcgaattcatctacaaagtgaagctgagaggcaccaattttccatctgatggacctgtgatgcagaaaaagaccatgggctgggaggccagcagcgagagaatgtaccctgaggacggagctctgaagggcgaaatcaaacagagactgaagctgaaggacggaggccactacgacgccgaagtgaaaacaacctacaaggccaagaagcccgtccagctgcctggcgcctacaacgttaacatcaagctggacatcaccagccacaacgaggattacaccatcgtggaacagtacgagcgcgccgaaggaagacacagcaccggcggcatggacgagctgtacaagtgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 137 - DNA, 7149 bp, Synthetic Construct
  source 1..7149: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggtgtctaagggcgaggaactgttcaccggcgtggtgcctatcctggtggaactggacggagatgtgaacggccataagtttagcgtgtccggcgaaggcgaaggcgacgccacctacggcaagctgaccctgaagttcatctgtaccaccggcaaactgcccgtgccatggcctaccctggtgacaacactgacctatggcgttcagtgcttcagcagataccccgaccacatgaaacagcacgatttctttaaaagcgccatgcctgagggctacgtgcaggagcagacaatcttcttcaaagacgacggaaattacaagaccagagccgaggtgaagttcgagggcgatacactggtcaatagaatcgagctgaagggaatcgactttaaggaagatggcaacatcctgggccacaagctcgagtacaactacaacagccacaacgtgtacatcatggctgataagcagaagaacggcatcaaggtgaatttcaagatccggcacaacatcgaggacggcagcgtgcaactggccgaccactaccagcagaacacccctattggagatggccctgtgctgctgcctgacaaccactacctgtctacacagagcaaactgtccaaggaccccaacgagaagcgggaccacatggtcctgctggaattcgtgaccgccgctggcatcacactgggaatggacgagctgtacaagggcggcagcggcggatctatggtgtccaagggcgaggaagataatatggccatcatcaaggaattcatgcggttcaaagtgcacatggaaggcagcgtgaacggccatgagtttgagatcgagggagaaggcgagggtagaccttacgagggcacccagaccgccaagctgaaggtgacaaagggcggccctctgccattcgcctgggacattctgagccctcagttcatgtacggctctaaggcttatgtgaagcaccccgctgatatccctgactatctgaagctctccttccccgagggcttcaagtgggaacgggtgatgaacttcgaggatggcggagtggtgacagtgacacaagacagcagcctgcaggacggcgaattcatctacaaagtgaagctgagaggcaccaattttccatctgatggacctgtgatgcagaaaaagaccatgggctgggaggccagcagcgagagaatgtaccctgaggacggagctctgaagggcgaaatcaaacagagactgaagctgaaggacggaggccactacgacgccgaagtgaaaacaacctacaaggccaagaagcccgtccagctgcctggcgcctacaacgttaacatcaagctggacatcaccagccacaacgaggattacaccatcgtggaacagtacgagcgcgccgaaggaagacacagcaccggcggcatggacgagctgtacaagtgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttcccatcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctctgaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaatttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 138 - DNA, 6675 bp, Synthetic Construct
  source 1..6675: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggtgtctaagggcgaggaactgttcaccggcgtggtgcctatcctggtggaactggacggagatgtgaacggccataagtttagcgtgtccggcgaaggcgaaggcgacgccacctacggcaagctgaccctgaagttcatctgtaccaccggcaaactgcccgtgccatggcctaccctggtgacaacactgacctatggcgttcagtgcttcagcagataccccgaccacatgaaacagcacgatttctttaaaagcgccatgcctgagggctacgtgcaggagtgcacaatcttcttcaaagacgacggaaattacaagaccagagccgaggtgaagttcgagggcgatacactggtcaatagaatcgagctgaagggaatcgactttaaggaagatggcaacatcctgggccacaagctcgagtacaactacaacagccacaacgtgtacatcatggctgataagcagaagaacggcatcaaggtgaatttcaagatccggcacaacatcgaggacggcagcgtgcaactggccgaccactaccagcagaacacccctattggagatggccctgtgctgctgcctgacaaccactacctgtctacacagagcaaactgtccaaggaccccaacgagaagcgggaccacatggtcctgctggaattcgtgaccgccgctggcatcacactgggaatggacgagctgtacaagggcggcagcggcggatctatggtgtccaagggcgaggaagataatatggccatcatcaaggaattcatgcggttcaaagtgcacatggaaggcagcgtgaacggccatgagtttgagatcgagggagaaggcgagggtagaccttacgagggcacccagaccgccaagctgaaggtgacaaagggcggccctctgccattcgcctgggacattctgagccctcagttcatgtacggctctaaggcttatgtgaagcaccccgctgatatccctgactatctgaagctctccttccccgagggcttcaagtgggaacgggtgatgaacttcgaggatggcggagtggtgacagtgacacaagacagcagcctgcaggacggcgaattcatctacaaagtgaagctgagaggcaccaattttccatctgatggacctgtgatgcagaaaaagaccatgggctgggaggccagcagcgagagaatgtaccctgaggacggagctctgaagggcgaaatcaaacagagactgaagctgaaggacggaggccactacgacgccgaagtgaaaacaacctacaaggccaagaagcccgtccagctgcctggcgcctacaacgttaacatcaagctggacatcaccagccacaacgaggattacaccatcgtggaacagtacgagcgcgccgaaggaagacacagcaccggcggcatggacgagctgtacaagtgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 139 - DNA, 7149 bp, Synthetic Construct
  source 1..7149: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggtgtctaagggcgaggaactgttcaccggcgtggtgcctatcctggtggaactggacggagatgtgaacggccataagtttagcgtgtccggcgaaggcgaaggcgacgccacctacggcaagctgaccctgaagttcatctgtaccaccggcaaactgcccgtgccatggcctaccctggtgacaacactgacctatggcgttcagtgcttcagcagataccccgaccacatgaaacagcacgatttctttaaaagcgccatgcctgagggctacgtgcaggagtgcacaatcttcttcaaagacgacggaaattacaagaccagagccgaggtgaagttcgagggcgatacactggtcaatagaatcgagctgaagggaatcgactttaaggaagatggcaacatcctgggccacaagctcgagtacaactacaacagccacaacgtgtacatcatggctgataagcagaagaacggcatcaaggtgaatttcaagatccggcacaacatcgaggacggcagcgtgcaactggccgaccactaccagcagaacacccctattggagatggccctgtgctgctgcctgacaaccactacctgtctacacagagcaaactgtccaaggaccccaacgagaagcgggaccacatggtcctgctggaattcgtgaccgccgctggcatcacactgggaatggacgagctgtacaagggcggcagcggcggatctatggtgtccaagggcgaggaagataatatggccatcatcaaggaattcatgcggttcaaagtgcacatggaaggcagcgtgaacggccatgagtttgagatcgagggagaaggcgagggtagaccttacgagggcacccagaccgccaagctgaaggtgacaaagggcggccctctgccattcgcctgggacattctgagccctcagttcatgtacggctctaaggcttatgtgaagcaccccgctgatatccctgactatctgaagctctccttccccgagggcttcaagtgggaacgggtgatgaacttcgaggatggcggagtggtgacagtgacacaagacagcagcctgcaggacggcgaattcatctacaaagtgaagctgagaggcaccaattttccatctgatggacctgtgatgcagaaaaagaccatgggctgggaggccagcagcgagagaatgtaccctgaggacggagctctgaagggcgaaatcaaacagagactgaagctgaaggacggaggccactacgacgccgaagtgaaaacaacctacaaggccaagaagcccgtccagctgcctggcgcctacaacgttaacatcaagctggacatcaccagccacaacgaggattacaccatcgtggaacagtacgagcgcgccgaaggaagacacagcaccggcggcatggacgagctgtacaagtgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttcccatcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctgcaaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaatttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 140 - DNA, 482 bp, Synthetic Construct
  source 1..482: mol_type = other DNA
ggagaataggaaggctgccgctccaatggggctgctgacgtctgctggagggaaggcatggtctgcgccaccgagcagaggcgcacgagcgcagacgtctctaggcggcgcgggtggagccgggcggagccgggcggagccggctggaggcgggcgtgttgataggcagctatagttgcacagggccgacagcgcacgcggctgtgatggccgagtggttaaggcgttggactccgaatccaatggggtctccccgcgcaggttcgaatcctgctcacagcgtcactagttttggtatttaggtatctcggtctctggagaaggaaaaagcagtttgttttctggttctttccttggagaatctcccgcgttattctgagaatctcagagactcggccgtcagggaccagcccgcgagcgccctcttgcgattctcatagtcctatcgacctgcaaaaacttttttactgcggatttcaatt

SEQ ID NO: 141 - DNA, 473 bp, Synthetic Construct
  source 1..473: mol_type = other DNA
atcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctgcaaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaattt

SEQ ID NO: 142 - DNA, 473 bp, Synthetic Construct
  source 1..473: mol_type = other DNA
atcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctgtgaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaattt

SEQ ID NO: 143 - DNA, 473 bp, Synthetic Construct
  source 1..473: mol_type = other DNA
atcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctctgaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaattt

SEQ ID NO: 144 - DNA, 473 bp, Synthetic construct
  source 1..473: mol_type = other DNA
atcggtgcagtttcgagggtaaagcctttggcgcggtgatgtggacttttgttctctaactacaactcccagcatacgtcacccctcacgtgggcgctaggtgtggtttcgtgggatagggtaaggcgaaaatgaagtgacgatgacgtaccaaagaggaaaatagttgaatatgttgtgtgagcgcgtcgtttgcagaagccccagtggcctaatggataaggcattggcctccaaagccagggattgtgggttcgagtcccatctggggtggcctgtgacttttgtccttttttcccctttctaaacaaagcgaaatatgccaactttactttttttttttttaaatgggaagtgtgtgtgtgtttttactagctggtgggctgcaattttcgagaatctgcacacacccatgtcctttgtctgagccgcaggtcccctgggatttggggagacgtcaatacgttgaattt

(19) United States
(12) Patent Application Publication
(10) Pub. No.: US 2026/0248838 A1
(43) Pub. Date: Aug. 27, 2026

(54) ENGINEERED tRNA AND METHODS OF USE

(71) Applicant: THE UNIVERSITY OF CHICAGO, Chicago, IL (US)

(72) Inventors: Tao PAN, Chicago, IL (US); Christopher KATANSKI, Chicago, IL (US); Yichen HOU, Chicago, IL (US); Wen ZHANG, Chicago, IL (US)

(73) Assignee: THE UNIVERSITY OF CHICAGO, Chicago, IL (US)

(21) Appl. No.: 18/877,939

(22) PCT Filed: Jun. 22, 2023
(86) PCT No.: PCT/US2023/068918

§ 371 Date: Dec. 20, 2024

Related U.S. Application Data
(60) Provisional application No. 63/354,364, filed on Jun. 22, 2022.
(60) Provisional application No. 63/438,236, filed on Jan. 10, 2023.

(51) Int. Cl. A61K 31/7105; A61K 48/00; C12N 5/071; C12N 15/864

(52) U.S. Cl. CPC A61K 31/7105; A61K 48/005; C12N 5/0602; C12N 15/8645; C12N 2320/34; C12N 2750/14132; C12N 2750/14143

(57) ABSTRACT

In general, the current disclosure relates to the tRNAs that encode for one amino acid but are covalently linked to a different amino acid. The tRNAs can correct missense mutations by providing a different amino acid during protein synthesis. Such tRNAs can be used to correct disease-causing missense mutations.

This application claims the benefit of priority to U.S. Provisional Patent Application Ser. No. 63/354,364 filed Jun. 22, 2022 and U.S. Provisional Patent Application Ser. No. 63/438,236 filed Jan. 10, 2023, each of which are hereby incorporated by reference in their entirety.

This invention was made with government support under grant number GM105386 awarded by the National Institutes of Health. The government has certain rights in the invention.

### BACKGROUND OF THE INVENTION

### I. Sequence Listing

The instant application contains a Sequence Listing which has been submitted in ASCII format and is hereby incorporated by reference in its entirety. Said ASCII copy, created on Jun. 21, 2023, is named ARCD.P0781WO—Sequence Listing.txt and is 172,211 bytes in size.

### II. Field of the Invention

This invention relates to the field of molecular biology, genetic engineering, and medicine.

### III. Background

Mutation in protein-coding regions of DNA can result in changing the amino acid identity in the protein product (missense) or introduction of a premature stop codon (nonsense). Several patents and companies have recently embarked on treating nonsense diseases with engineered tRNAs that read through a stop codon—thus bypassing the nonsense mutation (PMID: 30778053, 33567469). Similar principles can be applied to missense mutations.

The concept underlying MC-tRNAs has previously been described as missense suppressor tRNA-related to more commonly discussed [nonsense] suppressor tRNAs. These altered tRNAs have been shown be functional in the bacteria and yeast (ISBM: 978-3-642-75178-3. PMID: 2502189, PMID: 30007351), and occur at low levels in the human population (PMID: 30643023), though their expression is unclear. In yeast such tRNAs have been described through selection experiments to restore mutant protein function and it was suggested that such ideas could be applied to disease (PMID: 32476470). A fundamental obstacle to execution of this notion has to do with proofreading of tRNAs by their cognate amino-acyl synthetases. The fidelity of the genetic code is ensured by faithful charging of a set of isoaccepting tRNAs by their cognate synthetase—deviations from this lead to proteome wide mistranslation with both deleterious and adaptive effects. To ensure faithful charging, synthetases structurally check many “identity elements” of tRNAs to ensure accuracy. With the exception of type II tRNAs, tRNA-Ser and tRNA-Leu, the anticodon of a tRNA is considered an identity element for synthetase proofreading (PMID: 4879401, 8128220). Thus it is reasonable to expect that mutation of the anticodon of a tRNA, as with MC-tRNAs, will result in rejection by the cognate synthetase and a translation incompetent tRNAs (PMID 11698642, 28660466, 9801296). Recently, it has been shown that different isodecoders of tRNA-Arg and others have different efficacy and nonsense suppression (PMID: 30778053). This suggests that these tRNAs can tolerate mutation to the anticodon loop and remain translation competent, indicating successful amino-acylation at some level. It is unclear if these tRNAs can be engineered to translate non-stop codons, however. Further, it is not clear the level or identity of amino-acylation of these tRNAs.

Of particular interest are MC-tRNAs that natively deliver arginine to treat genetic diseases that are derived from missense mutation of Arg residues. It was found that among known pathogenic single base mutations, mutations of the Arg codons to several other amino acids are the most prevalent.

### SUMMARY OF THE INVENTION

In general, the current disclosure relates to the tRNAs that correct missense mutations using tRNAs that encode for one amino acid but provide a different amino acid during protein synthesis. Such tRNAs can be used to correct missense mutations, including those that cause or contribute to disease.

Disclosed herein are tRNA molecules covalently linked to a first amino acid where the tRNA molecule comprises an anticodon loop sequence capable of hybridizing with an mRNA sequence that encodes for a second amino acid that is different from the first amino acid.

The first amino acid can be any amino acid, including any of Ala, Arg, Asn, Asp, Cys, Glu, Gln, Gly, His, Ile, Leu, Lys, Met, Phe, Pro, Ser, Thr, Trp, Tyr, or Val. In some aspects, the first amino acid is Ala. In some aspects, the first amino acid is Arg. In some aspects, the first amino acid is Asn. In some aspects, the first amino acid is Asp. In some aspects, the first amino acid is Cys. In some aspects, the first amino acid is Glu. In some aspects, the first amino acid is Gln. In some aspects, the first amino acid is Gly. In some aspects, the first amino acid is His. In some aspects, the first amino acid is Ile. In some aspects, the first amino acid is Leu. In some aspects, the first amino acid is Lys. In some aspects, the first amino acid is Met. In some aspects, the first amino acid is Phe. In some aspects. In some aspects, the first amino acid is the first amino acid is Pro. In some aspects. In some aspects, the first amino acid is the first amino acid is Ser. In some aspects, the first amino acid is Thr. In some aspects, the first amino acid is Trp. In some aspects, the first amino acid is Tyr. In some aspects, the first amino acid is Val.

In some aspects, the first amino acid is not Ala. In some aspects, the first amino acid is not Arg. In some aspects, the first amino acid is not Asn. In some aspects, the first amino acid is not Asp. In some aspects, the first amino acid is not Cys. In some aspects, the first amino acid is not Glu. In some aspects, the first amino acid is not Gln. In some aspects, the first amino acid is not Gly. In some aspects, the first amino acid is not His. In some aspects, the first amino acid is not Ile. In some aspects, the first amino acid is not Leu. In some aspects, the first amino acid is not Lys. In some aspects, the first amino acid is not Met. In some aspects, the first amino acid is not Phe. In some aspects. In some aspects, the first amino acid is not the first amino acid is not Pro. In some aspects. In some aspects, the first amino acid is not the first amino acid is not Ser. In some aspects, the first amino acid is not Thr. In some aspects, the first amino acid is not Trp. In some aspects, the first amino acid is not Tyr. In some aspects, the first amino acid is not Val.

The second amino acid can be any amino acid, including any of Ala, Arg, Asn, Asp, Cys, Glu, Gln, Gly, His, Ile, Leu, Lys, Met, Phe, Pro, Ser, Thr, Trp, Tyr, or Val. In some aspects, the second amino acid is Ala. In some aspects, the second amino acid is Arg. In some aspects, the second amino acid is Asn. In some aspects, the second amino acid is Asp. In some aspects, the second amino acid is Cys. In some aspects, the second amino acid is Glu. In some aspects, the second amino acid is Gln. In some aspects, the second amino acid is Gly. In some aspects, the second amino acid is His. In some aspects, the second amino acid is Ile. In some aspects, the second amino acid is Leu. In some aspects, the second amino acid is Lys. In some aspects, the second amino acid is Met. In some aspects, the second amino acid is Phe. In some aspects. In some aspects, the second amino acid is the second amino acid is Pro. In some aspects. In some aspects, the second amino acid is the second amino acid is Ser. In some aspects, the second amino acid is Thr. In some aspects, the second amino acid is Trp. In some aspects, the second amino acid is Tyr. In some aspects, the second amino acid is Val.

In some aspects, the second amino acid is not Ala. In some aspects, the second amino acid is not Arg. In some aspects, the second amino acid is not Asn. In some aspects, the second amino acid is not Asp. In some aspects, the second amino acid is not Cys. In some aspects, the second amino acid is not Glu. In some aspects, the second amino acid is not Gln. In some aspects, the second amino acid is not Gly. In some aspects, the second amino acid is not His. In some aspects, the second amino acid is not Ile. In some aspects, the second amino acid is not Leu. In some aspects, the second amino acid is not Lys. In some aspects, the second amino acid is not Met. In some aspects, the second amino acid is not Phe. In some aspects. In some aspects, the second amino acid is not the second amino acid is not Pro. In some aspects. In some aspects, the second amino acid is not the second amino acid is not Ser. In some aspects, the second amino acid is not Thr. In some aspects, the second amino acid is not Trp. In some aspects, the second amino acid is not Tyr. In some aspects, the second amino acid is not Val.

In some aspects, the second amino acid is glutamine, histidine, tryptophan, or cysteine.

The anticodon loop can comprise any anticodon sequence. In some aspects the anticodon loop sequence is UUU. In some aspects the anticodon loop sequence is UUC. In some aspects the anticodon loop sequence is UUA. In some aspects the anticodon loop sequence is UUG. In some aspects the anticodon loop sequence is CUU. In some aspects the anticodon loop sequence is CUC. In some aspects the anticodon loop sequence is CUA. In some aspects the anticodon loop sequence is CUG. In some aspects the anticodon loop sequence is AUU. In some aspects the anticodon loop sequence is AUC. In some aspects the anticodon loop sequence is AUA. In some aspects the anticodon loop sequence is AUG. In some aspects the anticodon loop sequence is GUU. In some aspects the anticodon loop sequence is GUC. In some aspects the anticodon loop sequence is GUA. In some aspects the anticodon loop sequence is GUG. In some aspects the anticodon loop sequence is UCU. In some aspects the anticodon loop sequence is UCC. In some aspects the anticodon loop sequence is UCA. In some aspects the anticodon loop sequence is UCG. In some aspects the anticodon loop sequence is CCU. In some aspects the anticodon loop sequence is CCC. In some aspects the anticodon loop sequence is CCA. In some aspects the anticodon loop sequence is CCG. In some aspects the anticodon loop sequence is ACU. In some aspects the anticodon loop sequence is ACC. In some aspects the anticodon loop sequence is ACA. In some aspects the anticodon loop sequence is ACG. In some aspects the anticodon loop sequence is GCU. In some aspects the anticodon loop sequence is GCC. In some aspects the anticodon loop sequence is GCA. In some aspects the anticodon loop sequence is GCG. In some aspects the anticodon loop sequence is UAU. In some aspects the anticodon loop sequence is UAC. In some aspects the anticodon loop sequence is UAA. In some aspects the anticodon loop sequence is UAG. In some aspects the anticodon loop sequence is CAU. In some aspects the anticodon loop sequence is CAC. In some aspects the anticodon loop sequence is CAA. In some aspects the anticodon loop sequence is CAG. In some aspects the anticodon loop sequence is AAU. In some aspects the anticodon loop sequence is AAC. In some aspects the anticodon loop sequence is AAA. In some aspects the anticodon loop sequence is AAG. In some aspects the anticodon loop sequence is GAU. In some aspects the anticodon loop sequence is GAC. In some aspects the anticodon loop sequence is GAA. In some aspects the anticodon loop sequence is GAG. In some aspects the anticodon loop sequence is UGU. In some aspects the anticodon loop sequence is UGC. In some aspects the anticodon loop sequence is UGA. In some aspects the anticodon loop sequence is UGG. In some aspects the anticodon loop sequence is CGU. In some aspects the anticodon loop sequence is CGC. In some aspects the anticodon loop sequence is CGA. In some aspects the anticodon loop sequence is CGG. In some aspects the anticodon loop sequence is AGU. In some aspects the anticodon loop sequence is AGC. In some aspects the anticodon loop sequence is AGA. In some aspects the anticodon loop sequence is AGG. In some aspects the anticodon loop sequence is GGU. In some aspects the anticodon loop sequence is GGC. In some aspects the anticodon loop sequence is GGA. In some aspects the anticodon loop sequence is GGG.

In some aspects the anticodon loop sequence is not UUU. In some aspects the anticodon loop sequence is not UUC. In some aspects the anticodon loop sequence is not UUA. In some aspects the anticodon loop sequence is not UUG. In some aspects the anticodon loop sequence is not CUU. In some aspects the anticodon loop sequence is not CUC. In some aspects the anticodon loop sequence is not CUA. In some aspects the anticodon loop sequence is not CUG. In some aspects the anticodon loop sequence is not AUU. In some aspects the anticodon loop sequence is not AUC. In some aspects the anticodon loop sequence is not AUA. In some aspects the anticodon loop sequence is not AUG. In some aspects the anticodon loop sequence is not GUU. In some aspects the anticodon loop sequence is not GUC. In some aspects the anticodon loop sequence is not GUA. In some aspects the anticodon loop sequence is not GUG. In some aspects the anticodon loop sequence is not UCU. In some aspects the anticodon loop sequence is not UCC. In some aspects the anticodon loop sequence is not UCA. In some aspects the anticodon loop sequence is not UCG. In some aspects the anticodon loop sequence is not CCU. In some aspects the anticodon loop sequence is not CCC. In some aspects the anticodon loop sequence is not CCA. In some aspects the anticodon loop sequence is not CCG. In some aspects the anticodon loop sequence is not ACU. In some aspects the anticodon loop sequence is not ACC. In some aspects the anticodon loop sequence is not ACA. In some aspects the anticodon loop sequence is not ACG. In some aspects the anticodon loop sequence is not GCU. In some aspects the anticodon loop sequence is not GCC. In some aspects the anticodon loop sequence is not GCA. In some aspects the anticodon loop sequence is not GCG. In some aspects the anticodon loop sequence is not UAU. In some aspects the anticodon loop sequence is not UAC. In some aspects the anticodon loop sequence is not UAA. In some aspects the anticodon loop sequence is not UAG. In some aspects the anticodon loop sequence is not CAU. In some aspects the anticodon loop sequence is not CAC. In some aspects the anticodon loop sequence is not CAA. In some aspects the anticodon loop sequence is not CAG. In some aspects the anticodon loop sequence is not AAU. In some aspects the anticodon loop sequence is not AAC. In some aspects the anticodon loop sequence is not AAA. In some aspects the anticodon loop sequence is not AAG. In some aspects the anticodon loop sequence is not GAU. In some aspects the anticodon loop sequence is not GAC. In some aspects the anticodon loop sequence is not GAA. In some aspects the anticodon loop sequence is not GAG. In some aspects the anticodon loop sequence is not UGU. In some aspects the anticodon loop sequence is not UGC. In some aspects the anticodon loop sequence is not UGA. In some aspects the anticodon loop sequence is not UGG. In some aspects the anticodon loop sequence is not CGU. In some aspects the anticodon loop sequence is not CGC. In some aspects the anticodon loop sequence is not CGA. In some aspects the anticodon loop sequence is not CGG. In some aspects the anticodon loop sequence is not AGU. In some aspects the anticodon loop sequence is not AGC. In some aspects the anticodon loop sequence is not AGA. In some aspects the anticodon loop sequence is not AGG. In some aspects the anticodon loop sequence is not GGU. In some aspects the anticodon loop sequence is not GGC. In some aspects the anticodon loop sequence is not GGA. In some aspects the anticodon loop sequence is not GGG.

The tRNA can comprise an anticodon loop sequence and an amino acid not encoded for by the anticodon loop. In certain aspects, the amino acid not encoded for by the anticodon loop is Arg. In certain aspects, the anticodon loop sequence does not encode for Arg.

In certain aspects, the anticodon loop sequence is UGC, GCA, GUC, UUC, GAA, UCC, GUG, GAU, UUU, UAG, UAA, CAU, GUU, UGG, UUG, UCG, UGA, GCU, UGU, UAC, GUA, AGC, CGC, CUG, CUC, CCC, GCC, AAU, UAU, AAG, CAA, CAG, CUU, CGG, AGA, CGA, AGU, CGU, CCA, AAC, CAC, or AGG. In certain aspects, the anticodon loop sequence is GUG, CUG, UUG, AGG, CGG, UGG, AAG, CAG, UAG, GCA, CCA, GCU, GCC, CCC, UCC. CUU, UUU, CGU, UGU, UAU, or CAU. In certain aspects, the anticodon loop sequence is CUG, UUG, GUG, CCA, or GCA, from 5′ to 3′. In some aspects, the anticodon loop sequence is not ACG, CCG, CCU, UCG, GCG, or UCU, from 5′ to 3′. In some aspects, wherein the mRNA sequence is not CGU, CGC, CGA, CGG, AGA, or AGG, from 5′ to 3′. In some aspects, the mRNA sequence is GCU, GCC, GCA, GCG, AAU, AAC, GAU, GAC, UGU, UGC, CAA, CAG, GAA, GAG, GGU, GGC, GGA, GGG, CAU, CAC, AUU, AUC, AUA, CUU, CUC, CUA, CUG, UUA, UUG, AAA, AAG, AUG, UUU, UUC, CCU, CCC, CCA, CCG, UCU, UCC, UCA, UCG, AGU, AGC, ACU, ACC, ACA, ACG, UGG, UAU, UAC, GUU, GUC, GUA, or GUG, from 5′ to 3′.

In certain aspects, the anticodon loop sequence is UGC, GCA, GUC, UUC, GAA, UCC, GUG, GAU, UUU, UAG, UAA, CAU, GUU, UGG, UUG, UCG, UGA, GCU, UGU, UAC, GUA, AGC, CGC, ACG, CCG, CCU, UCU, CUG, CUC, CCC, GCC, AAU, UAU, AAG, CAA, CAG, CUU, CGG, AGU, CGU, CCA, AAC, CAC, or AGG. In certain aspects, the anticodon loop sequence is GAA, CAA, UAA, AGG, CGG, UGG, AGU, CGU, UGU, AGC, CGC, UGC, GUA, GCA, ACG, GUU, AAU, GCC, CCU, or UCU, from 5′ to 3′. In certain aspects, the anticodon loop sequence is not AGA, CGA, GCU, UGA, ACU, or GGA, from 5′ to 3′. In some aspects, the mRNA sequence is not UCU, UCC, UCA, UCG, AGU, or AGC, from 5′ to 3′. In some aspects, the mRNA sequence is GCU, GCC, GCA, GCG, CGU, CGC, CGA, CGG, AGA, AGG, AAU, AAC, GAU, GAC, UGU, UGC, CAA, CAG, GAA, GAG, GGU, GGC, GGA, GGG, CAU, CAC, AUU, AUC, AUA, CUU, CUC, CUA, CUG, UUA, UUG, AAA, AAG, AUG, UUU, UUC, CCU, CCC, CCA, CCG, ACU, ACC, ACA, ACG, UGG, UAU, UAC, GUU, GUC, GUA, or GUG, from 5′ to 3′. In some aspects, the mRNA sequence is not UAA, UGA, or UAG.

The tRNA molecule can comprises a sequence having substitutions, deletion, or additions relative to a mammalian tRNA molecule. Disclosed are tRNA molecules comprising a sequence having at most one, two, three, four, or five substitutions relative to a mammalian tRNA molecule. In certain aspects, the mammalian tRNA molecule is a human tRNA molecule.

Also disclosed are nucleic acids encoding the tRNA sequences. The nucleic acid can comprise an expression vector. The nucleic acid can comprise a plasmid. The nucleic acids can be used to synthesize the tRNA sequences. Such synthesis can occur in a cell. The cell can be a bacterial cell, an insect cell, a yeast cell, a vertebrate cell, or any other cell capable of expressing the tRNA. The synthesis can occur by in vitro transcription.

Disclosed are nucleic acids having a sequence of any one of SEQ ID NOs: 1-144.

Also disclosed are tRNAs, or nucleic acids encoding for tRNAs, comprising one or more modifications to the tRNAs disclosed herein. The modifications may comprise 1, 2, 3, 4, 5, 6, 7, 8, 9, 10 or more modifications to the tRNA. Also disclosed are tRNAs, or nucleic acids encoding for tRNAs, comprising one or more modifications to the tRNAs of any one of SEQ ID NOs. 1-134. The modifications may comprise 1, 2, 3, 4, 5, 6, 7, 8, 9, 10 or more modifications to the tRNA. The modifications may comprise 1, 2, 3, 4, 5, 6, 7, 8, 9, 10 or more modifications to the tRNA of any one of SEQ ID NOs. 1-134.

Also disclosed are tRNAs having at least 85%, 86%, 87%, 88%, 89%, 90%, 91%, 92%, 93%, 94%, 95%, 96%, 97%, 98%, 99%, or any range derivable therein, sequence identity to any of the tRNAs disclosed herein. Also disclosed are nucleic acids encoding for tRNAs having at least 85%, 86%, 87%, 88%, 89%, 90%, 91%, 92%, 93%, 94%, 95%, 96%, 97%, 98%, 99%, or any range derivable therein, sequence identity to any of the tRNAs disclosed herein. Also disclosed are tRNAs having at least 85%, 86%, 87%, 88%, 89%, 90%, 91%, 92%, 93%, 94%, 95%, 96%, 97%, 98%, 99%, or any range derivable therein, sequence identity to any tRNA of SEQ ID NOs. 1-134. Also disclosed are nucleic acids encoding for tRNAs having at least 85%, 86%, 87%, 88%, 89%, 90%, 91%, 92%, 93%, 94%, 95%, 96%, 97%, 98%, 99%, or any range derivable therein, sequence identity to any tRNA of SEQ ID NOs. 1-134.

Also disclosed are vectors comprising any of the nucleic acids herein. The vector can be any vector, including any vector capable of delivering the nucleic acid to a cell of interest. In some aspects, the vector is a virus. In some aspects, the vector is an adenovirus, retrovirus, lentivirus, or adeno-associated virus (AAV), including any derivatives thereof.

Also disclosed are cells comprising any of the tRNA molecules disclosed herein. Also disclosed are cells comprising any of the nucleic acid disclosed herein. Also disclosed are cells comprising any of the vectors disclosed herein. The cells can be cells used for a cellular therapy, such as autologous stem cell therapies.

Disclosed herein are methods for making the tRNAs disclosed herein. In certain aspects, the method comprises contacting a nucleic acid comprising an anticodon loop with an amino acid that does is not naturally encoded for by the anticodon loop. In certain aspects, the nucleic acid is modified from a natural tRNA sequence to allow for an aminoacyl-tRNA synthetase to attach an amino acid, which is not normally encoded for by the tRNA, to the tRNA.

Disclosed herein are methods for modifying a protein produced by a gene using one or more of the tRNAs disclosed herein. Also disclosed are methods for producing a wild type protein from a gene having a missense mutation. Also disclosed are methods for correcting a missense mutation during translation of an mRNA. Also disclosed are methods for producing a genetically-engineered protein. Also disclosed are methods for introducing point mutations in a protein from an mRNA. The method can comprise 1, 2, 3, 4, 5, or more steps, including any of the following: translating an mRNA in the presence of one or more of the tRNAs disclosed herein, administering to a cell an effective amount of one or more of the tRNAs disclosed herein, administering to a cell an effective amount of one or more of the nucleic acids disclosed herein, administering to a cell an effective amount of one or more of the vectors disclosed herein, and detecting a protein from a cell. The administering can comprise any means of introducing the tRNA, nucleic acid, and/or vector to the cell, including by transfection, electroporation, or transduction. The cell can be any cell, such as a mammalian cell. The cell can be a human cell. The cell can be a cell comprising a missense mutation.

Disclosed herein are methods for treating or preventing a disease, such as a genetic disease, in a subject. Also disclosed are methods for treating or preventing Limb Girdle disease. Also disclosed are methods of restoring CAPN3 function in a cell. Also disclosed are methods of reversing the effects of CAPN3 loss-of-function in a cell. Also disclosed are methods of restoring CAPN3 function in a subject. Also disclosed are methods of reversing the effects of CAPN3 loss-of-function in a subject. Any of the methods can comprise 1, 2, 3, or more steps, including any of the following: administering a therapeutically effective amount of one or more of the tRNAs disclosed herein to the subject, administering a therapeutically effective amount of one or more of the nucleic acids disclosed herein to the subject, and administering a therapeutically effective amount of one or more of the vectors disclosed herein to the subject. The subject may have a genetic disease. The subject may have cancer. The disease, including the genetic disease or cancer, may be characterized, caused by, or accelerated by a single nucleotide variation (SNV). The SNV may result in a missense mutation in a gene. Genes disclosed herein that may have a missense mutation include ABCD1, CAPN3, GLA, GBA, GALC, ARSA, SGSH, HGSNAT, IDS, OTC, DHCR7, or HEXA. Disclosed herein are methods of correcting missense mutations in a gene. The gene may be ABCD1, CAPN3, GLA, GBA, GALC, ARSA, SGSH, HGSNAT, IDS, OTC, DHCR7, or HEXA. The gene may be any gene of Table 2. The SNV in the gene may be any SNV disclosed in Table 2. In some aspects, the SNV is recessive. In some aspects, the genetic disease is a recessive disease. The disease may be Adrenoleukodystrophy, Fabry disease, Gaucher disease type I, Metachromatic leukodystrophy, Mucopolysaccharidosis, Ornithine transcarbamylase deficiency, Smith-Lemli-Opitz syndrome, Tay-Sachs disease, Niemann-Pick disease, or Very long chain acyl-CoA dehydrogenase deficiency.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of Invention, Detailed Description, Claims, and description of Figure Legends.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Distribution of pathogenic missense mutations in human disease. A) Pie chart illustrating the distribution of known SNVs in ClinVar database among all SNVs (left) and among pathogenic SNVs (right). B) Few missense SNVs are common in the population and are reported to ClinVar many times, while most SNVs are low frequency and reported a small number of times. C) Among pathogenic missense mutations, not all amino acids are evenly give rise to pathogenic SNVs. D) At the codon level, Arg codons give rise to the plurality of pathogenic SNVs. Labeled are codon-level mutations where the maximum number of submitters for any individual SNVs is greater than or equal to 10.

FIGS. 2A-2F. Arginine isodecoder tRNAs can be engineered in read mutant codons and restore function to reporter proteins. A) Illustration of a tandem fluorescent reporter for Arginine mistranslation. In GFP, Arg96 is necessary for rapid fluorophore maturation; mutation of Arg96 results in a non-fluorescent GFP and a native RFP loading control. Expression of mutator MC-tRNA to decode mutant position 96 and deliver Arg restores GFP fluorescence. B) The construct in A was repeated for the full suite of potential SNVs than can turn an Arg codon into Cys, Trp, Gin, or His. C) (Top)(Left) Flow cytometry shows that GFP and RFP fluorescence scale together in positive control cells. (Right) the ratio of GFP to RFP is near 1 since the reporter has tandem proteins. (Bottom) for GFP* constructs where Arg96 is mutated as in A and B, GFP fluorescence does not increase with RFP. (Left) representative data from Arg96His construct. (Right) Traces of the GFP/RFP ratio for all mutant constructs confirming loss of fluorescence. D) Flow cytometry showing restoration of GFP fluorescence relative to RFP fluorescence loading control. When tRNA appropriate tRNA is expressed, GFP fluorescence is restored. E) Annexin stain (right) indicates early cell death; cell death increases with tRNA expression level. F) DAPI stain indicates late stage cell death. Cells with low level MC-tRNA expression do not experience toxicity.

FIGS. 3A-3C. Mass spectrometry indicates restoration of wild type sequence for disease associated peptides. A) Diagram of peptide expression construct. Since Arg>His_G>A SNV is associated with many SNVs from many diseases and many peptides, the inventors express a construct with tandem disease-associated peptides, separated by a Lys-C protease sites and a C-terminal affinity purification tag. A similar construct was made for each Arg-related SNV B) Representative LC-MS-MS data for peptide XYZ associated with Xyz syndrome. C) Summary peptide restoration among many disease-associated petites with expression of MC-tRNAArg.

FIGS. 4A-4F.: MC-tRNAs can be applied beyond arginine isoacceptors. A) Diagram of a fluorescent reporter to monitor Serine delivery at GFP position 65. Position 65 is mutated to ArgCCG, which disrupts GFP chromophore formation, resulting in a non-fluorescent cell. Delivery of serine to position 65 by MC-tRNAs restores fluorescence. B) Control transfections were done with plasmids expressing no tRNA, and either GFP only, RFP only, or a tandem GFP-RFP. Cells were filtered for RFP expression above the black line for further analysis. (Left) Fluorescence for GFP and RFP channels is shown for 24-hour post-transfection cells, a single replicate is shown. (Right) Histogram of GFP to RFP ratio is RFP and GFP-RFP expression cells. C) Plasmids with the tandem GFP*-RFP construct where Ser65 is mutated to Arg(CCG) from A and a tRNA construct were transfected into HEK293T cells. Fluorescence was monitored with flow cytometry at 24 hours, 48, and 72 hours post-transfection. GFP and RFP fluorescence for a single replicate at 24 hours is shown. D) Histogram of GFP to RFP ratio is shown for constructs in C. Expression of tRNASer(CGG), with several different backbones, all restore GFP fluorescence. Expression of tRNASer(CGA) does not restore fluorescence because it does not decode ArgCCG. E) Ratio of GFP to RFP is used as a proxy for tRNA expression and compared to AnnexinV stain for cell death. Increased GFP-RRFP ratio does not correlate with increased cell death. F) Annexin fluorescence was used to call cells as alive or dead using the threshold indicated by the dashed line in E. The fraction of dead cells was calculated for 3 replicates for each expression plasmid at 24 and 48 h.

FIGS. 5A-5C. Stable lines are viable and recover GFP. A) cells expressing WT GFP-RFP construct are poly-clonal. 2 population are visible, one with high levels of GFP fluorescence. Only cells staining low on DAPI live-dead stain are displayed. B) Cell expressing GFP-RFP construct where GFP bears the mutation R96C to abolish fluorescence. This population is low on GFP fluorescence. Only cells staining low on DAPI are displayed. C) Cells expressing Arg>Cys-repairing mc-tRNA are viable, though grow slowly. Displayed here are cells staining low on DAPI live-dead stain, and GFP fluorescence indicating low level repair of the GFP mutation R96C, compared to B.

FIGS. 6A-6C. Stable lines are viable and recover GFP. A) cells expressing WT GFP-RFP construct are poly-clonal. 2 population are visible, one with high levels of GFP fluorescence. Only cells staining low on DAPI live-dead stain are displayed. B) Cell expressing GFP-RFP construct where GFP bears the mutation R96Q to abolish fluorescence. This population is low on GFP fluorescence. Only cells staining low on DAPI are displayed. C) Cells expressing Arg>Gln-repairing mc-tRNA are viable, though grow slowly. Displayed here are cells staining low on DAPI live-dead stain, and GFP fluorescence indicating low level repair of the GFP mutation R96Q, compared to B.

FIGS. 7A-7E. me-tRNA expressing cells show minimal disruption to native gene expression. mRNA-sequencing was done on stable cell lines and compared with positive, negative, and null controls. In all figures, the Y axis is transcripts per million (TPM). A) Stable cell lines show negligible induction of the heat shock response compared to wild type cells. A positive control of heat shocked cells are included for comparison. B) Genes associated with apoptosis were measured in stable cell lines compared to untransfected cells and control stressed cells. Minimal induction of apoptosis pathways is observed. C) Genes associated with a general stress response further indicate minimal changes in gene expression, thus minimal disruption to cellular physiology. D) Gene's associated with an innate immune response were analyzed to see if there is an immunogenic effect to expression of mc-tRNAs in this context. Minimal changes to expression of these genes is observed. E) Expression of glycolytic enzyme GAPDH was used as a control since no changes in gene expression to this pathway are expected. These data confirm that global measurements of gene expression behave as expected.

FIGS. 8A-8E. Validation of GFP-based mistranslation reporters. (A) Flow cytometry analysis of GFP expression in cells overexpressing WT and mutant GFP-mCherry fusion proteins. All live single cells are displayed. Gates were set based on single color controls. (B) Density curves for GFP expression in cells overexpressing WT and mutant GFP-mCherry fusion proteins. (C)-(E) GFP signal normalized to mCherry for cells overexpressing GFP-mCherry fusion proteins with point mutations at GFP65 (C), GFP96 (D) and other GFP point mutations (E). Each dot represents one biological replicate. The mean and standard deviation for each sample are shown as a solid black dot and a vertical line respectively.

FIGS. 9A-9C. Validation of other fluorescence protein (FP)-based mistranslation reporters. (A) Flow cytometry analysis of mPlum expression in cells overexpressing WT and R96C eGFP-mPlum fusion proteins. All live single cells are displayed. Gates were set based on single color controls. (B) Density curves for mPlum expression in cells overexpressing WT and R96C eGFP-mPlum fusion proteins. (C) FP signal normalized to eGFP for cells overexpressing respective FP-based mistranslation reporters. Each dot represents one biological replicate. The mean and standard deviation for each sample are shown as a solid black dot and a vertical line respectively.

FIG. 10. Summary of all mistranslation reporters quantified. Each well is labelled with a fluorescence protein (FP) and the position of the mutation that acts as a mistranslation reporter. When WT amino acid (aa) is mutated to Mut aa at this position, the FP reporter signal decreases more than 2-fold. FP reporters that have been reported previously are colored in gray.

FIGS. 11A-11G. FIG. 3: Result of mctRNA Ser-tRNA^{Arg(CCG)}. (A) The sequence of Ser-tRNA^{Arg(CCG)} expression cassette. The tRNA sequence is highlighted in bold and the anticodon is underscored. 200 bp of endogenous sequences upstream as well as downstream of tRNA-Ser-CGA-1-1 gene was maintained to allow proper transcription and processing of the mctRNA. (B) Density curves for GFP signals normalized to mCherry in cells overexpressing i) WT GFP, ii) GFP(S65R), and iii) GFP(S65R) together with mctRNA (n=3). (C) Expression levels of Ser-tRNA^{Arg(CCG)} normalized to endogenous tRNA-Ser-CGA-1-1 in cells overexpressing GFP(S65R) with or without Ser-tRNA^{Arg(CCG)} (n=3). (D) Charging levels of endogenous tRNA-Ser-CGA-1-1 and Ser-tRNA^{Arg(CCG)} in cells overexpressing GFP(S65R) together with Ser-tRNA^{Arg(CCG)} (n=3). (E) Average mutation rates at each nucleotide position of endogenous tRNA-Ser-CGA-1-1 and Ser-tRNA^{Arg(CCG)} (n=3). (F) Differential gene expression in cells expressing GFP(S65R)-mCherry with mctRNA Ser-tRNA^{Arg(CCG)} versus without. (G) Biological process gene ontology enrichment analysis for significantly up- or down-regulated genes (p>0.05, absolute fold change (FC)>2). The solid vertical line marks p=0.05.

FIGS. 12A-12H. Result of mctRNA Arg-tRNA^{Cys/His/Gln/Trp(***)}. (A) GFP signals relative to WT GFP in cells overexpressing GFP(R96Q) together with mctRNA Arg-tRNA^{Gln(CUG)} of 19 different endogenous Arg tRNA isodecoder backbones. WT GFP and GFP(R96Q) are displayed for comparison. (B) The sequence of Arg-tRNA^{Cys/His/Gln/Trp(***)} expression cassette. The tRNA sequence is highlighted in bold and the anticodon region is underscored. The anticodon region was mutated to Cys, His, Gln and Trp anticodons to create cognate mctRNAs. 200 bp of endogenous sequences upstream as well as downstream of tRNA-Arg-CCT-4-1 was maintained to allow proper transcription and processing of the mctRNA. (C) Density curves of GFP signal normalized to mCherry for cells overexpressing i) WT GFP, ii) GFP R96 mutants, and iii) GFP R96 mutants together with corresponding mctRNAs. (D) IP-MS quantifications of mctRNA-corrected peptide SAMPEGYVQER in cells overexpressing GFP R96 mutants with and without corresponding mctRNAs (n=2). (E) MS spectra of mctRNA-corrected peptide SAMPEGYVQER in cells overexpressing GFP R96 mutants with corresponding mctRNAs. (F) Expression levels of Arg mctRNAs normalized to endogenous tRNA-Arg-CCT-4-1 in cells overexpressing GFP R96 mutants with or without corresponding mctRNA (n=3). (G) Charging levels of endogenous tRNA-Arg-CCT-4-1 and Arg mctRNAs in cells overexpressing GFP R96 mutants together with corresponding mctRNAs (n=3). (H) Average mutation rates at each nucleotide position of endogenous tRNA-Ser-CGA-1-1 and Arg mctRNAs (n=3).

FIGS. 13A-13E. Cellular response to Arg mctRNAs. (A) Differential gene expression in cells expressing GFP mutant-mCherry and the corresponding mctRNA versus cells only expressing GFP mutant-mCherry. (B)-(E) Biological process gene ontology (GO) enrichment analyses for significantly up- or down-regulated genes (p>0.05, absolute fold change (FC)>2) with Arg mctRNA expressions. The solid vertical line marks p=0.05. b-c. Top 5 GO terms with the highest fold enrichment are displayed for up- and down-regulated genes are displayed.

FIGS. 14A-14C. Arg-tRNA^{Gln(CUG)} rescued LGMD2A relevant protein CAPN3 mutant. (A) Schematic of Calpain3 protein domains and mutation sites. (B) Western blot for cells transfected with mock construct, WT CAPN3, catalytically dead mutant C129S, and deficient mutant R490Q with or without Arg-tRNA^{Gln(CUG)}. Full length (FL) CAPN3 (94 KDa), two bands of autolytic products (\textcircled{1} ~55 KDa, \textcircled{2} ~65 KDa), as well as GAPDH (37 KDa), are marked in the figure. GAPDH is the loading control. (C) Quantification for relative western blot band intensities (biological replicates n=4).

### DETAILED DESCRIPTION OF THE INVENTION

Many genetic diseases are caused by a mutation in protein-coding regions of DNA that result in changing the amino acid identity in the protein product (missense). The resulting mutant protein may be biologically inactive, thus incurring crucial loss of function leading to disease. Disclosed are specific missense correcting tRNAs (MC-tRNAs) for protein biosynthesis in cells. MC-tRNA has a covalently attached (aminoacylated or charged) amino acid that does not match the anticodon sequence for reading codons of the charged amino acid. These engineered MC-tRNs can restore the original protein sequence during translation, thereby producing functional proteins and alter disease outcomes. Disclosed are MC-tRNA capable of correcting specific Arg or Ser mutations to functional proteins by the respective MC-tRNA^{Arg} and MC-tRNA^{Ser}. Also disclosed is the restoration of the wild-type sequence of disease-relevant peptides by MC-tRNA^{Arg}. Also disclosed are MC-tRNAs that natively deliver arginine to treat genetic diseases that are derived from missense mutation of Arg residues. Certain aspects correct single base mutations that lead to disease, including mutations of Arg to a different amino acid, which is the most prevalent mutation leading to disease.

Disclosed are specific missense correcting tRNAs (MC-tRNA^{xxx}(yyy) where xxx=amino acid attached to the 3′ end, yyy=anticodon sequence) for protein biosynthesis in cells. MC-tRNA has a covalently attached (charged) amino acid that does not match the anticodon sequence for reading codons of the charged amino acid. Use of engineered MC-tRNAs can correct genetic diseases derived from missense mutations such as Adrenoleukodystrophy, Sanfilippo (MPS-III-A), Very long chain acyl-CoA dehydrogenase deficiency (VLCADD), among many others. This approach, in certain aspects, is well suited for recessive diseases where restoration of a small amount of native protein activity can correct the phenotype. Certain aspects focus on orphan metabolic diseases with infant onset and poor prognosis.

Certain aspects concern the identification of an uneven distribution of specific types of mutations, and mutations from Arg to Cys, Trp, Gln, and His are the most prevalent, accounting for 9% of all pathogenic SNVs. Disclosed are aspects concerning correcting genetic disease by Co-translational missense correction of genetic diseases (CoMED).

### I. Obtaining Nucleotides

### A. Synthesis

The nucleic acid molecules, including tRNAs or nucleic acids encoding the tRNAs described herein, may be generated by nucleic acid synthesis. The tRNAs or nucleic acids encoding the tRNAs may be synthesized using any method known in the art, such as phosphoramidite synthesis and/or solid-phase synthesis. tRNAs or nucleic acids encoding the tRNAs may be synthesized.

### B. Expression

The nucleic acid molecules, including any tRNAs or nucleic acids encoding the tRNAs described herein, may be generated by expression vectors. The expression vectors used herein may contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, and a selectable marker element. Such sequences and methods of using the same are well known in the art.

### 1. Expression Systems

Numerous expression systems exist that comprise at least a part or all of the expression vectors discussed above. Prokaryote- and/or eukaryote-based systems can be employed for use with an aspect to produce nucleic acid sequences. Commercially and widely available systems include but are not limited to bacterial, mammalian, yeast, and insect cell systems. Those skilled in the art are able to express a vector to produce a nucleic acid sequence using an appropriate expression system.

### 2. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 3. Host Cells

In another aspect, contemplated are the use of host cells into which a recombinant expression vector has been introduced. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### II. Pharmaceutical Compositions

In certain aspects, the compositions or agents, including those for use in the methods disclosed herein, such as tRNAs, nucleic acids encoding the tRNAs, vectors comprising the tRNAs, or cells comprising the tRNAs, are suitably contained in a pharmaceutically acceptable carrier. The carrier can be non-toxic, biocompatible, and selected so as not to detrimentally affect the biological activity of the agent. The agents in some aspects of the disclosure may be formulated into preparations for local delivery (i.e. to a specific location of the body, such as the brain, nervous tissue, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen.

### A. Proteins

The nucleotides as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### B. Other Agents

It is contemplated that other agents may be used in combination with certain aspects. These additional agents include agents that act in combination and/or synergistically with the tRNAs, nucleic acids encoding the tRNAs, vectors comprising the tRNAs, or cells comprising the tRNAs described herein. The additional agents may comprise agents that reduce symptoms of the disorders disclosed herein, or may comprise agents that reduce side effects associated with the therapeutic compositions disclosed herein.

### III. Sequences and Single Nucleotide Variations

TABLE 1
Nucleic Acids for Aspects Disclosed Herein

| | SEQ | |
|---|---|---|
| Name | ID NO | Sequence |
| tRNA^{Arg} | 1 | GGGCCAGTGGCGCAATGGATAACGCGTCTGACTCTGG |
| CTG1 | | ATCAGAAGATTCCAGGTTCGACTCCTGGCTGGCTCGcca |
| tRNA^{Arg} | 2 | GGGCCAGTGGCGCAATGGATAACGCGTCTGACTCTGG |
| CTG2 | | ATCAGAAGATTCTAGGTTCGACTCCTGGCTGGCTCGcca |
| tRNA^{Arg} | 3 | GGCCGCGTGGCCTAATGGATAAGGCGTCTGATTCTGGA |
| CTG3 | | TCAGAAGATTGAGGGTTCGAGTCCCTTCGTGGTCGcca |
| tRNA^{Arg} | 4 | GACCCAGTGGCCTAATGGATAAGGCATCAGCCTCTGG |
| CTG4 | | AGCTGGGGATTGTGGGTTCGAGTCCCATCTGGGTCGcca |
| tRNA^{Arg} | 5 | GCCCCAGTGGCCTAATGGATAAGGCACTGGCCTCTGAA |
| CTG5 | | GCCAGGGATTGTGGGTTCGAGTCCCACCTGGGGTAcca |
| tRNA^{Arg} | 6 | GCCCCAGTGGCCTAATGGATAAGGCACTGGCCTCTGAA |
| CTG6 | | GCCAGGGATTGTGGGTTCGAGTCCCACCTGGGGTGcca |
| tRNA^{Arg} | 7 | GCCCCGGTGGCCTAATGGATAAGGCATTGGCCTCTGAA |
| CTG7 | | GCCAGGGATTGTGGGTTCGAGTCCCACCCGGGGTAcca |
| tRNA^{Arg} | 8 | GCCCCAGTGGCCTAATGGATAAGGCATTGGCCTCTGAA |
| CTG8 | | GCCAGGGATTGTGGGTTCGAGTCCCATCTGGGGTGcca |
| tRNA^{Arg} | 9 | GCCCCAGTGGCCTGATGGATAAGGTACTGGCCTCTGAA |
| CTG9 | | GCCAGGGATTGTGGGTTCGAGTTCCACCTGGGGTAcca |
| tRNA^{Arg} | 10 | GGCCGCGTGGCCTAATGGATAAGGCGTCTGACTCTGGA |
| CTG10 | | TCAGAAGATTGCAGGTTCGAGTCCTGCCGCGGTCGcca |
| tRNA^{Arg} | 11 | GACCACGTGGCCTAATGGATAAGGCGTCTGACTCTGGA |
| CTG11 | | TCAGAAGATTGAGGGTTCGAATCCCTCCGTGGTTAcca |
| tRNA^{Arg} | 12 | GACCGCGTGGCCTAATGGATAAGGCGTCTGACTCTGGA |
| CTG12 | | TCAGAAGATTGAGGGTTCGAGTCCCTTCGTGGTCGcca |
| tRNA^{Arg} | 13 | GACCACGTGGCCTAATGGATAAGGCGTCTGACTCTGGA |
| CTG13 | | TCAGAAGATTGAGGGTTCGAATCCCTTCGTGGTTAcca |
| tRNA^{Arg} | 14 | GACCACGTGGCCTAATGGATAAGGCGTCTGACTCTGGA |
| CTG14 | | TCAGAAGATTGAGGGTTCGAATCCCTTCGTGGTTGcca |
| tRNA^{Arg} | 15 | GGCCGTGTGGCCTAATGGATAAGGCGTCTGACTCTGGA |
| CTG15 | | TCAAAAGATTGCAGGTTTGAGTTCTGCCACGGTCGcca |
| tRNA^{Arg} | 16 | GGCTCCGTGGCGCAATGGATAGCGCATTGGACTCTGAA |
| CTG16 | | TTCAAAGGTTCCGGGTTCGAGTCCCGGCGGAGTCGcca |
| tRNA^{Arg} | 17 | GGCTCTGTGGCGCAATGGATAGCGCATTGGACTCTGAA |
| CTG17 | | TTCAAAGGTTGTGGGTTCGAATCCCACCAGAGTCGcca |
| tRNA^{Arg} | 18 | GGCTCTGTGGCGCAATGGATAGCGCATTGGACTCTGAA |
| CTG18 | | TTCAAAGGTTGTGGGTTCGAGTCCCACCAGAGTCGcca |
| tRNA^{Arg} | 19 | GTCTCTGTGGCGCAATGGACGAGCGCGCTGGACTCTGA |
| CTG19 | | ATCCAGAGGTTCCGGGTTCGAGTCCCGGCAGAGATGcca |
| tRNA^{Arg} | 20 | GGCTCTGTGGCGCAATGGATAGCGCATTGGACTCTGAA |
| CTG20 | | TTCAAAGGTTGCGGGTTCGAGTCCCTCCAGAGTCGcca |
| tRNA^{Arg} | 21 | GGCTCCGTGGCGCAATGGATAGCGCATTGGACTCTGAG |
| CTG21 | | AGGCTGAAGGCATTCAAAGGTTCCGGGTTCGAGTCCCG |
| | | GCGGAGTCGcca |
| tRNA^{Arg} | 22 | GGCTCTGTGGCGCAATGGATAGCGCATTGGACTCTGAG |
| CTG22 | | TGACGAATAGAGCAATTCAAAGGTTGTGGGTTCGAATC |
| | | CCACCAGAGTCGcca |
| tRNA^{Arg} | 23 | GGCTCTGTGGCGCAATGGATAGCGCATTGGACTCTGAG |
| CTG23 | | CTGAGCCTAGTGTGGTCATTCAAAGGTTGTGGGTTCGA |
| | | GTCCCACCAGAGTCGcca |
| tRNA^{Arg} | 24 | GGCTCTGTGGCGCAATGGATAGCGCATTGGACTCTGAG |
| CTG24 | | ATAGTTAGAGAAATTCAAAGGTTGTGGGTTCGAGTCCC |
| | | ACCAGAGTCGcca |
| tRNA^{Arg} | 25 | GTCTCTGTGGCGCAATGGACGAGCGCGCTGGACTCTGA |
| CTG25 | | ATCCAGAGGTTCCGGGTTCGAGTCCCGGCAGAGATGcca |
| tRNA^{Arg} | 26 | GGCTCTGTGGCGCAATGGATAGCGCATTGGACTCTGAG |
| CTG26 | | CCTAAATCAAGAGATTCAAAGGTTGCGGGTTCGAGTCC |
| | | CTCCAGAGTCGcca |
| tRNA^{Arg} | 27 | GGGCCAGTGGCGCAATGGATAACGCGTCTGACTTTGG |
| TTG1 | | ATCAGAAGATTCCAGGTTCGACTCCTGGCTGGCTCGcca |
| tRNA^{Arg} | 28 | GGGCCAGTGGCGCAATGGATAACGCGTCTGACTTTGG |
| TTG2 | | ATCAGAAGATTCTAGGTTCGACTCCTGGCTGGCTCGcca |
| tRNA^{Arg} | 29 | GGCCGCGTGGCCTAATGGATAAGGCGTCTGATTTTGGA |
| TTG3 | | TCAGAAGATTGAGGGTTCGAGTCCCTTCGTGGTCGcca |
| tRNA^{Arg} | 30 | GACCCAGTGGCCTAATGGATAAGGCATCAGCCTTTGGA |
| TTG4 | | GCTGGGGATTGTGGGTTCGAGTCCCATCTGGGTCGcca |
| tRNA^{Arg} | 31 | GCCCCAGTGGCCTAATGGATAAGGCACTGGCCTTTGAA |
| TTG5 | | GCCAGGGATTGTGGGTTCGAGTCCCACCTGGGGTAcca |
| tRNA^{Arg} | 32 | GCCCCAGTGGCCTAATGGATAAGGCACTGGCCTTTGAA |
| TTG6 | | GCCAGGGATTGTGGGTTCGAGTCCCACCTGGGGTGcca |
| tRNA^{Arg} | 33 | GCCCCGGTGGCCTAATGGATAAGGCATTGGCCTTTGAA |
| TTG7 | | GCCAGGGATTGTGGGTTCGAGTCCCACCCGGGGTAcca |
| tRNA^{Arg} | 34 | GCCCCAGTGGCCTAATGGATAAGGCATTGGCCTTTGAA |
| TTG8 | | GCCAGGGATTGTGGGTTCGAGTCCCATCTGGGGTGcca |
| tRNA^{Arg} | 35 | GCCCCAGTGGCCTGATGGATAAGGTACTGGCCTTTGAA |
| TTG9 | | GCCAGGGATTGTGGGTTCGAGTTCCACCTGGGGTAcca |
| tRNA^{Arg} | 36 | GGCCGCGTGGCCTAATGGATAAGGCGTCTGACTTTGGA |
| TTG10 | | TCAGAAGATTGCAGGTTCGAGTCCTGCCGCGGTCGcca |
| tRNA^{Arg} | 37 | GACCACGTGGCCTAATGGATAAGGCGTCTGACTTTGGA |
| TTG11 | | TCAGAAGATTGAGGGTTCGAATCCCTCCGTGGTTAcca |
| tRNA^{Arg} | 38 | GACCGCGTGGCCTAATGGATAAGGCGTCTGACTTTGGA |
| TTG12 | | TCAGAAGATTGAGGGTTCGAGTCCCTTCGTGGTCGcca |
| tRNA^{Arg} | 39 | GACCACGTGGCCTAATGGATAAGGCGTCTGACTTTGGA |
| TTG13 | | TCAGAAGATTGAGGGTTCGAATCCCTTCGTGGTTAcca |
| tRNA^{Arg} | 40 | GACCACGTGGCCTAATGGATAAGGCGTCTGACTTTGGA |
| TTG14 | | TCAGAAGATTGAGGGTTCGAATCCCTTCGTGGTTGcca |
| tRNA^{Arg} | 41 | GGCCGTGTGGCCTAATGGATAAGGCGTCTGACTTTGGA |
| TTG15 | | TCAAAAGATTGCAGGTTTGAGTTCTGCCACGGTCGcca |
| tRNA^{Arg} | 42 | GGCTCCGTGGCGCAATGGATAGCGCATTGGACTTTGAA |
| TTG16 | | TTCAAAGGTTCCGGGTTCGAGTCCCGGCGGAGTCGcca |
| tRNA^{Arg} | 43 | GGCTCTGTGGCGCAATGGATAGCGCATTGGACTTTGAA |
| TTG17 | | TTCAAAGGTTGTGGGTTCGAATCCCACCAGAGTCGcca |
| tRNA^{Arg} | 44 | GGCTCTGTGGCGCAATGGATAGCGCATTGGACTTTGAA |
| TTG18 | | TTCAAAGGTTGTGGGTTCGAGTCCCACCAGAGTCGcca |
| tRNA^{Arg} | 45 | GTCTCTGTGGCGCAATGGACGAGCGCGCTGGACTTTGA |
| TTG19 | | ATCCAGAGGTTCCGGGTTCGAGTCCCGGCAGAGATGcca |
| tRNA^{Arg} | 46 | GGCTCTGTGGCGCAATGGATAGCGCATTGGACTTTGAA |
| TTG20 | | TTCAAAGGTTGCGGGTTCGAGTCCCTCCAGAGTCGcca |
| tRNA^{Arg} | 47 | GGCTCCGTGGCGCAATGGATAGCGCATTGGACTTTGAG |
| TTG21 | | AGGCTGAAGGCATTCAAAGGTTCCGGGTTCGAGTCCCG |
| | | GCGGAGTCGcca |
| tRNA^{Arg} | 48 | GGCTCTGTGGCGCAATGGATAGCGCATTGGACTTTGAG |
| TTG22 | | TGACGAATAGAGCAATTCAAAGGTTGTGGGTTCGAATC |
| | | CCACCAGAGTCGcca |
| tRNA^{Arg} | 49 | GGCTCTGTGGCGCAATGGATAGCGCATTGGACTTTGAG |
| TTG23 | | CTGAGCCTAGTGTGGTCATTCAAAGGTTGTGGGTTCGA |
| | | GTCCCACCAGAGTCGcca |
| tRNA^{Arg} | 50 | GGCTCTGTGGCGCAATGGATAGCGCATTGGACTTTGAG |
| TTG24 | | ATAGTTAGAGAAATTCAAAGGTTGTGGGTTCGAGTCCC |
| | | ACCAGAGTCGcca |
| tRNA^{Arg} | 51 | GTCTCTGTGGCGCAATGGACGAGCGCGCTGGACTTTGA |
| TTG25 | | ATCCAGAGGTTCCGGGTTCGAGTCCCGGCAGAGATGcca |
| tRNA^{Arg} | 52 | GGCTCTGTGGCGCAATGGATAGCGCATTGGACTTTGAG |
| TTG26 | | CCTAAATCAAGAGATTCAAAGGTTGCGGGTTCGAGTCC |
| | | CTCCAGAGTCGcca |
| tRNA^{Arg} | 53 | GGGCCAGTGGCGCAATGGATAACGCGTCTGACTGTGG |
| GTG1 | | ATCAGAAGATTCCAGGTTCGACTCCTGGCTGGCTCGcca |
| tRNA^{Arg} | 54 | GGGCCAGTGGCGCAATGGATAACGCGTCTGACTGTGG |
| GTG2 | | ATCAGAAGATTCTAGGTTCGACTCCTGGCTGGCTCGcca |
| tRNA^{Arg} | 55 | GGCCGCGTGGCCTAATGGATAAGGCGTCTGATTGTGGA |
| GTG3 | | TCAGAAGATTGAGGGTTCGAGTCCCTTCGTGGTCGcca |
| tRNA^{Arg} | 56 | GACCCAGTGGCCTAATGGATAAGGCATCAGCCTGTGG |
| GTG4 | | AGCTGGGGATTGTGGGTTCGAGTCCCATCTGGGTCGcca |
| tRNA^{Arg} | 57 | GCCCCAGTGGCCTAATGGATAAGGCACTGGCCTGTGA |
| GTG5 | | AGCCAGGGATTGTGGGTTCGAGTCCCACCTGGGGTAcca |
| tRNA^{Arg} | 58 | GCCCCAGTGGCCTAATGGATAAGGCACTGGCCTGTGA |
| GTG6 | | AGCCAGGGATTGTGGGTTCGAGTCCCACCTGGGGTGcca |
| tRNA^{Arg} | 59 | GCCCCGGTGGCCTAATGGATAAGGCATTGGCCTGTGAA |
| GTG7 | | GCCAGGGATTGTGGGTTCGAGTCCCACCCGGGGTAcca |
| tRNA^{Arg} | 60 | GCCCCAGTGGCCTAATGGATAAGGCATTGGCCTGTGAA |
| GTG8 | | GCCAGGGATTGTGGGTTCGAGTCCCATCTGGGGTGcca |
| tRNA^{Arg} | 61 | GCCCCAGTGGCCTGATGGATAAGGTACTGGCCTGTGAA |
| GTG9 | | GCCAGGGATTGTGGGTTCGAGTTCCACCTGGGGTAcca |
| tRNA^{Arg} | 62 | GGCCGCGTGGCCTAATGGATAAGGCGTCTGACTGTGG |
| GTG10 | | ATCAGAAGATTGCAGGTTCGAGTCCTGCCGCGGTCGcca |
| tRNA^{Arg} | 63 | GACCACGTGGCCTAATGGATAAGGCGTCTGACTGTGG |
| GTG11 | | ATCAGAAGATTGAGGGTTCGAATCCCTCCGTGGTTAcca |
| tRNA^{Arg} | 64 | GACCGCGTGGCCTAATGGATAAGGCGTCTGACTGTGG |
| GTG12 | | ATCAGAAGATTGAGGGTTCGAGTCCCTTCGTGGTCGcca |
| tRNA^{Arg} | 65 | GACCACGTGGCCTAATGGATAAGGCGTCTGACTGTGG |
| GTG13 | | ATCAGAAGATTGAGGGTTCGAATCCCTTCGTGGTTAcca |
| tRNA^{Arg} | 66 | GACCACGTGGCCTAATGGATAAGGCGTCTGACTGTGG |
| GTG14 | | ATCAGAAGATTGAGGGTTCGAATCCCTTCGTGGTTGcca |
| tRNA^{Arg} | 67 | GGCCGTGTGGCCTAATGGATAAGGCGTCTGACTGTGGA |
| GTG15 | | TCAAAAGATTGCAGGTTTGAGTTCTGCCACGGTCGcca |
| tRNA^{Arg} | 68 | GGCTCCGTGGCGCAATGGATAGCGCATTGGACTGTGA |
| GTG16 | | ATTCAAAGGTTCCGGGTTCGAGTCCCGGCGGAGTCGcca |
| tRNA^{Arg} | 69 | GGCTCTGTGGCGCAATGGATAGCGCATTGGACTGTGAA |
| GTG17 | | TTCAAAGGTTGTGGGTTCGAATCCCACCAGAGTCGcca |
| tRNA^{Arg} | 70 | GGCTCTGTGGCGCAATGGATAGCGCATTGGACTGTGAA |
| GTG18 | | TTCAAAGGTTGTGGGTTCGAGTCCCACCAGAGTCGcca |
| tRNA^{Arg} | 71 | GTCTCTGTGGCGCAATGGACGAGCGCGCTGGACTGTGA |
| GTG19 | | ATCCAGAGGTTCCGGGTTCGAGTCCCGGCAGAGATGcca |
| tRNA^{Arg} | 72 | GGCTCTGTGGCGCAATGGATAGCGCATTGGACTGTGAA |
| GTG20 | | TTCAAAGGTTGCGGGTTCGAGTCCCTCCAGAGTCGcca |
| tRNA^{Arg} | 73 | GGCTCCGTGGCGCAATGGATAGCGCATTGGACTGTGA |
| GTG21 | | GAGGCTGAAGGCATTCAAAGGTTCCGGGTTCGAGTCCC |
| | | GGCGGAGTCGcca |
| tRNA^{Arg} | 74 | GGCTCTGTGGCGCAATGGATAGCGCATTGGACTGTGAG |
| GTG22 | | TGACGAATAGAGCAATTCAAAGGTTGTGGGTTCGAATC |
| | | CCACCAGAGTCGcca |
| tRNA^{Arg} | 75 | GGCTCTGTGGCGCAATGGATAGCGCATTGGACTGTGAG |
| GTG23 | | CTGAGCCTAGTGTGGTCATTCAAAGGTTGTGGGTTCGA |
| | | GTCCCACCAGAGTCGcca |
| tRNA^{Arg} | 76 | GGCTCTGTGGCGCAATGGATAGCGCATTGGACTGTGAG |
| GTG24 | | ATAGTTAGAGAAATTCAAAGGTTGTGGGTTCGAGTCCC |
| | | ACCAGAGTCGcca |
| tRNA^{Arg} | 77 | GTCTCTGTGGCGCAATGGACGAGCGCGCTGGACTGTGA |
| GTG25 | | ATCCAGAGGTTCCGGGTTCGAGTCCCGGCAGAGATGcca |
| tRNA^{Arg} | 78 | GGCTCTGTGGCGCAATGGATAGCGCATTGGACTGTGAG |
| GTG26 | | CCTAAATCAAGAGATTCAAAGGTTGCGGGTTCGAGTCC |
| | | CTCCAGAGTCGcca |
| tRNA^{Arg} | 79 | GGGCCAGTGGCGCAATGGATAACGCGTCTGACTCCAG |
| CCA1 | | ATCAGAAGATTCCAGGTTCGACTCCTGGCTGGCTCGcca |
| tRNA^{Arg} | 80 | GGGCCAGTGGCGCAATGGATAACGCGTCTGACTCCAG |
| CCA2 | | ATCAGAAGATTCTAGGTTCGACTCCTGGCTGGCTCGcca |
| tRNA^{Arg} | 81 | GGCCGCGTGGCCTAATGGATAAGGCGTCTGATTCCAGA |
| CCA3 | | TCAGAAGATTGAGGGTTCGAGTCCCTTCGTGGTCGcca |
| tRNA^{Arg} | 82 | GACCCAGTGGCCTAATGGATAAGGCATCAGCCTCCAG |
| CCA4 | | AGCTGGGGATTGTGGGTTCGAGTCCCATCTGGGTCGcca |
| tRNA^{Arg} | 83 | GCCCCAGTGGCCTAATGGATAAGGCACTGGCCTCCAA |
| CCA5 | | AGCCAGGGATTGTGGGTTCGAGTCCCACCTGGGGTAcca |
| tRNA^{Arg} | 84 | GCCCCAGTGGCCTAATGGATAAGGCACTGGCCTCCAA |
| CCA6 | | AGCCAGGGATTGTGGGTTCGAGTCCCACCTGGGGTGcca |
| tRNA^{Arg} | 85 | GCCCCGGTGGCCTAATGGATAAGGCATTGGCCTCCAAA |
| CCA7 | | GCCAGGGATTGTGGGTTCGAGTCCCACCCGGGGTAcca |
| tRNA^{Arg} | 86 | GCCCCAGTGGCCTAATGGATAAGGCATTGGCCTCCAAA |
| CCA8 | | GCCAGGGATTGTGGGTTCGAGTCCCATCTGGGGTGcca |
| tRNA^{Arg} | 87 | GCCCCAGTGGCCTGATGGATAAGGTACTGGCCTCCAAA |
| CCA9 | | GCCAGGGATTGTGGGTTCGAGTTCCACCTGGGGTAcca |
| tRNA^{Arg} | 88 | GGCCGCGTGGCCTAATGGATAAGGCGTCTGACTCCAG |
| CCA10 | | ATCAGAAGATTGCAGGTTCGAGTCCTGCCGCGGTCGcca |
| tRNA^{Arg} | 89 | GACCACGTGGCCTAATGGATAAGGCGTCTGACTCCAG |
| CCA11 | | ATCAGAAGATTGAGGGTTCGAATCCCTCCGTGGTTAcca |
| tRNA^{Arg} | 90 | GACCGCGTGGCCTAATGGATAAGGCGTCTGACTCCAG |
| CCA12 | | ATCAGAAGATTGAGGGTTCGAGTCCCTTCGTGGTCGcca |
| tRNA^{Arg} | 91 | GACCACGTGGCCTAATGGATAAGGCGTCTGACTCCAG |
| CCA13 | | ATCAGAAGATTGAGGGTTCGAATCCCTTCGTGGTTAcca |
| tRNA^{Arg} | 92 | GACCACGTGGCCTAATGGATAAGGCGTCTGACTCCAG |
| CCA14 | | ATCAGAAGATTGAGGGTTCGAATCCCTTCGTGGTTGcca |
| tRNA^{Arg} | 93 | GGCCGTGTGGCCTAATGGATAAGGCGTCTGACTCCAGA |
| CCA15 | | TCAAAAGATTGCAGGTTTGAGTTCTGCCACGGTCGcca |
| tRNA^{Arg} | 94 | GGCTCCGTGGCGCAATGGATAGCGCATTGGACTCCAA |
| CCA16 | | ATTCAAAGGTTCCGGGTTCGAGTCCCGGCGGAGTCGcca |
| tRNA^{Arg} | 95 | GGCTCTGTGGCGCAATGGATAGCGCATTGGACTCCAAA |
| CCA17 | | TTCAAAGGTTGTGGGTTCGAATCCCACCAGAGTCGcca |
| tRNA^{Arg} | 96 | GGCTCTGTGGCGCAATGGATAGCGCATTGGACTCCAAA |
| CCA18 | | TTCAAAGGTTGTGGGTTCGAGTCCCACCAGAGTCGcca |
| tRNA^{Arg} | 97 | GTCTCTGTGGCGCAATGGACGAGCGCGCTGGACTCCAA |
| CCA19 | | ATCCAGAGGTTCCGGGTTCGAGTCCCGGCAGAGATGcca |
| tRNA^{Arg} | 98 | GGCTCTGTGGCGCAATGGATAGCGCATTGGACTCCAAA |
| CCA20 | | TTCAAAGGTTGCGGGTTCGAGTCCCTCCAGAGTCGcca |
| tRNA^{Arg} | 99 | GGCTCCGTGGCGCAATGGATAGCGCATTGGACTCCAA |
| CCA21 | | GAGGCTGAAGGCATTCAAAGGTTCCGGGTTCGAGTCCC |
| | | GGCGGAGTCGcca |
| tRNA^{Arg} | 100 | GGCTCTGTGGCGCAATGGATAGCGCATTGGACTCCAAG |
| CCA22 | | TGACGAATAGAGCAATTCAAAGGTTGTGGGTTCGAATC |
| | | CCACCAGAGTCGcca |
| tRNA^{Arg} | 101 | GGCTCTGTGGCGCAATGGATAGCGCATTGGACTCCAAG |
| CCA23 | | CTGAGCCTAGTGTGGTCATTCAAAGGTTGTGGGTTCGA |
| | | GTCCCACCAGAGTCGcca |
| tRNA^{Arg} | 102 | GGCTCTGTGGCGCAATGGATAGCGCATTGGACTCCAAG |
| CCA24 | | ATAGTTAGAGAAATTCAAAGGTTGTGGGTTCGAGTCCC |
| | | ACCAGAGTCGcca |
| tRNA^{Arg} | 103 | GTCTCTGTGGCGCAATGGACGAGCGCGCTGGACTCCAA |
| CCA25 | | ATCCAGAGGTTCCGGGTTCGAGTCCCGGCAGAGATGcca |
| tRNA^{Arg} | 104 | GGCTCTGTGGCGCAATGGATAGCGCATTGGACTCCAAG |
| CCA26 | | CCTAAATCAAGAGATTCAAAGGTTGCGGGTTCGAGTCC |
| | | CTCCAGAGTCGcca |
| tRNA^{Arg} | 105 | GGGCCAGTGGCGCAATGGATAACGCGTCTGACTGCAG |
| GCA1 | | ATCAGAAGATTCCAGGTTCGACTCCTGGCTGGCTCGcca |
| tRNA^{Arg} | 106 | GGGCCAGTGGCGCAATGGATAACGCGTCTGACTGCAG |
| GCA2 | | ATCAGAAGATTCTAGGTTCGACTCCTGGCTGGCTCGcca |
| tRNA^{Arg} | 107 | GGCCGCGTGGCCTAATGGATAAGGCGTCTGATTGCAG |
| GCA3 | | ATCAGAAGATTGAGGGTTCGAGTCCCTTCGTGGTCGcca |
| tRNA^{Arg} | 108 | GACCCAGTGGCCTAATGGATAAGGCATCAGCCTGCAG |
| GCA4 | | AGCTGGGGATTGTGGGTTCGAGTCCCATCTGGGTCGcca |
| tRNA^{Arg} | 109 | GCCCCAGTGGCCTAATGGATAAGGCACTGGCCTGCAA |
| GCA5 | | AGCCAGGGATTGTGGGTTCGAGTCCCACCTGGGGTAcca |
| tRNA^{Arg} | 110 | GCCCCAGTGGCCTAATGGATAAGGCACTGGCCTGCAA |
| GCA6 | | AGCCAGGGATTGTGGGTTCGAGTCCCACCTGGGGTGcca |
| tRNA^{Arg} | 111 | GCCCCGGTGGCCTAATGGATAAGGCATTGGCCTGCAA |
| GCA7 | | AGCCAGGGATTGTGGGTTCGAGTCCCACCCGGGGTAcca |
| tRNA^{Arg} | 112 | GCCCCAGTGGCCTAATGGATAAGGCATTGGCCTGCAA |
| GCA8 | | AGCCAGGGATTGTGGGTTCGAGTCCCATCTGGGGTGcca |
| tRNA^{Arg} | 113 | GCCCCAGTGGCCTGATGGATAAGGTACTGGCCTGCAA |
| GCA9 | | AGCCAGGGATTGTGGGTTCGAGTTCCACCTGGGGTAcca |
| tRNA^{Arg} | 114 | GGCCGCGTGGCCTAATGGATAAGGCGTCTGACTGCAG |
| GCA10 | | ATCAGAAGATTGCAGGTTCGAGTCCTGCCGCGGTCGcca |
| tRNA^{Arg} | 115 | GACCACGTGGCCTAATGGATAAGGCGTCTGACTGCAG |
| GCA11 | | ATCAGAAGATTGAGGGTTCGAATCCCTCCGTGGTTAcca |
| tRNA^{Arg} | 116 | GACCGCGTGGCCTAATGGATAAGGCGTCTGACTGCAG |
| GCA12 | | ATCAGAAGATTGAGGGTTCGAGTCCCTTCGTGGTCGcca |
| tRNA^{Arg} | 117 | GACCACGTGGCCTAATGGATAAGGCGTCTGACTGCAG |
| GCA13 | | ATCAGAAGATTGAGGGTTCGAATCCCTTCGTGGTTAcca |
| tRNA^{Arg} | 118 | GACCACGTGGCCTAATGGATAAGGCGTCTGACTGCAG |
| GCA14 | | ATCAGAAGATTGAGGGTTCGAATCCCTTCGTGGTTGcca |
| tRNA^{Arg} | 119 | GGCCGTGTGGCCTAATGGATAAGGCGTCTGACTGCAG |
| GCA15 | | ATCAAAAGATTGCAGGTTTGAGTTCTGCCACGGTCGcca |
| tRNA^{Arg} | 120 | GGCTCCGTGGCGCAATGGATAGCGCATTGGACTGCAA |
| GCA16 | | ATTCAAAGGTTCCGGGTTCGAGTCCCGGCGGAGTCGcca |
| tRNA^{Arg} | 121 | GGCTCTGTGGCGCAATGGATAGCGCATTGGACTGCAA |
| GCA17 | | ATTCAAAGGTTGTGGGTTCGAATCCCACCAGAGTCGcca |
| tRNA^{Arg} | 122 | GGCTCTGTGGCGCAATGGATAGCGCATTGGACTGCAA |
| GCA18 | | ATTCAAAGGTTGTGGGTTCGAGTCCCACCAGAGTCGcca |
| tRNA^{Arg} | 123 | GTCTCTGTGGCGCAATGGACGAGCGCGCTGGACTGCA |
| | | AATCCAGAGGTTCCGGGTTCGAGTCCCGGCAGAGATGc |
| GCA19 | | ca |
| tRNA^{Arg} | 124 | GGCTCTGTGGCGCAATGGATAGCGCATTGGACTGCAA |
| GCA20 | | ATTCAAAGGTTGCGGGTTCGAGTCCCTCCAGAGTCGcca |
| tRNA^{Arg} | 125 | GGCTCCGTGGCGCAATGGATAGCGCATTGGACTGCAA |
| GCA21 | | GAGGCTGAAGGCATTCAAAGGTTCCGGGTTCGAGTCCC |
| | | GGCGGAGTCGcca |
| tRNA^{Arg} | 126 | GGCTCTGTGGCGCAATGGATAGCGCATTGGACTGCAA |
| GCA22 | | GTGACGAATAGAGCAATTCAAAGGTTGTGGGTTCGAA |
| | | TCCCACCAGAGTCGcca |
| tRNA^{Arg} | 127 | GGCTCTGTGGCGCAATGGATAGCGCATTGGACTGCAA |
| GCA23 | | GCTGAGCCTAGTGTGGTCATTCAAAGGTTGTGGGTTCG |
| | | AGTCCCACCAGAGTCGcca |
| tRNA^{Arg} | 128 | GGCTCTGTGGCGCAATGGATAGCGCATTGGACTGCAA |
| GCA24 | | GATAGTTAGAGAAATTCAAAGGTTGTGGGTTCGAGTCC |
| | | CACCAGAGTCGcca |
| tRNA^{Arg} | 129 | GTCTCTGTGGCGCAATGGACGAGCGCGCTGGACTGCA |
| GCA25 | | AATCCAGAGGTTCCGGGTTCGAGTCCCGGCAGAGATGc |
| | | ca |
| tRNA^{Arg} | 130 | GGCTCTGTGGCGCAATGGATAGCGCATTGGACTGCAA |
| GCA26 | | GCCTAAATCAAGAGATTCAAAGGTTGCGGGTTCGAGTC |
| | | CCTCCAGAGTCGcca |
| tRNA^{Ser} | 131 | GTAGTCGTGGCCGAGTGGTTAAGGCGATGGACTCCGA |
| CCG-01 | | ATCCATTGGGGTTTCCCCGCGCAGGTTCGAATCCTGCC |
| (tRNA1) | | GACTACGcca |
| tRNA^{Ser} | 132 | GCTGTGATGGCCGAGTGGTTAAGGCGTTGGACTCCGAA |
| CCG-02 | | TCCAATGGGGTCTCCCCGCGCAGGTTCGAATCCTGCTC |
| (tRNA2) | | ACAGCGcca |
| tRNA^{Ser} | 133 | GCTGTGATGGCCGAGTGGTTAAGGCGTTGGACTCCGAA |
| CCG-02+ | | TCCAATGGGGTCCACTCCCCGCGCAGGTTCGAATCCTG |
| variable loop | | CTCACAGCGcca |
| expansion | | |
| (tRNA2+) | | |
| tRNA^{Ser} | 134 | GCTGTGATGGCCGAGTGGTTAAGGCGTTGGACTCGAA |
| CGA | | ATCCAATGGGGTCTCCCCGCGCAGGTTCGAATCCTGCT |
| (tRNA3) | | CACAGCGcca |
| p2 | 135 | gacggatcgggagatctcccgatcccctatggtgcactctcagtacaatctgctctgatgcc |
| | | gcatagttaagccagtatctgctccctgcttgtgtgttggaggtcgctgagtagtgcgcgag |
| | | caaaatttaagctacaacaaggcaaggcttgaccgacaattgcatgaagaatctgcttagg |
| | | gttaggcgttttgcgctgcttcgcgatgtacgggccagatatacgcgttgacattgattattga |
| | | ctagttattaatagtaatcaattacggggtcattagttcatagcccatatatggagttccgcgtt |
| | | acataacttacggtaaatggcccgcctggctgaccgcccaacgacccccgcccattgacg |
| | | tcaataatgacgtatgttcccatagtaacgccaatagggactttccattgacgtcaatgggtg |
| | | gagtatttacggtaaactgcccacttggcagtacatcaagtgtatcatatgccaagtacgccc |
| | | cctattgacgtcaatgacggtaaatggcccgcctggcattatgcccagtacatgaccttatg |
| | | ggactttcctacttggcagtacatctacgtattagtcatcgctattaccatggtgatgcggtttt |
| | | ggcagtacatcaatgggcgtggatagcggtttgactcacggggatttccaagtctccaccc |
| | | cattgacgtcaatgggagtttgttttggcaccaaaatcaacgggactttccaaaatgtcgtaa |
| | | caactccgccccattgacgcaaatgggcggtaggcgtgtacggtgggaggtctatataag |
| | | cagagctctctggctaactagagaacccactgcttactggcttatcgaaattaatacgactca |
| | | ctatagggagacccaagctggctagcgccaccatggtgtctaagggcgaggaactgttca |
| | | ccggcgtggtgcctatcctggtggaactggacggagatgtgaacggccataagtttagcgt |
| | | gtccggcgaaggcgaaggcgacgccacctacggcaagctgaccctgaagttcatctgta |
| | | ccaccggcaaactgcccgtgccatggcctaccctggtgacaacactgacctatggcgttca |
| | | gtgcttcagcagataccccgaccacatgaaacagcacgatttctttaaaagcgccatgcctg |
| | | agggctacgtgcaggagagaacaatcttcttcaaagacgacggaaattacaagaccagag |
| | | ccgaggtgaagttcgagggcgatacactggtcaatagaatcgagctgaagggaatcgact |
| | | ttaaggaagatggcaacatcctgggccacaagctcgagtacaactacaacagccacaacg |
| | | tgtacatcatggctgataagcagaagaacggcatcaaggtgaatttcaagatccggcacaa |
| | | catcgaggacggcagcgtgcaactggccgaccactaccagcagaacacccctattggag |
| | | atggccctgtgctgctgcctgacaaccactacctgtctacacagagcaaactgtccaagga |
| | | ccccaacgagaagcgggaccacatggtcctgctggaattcgtgaccgccgctggcatcac |
| | | actgggaatggacgagctgtacaagggcggcagcggcggatctatggtgtccaagggcg |
| | | aggaagataatatggccatcatcaaggaattcatgcggttcaaagtgcacatggaaggcag |
| | | cgtgaacggccatgagtttgagatcgagggagaaggcgagggtagaccttacgagggca |
| | | cccagaccgccaagctgaaggtgacaaagggcggccctctgccattcgcctgggacattc |
| | | tgagccctcagttcatgtacggctctaaggcttatgtgaagcaccccgctgatatccctgact |
| | | atctgaagctctccttccccgagggcttcaagtgggaacgggtgatgaacttcgaggatgg |
| | | cggagtggtgacagtgacacaagacagcagcctgcaggacggcgaattcatctacaaag |
| | | tgaagctgagaggcaccaattttccatctgatggacctgtgatgcagaaaaagaccatggg |
| | | ctgggaggccagcagcgagagaatgtaccctgaggacggagctctgaagggcgaaatc |
| | | aaacagagactgaagctgaaggacggaggccactacgacgccgaagtgaaaacaacct |
| | | acaaggccaagaagcccgtccagctgcctggcgcctacaacgttaacatcaagctggaca |
| | | tcaccagccacaacgaggattacaccatcgtggaacagtacgagcgcgccgaaggaaga |
| | | cacagcaccggcggcatggacgagctgtacaagtgaaagcttggtaccgagctcggatc |
| | | cactagtccagtgtggtggaattctgcagatatccagcacagtggcggccgctcgagtcta |
| | | gagggcccgtttaaacccgctgatcagcctcgactgtgccttctagttgccagccatctgttg |
| | | tttgcccctcccccgtgccttccttgaccctggaaggtgccactcccactgtcctttcctaata |
| | | aaatgaggaaattgcatcgcattgtctgagtaggtgtcattctattctgggggggggggg |
| | | ggcaggacagcaagggggaggattgggaagacaatagcaggcatgctggggatgcggt |
| | | gggctctatggcttctgaggcggaaagaaccagctggggctctagggggtatccccacgc |
| | | gccctgtagcggcgcattaagcgcggcgggtgtggtggttacgcgcagcgtgaccgcta |
| | | cacttgccagcgccctagegcccgctcctttcgctttcttcccttcctttctcgccacgttcgcc |
| | | ggctttccccgtcaagctctaaatcgggggctccctttagggttccgatttagtgctttacggc |
| | | acctcgaccccaaaaaacttgattagggtgatggttcacgtagtgggccatcgccctgatag |
| | | acggtttttcgccctttgacgttggagtccacgttctttaatagtggactcttgttccaaactgg |
| | | aacaacactcaaccctatctcggtctattcttttgatttataagggattttgccgatttcggccta |
| | | ttggttaaaaaatgagctgatttaacaaaaatttaacgcgaattaattctgtggaatgtgtgtca |
| | | gttagggtgtggaaagtccccaggctccccagcaggcagaagtatgcaaagcatgcatct |
| | | caattagtcagcaaccaggtgtggaaagtccccaggctccccagcaggcagaagtatgca |
| | | aagcatgcatctcaattagtcagcaaccatagtcccgcccctaactccgcccatcccgccc |
| | | ctaactccgcccagttccgcccattctccgccccatggctgactaattttttttatttatgcaga |
| | | ggccgaggccgcctctgcctctgagctattccagaagtagtgaggaggcttttttggaggc |
| | | ctaggcttttgcaaaaagctcccgggagcttgtatatccattttcggatctgatcaagagaca |
| | | ggatgaggatcgtttcgcatggtggagtacaagcccacggtgcgcctcgccacccgcgac |
| | | gacgtccccagggccgtacgcaccctcgccgccgegttcgccgactaccccgccacgcg |
| | | ccacaccgtcgatccggaccgccacatcgagcgggtcaccgagctgcaagaactcttcct |
| | | cacgcgcgtcgggctcgacatcggcaaggtgtgggtcgcggacgacggcgccgcggtg |
| | | gcggtctggaccacgccggagagcgtcgaagcgggggcggtgttcgccgagatcggcc |
| | | cgcgcatggccgagttgagcggttcccggctggccgcgcagcaacagatggaaggcctc |
| | | ctggcgccgcaccggcccaaggagcccgcgtggttcctggccaccgtcggcgtctcgcc |
| | | cgaccaccagggcaagggtctgggcagcgccgtcgtgctccccggagtggaggcggcc |
| | | gagcgcgccggggtgcccgccttcctggagacctccgcgccccgcaacctccccttctac |
| | | gagcggctcggcttcaccgtcaccgccgacgtcgaggtgcccgaaggaccgcgcacct |
| | | ggtgcatgacccgcaagcccggtgccgcgggactctggggttcgaaatgaccgaccaag |
| | | cgacgcccaacctgccatcacgagatttcgattccaccgccgccttctatgaaaggttggg |
| | | cttcggaatcgttttccgggacgccggctggatgatcctccagcgcggggatctcatgctg |
| | | gagttcttcgcccaccccaacttgtttattgcagcttataatggttacaaataaagcaatagcat |
| | | cacaaatttcacaaataaagcatttttttcactgcattctagttgtggtttgtccaaactcatcaat |
| | | gtatcttatcatgtctgtataccgtcgacctctagctagagcttggcgtaatcatggtcatagct |
| | | gtttcctgtgtgaaattgttatccgctcacaattccacacaacatacgagccggaagcataaa |
| | | gtgtaaagcctggggtgcctaatgagtgagctaactcacattaattgcgttgcgctcactgcc |
| | | cgctttccagtcgggaaacctgtcgtgccagctgcattaatgaatcggccaacgcgcggg |
| | | gagaggcggtttgcgtattgggcgctcttccgcttcctcgctcactgactcgctgcgctcggt |
| | | cgttcggctgcggcgagcggtatcagctcactcaaaggcggtaatacggttatccacagaa |
| | | tcaggggataacgcaggaaagaacatgtgagcaaaaggccagcaaaaggccaggaacc |
| | | gtaaaaaggccgcgttgctggcgtttttccataggctccgcccccctgacgagcatcacaa |
| | | aaatcgacgctcaagtcagaggtggcgaaacccgacaggactataaagataccaggcgtt |
| | | tccccctggaagctccctcgtgcgctctcctgttccgaccctgccgcttaccggatacctgtc |
| | | cgcctttctcccttcgggaagcgtggcgctttctcatagctcacgctgtaggtatctcagttcg |
| | | gtgtaggtcgttcgctccaagctgggctgtgtgcacgaaccccccgttcagcccgaccgct |
| | | gcgccttatccggtaactatcgtcttgagtccaacccggtaagacacgacttatcgccactg |
| | | gcagcagccactggtaacaggattagcagagcgaggtatgtaggcggtgctacagagttc |
| | | ttgaagtggtggcctaactacggctacactagaagaacagtatttggtatctgcgctctgctg |
| | | aagccagttaccttcggaaaaagagttggtagctcttgatccggcaaacaaaccaccgctg |
| | | gtagcggtggtttttttgtttgcaagcagcagattacgcgcagaaaaaaaggatctcaagaa |
| | | gatcctttgatcttttctacggggtctgacgctcagtggaacgaaaactcacgttaagggattt |
| | | tggtcatgagattatcaaaaaggatcttcacctagatccttttaaattaaaaatgaagttttaaat |
| | | caatctaaagtatatatgagtaaacttggtctgacagttaccaatgcttaatcagtgaggcacc |
| | | tatctcagcgatctgtctatttcgttcatccatagttgcctgactccccgtcgtgtagataactac |
| | | gatacgggagggcttaccatctggccccagtgctgcaatgataccgcgagacccacgctc |
| | | accggctccagatttatcagcaataaaccagccagccggaagggccgagcgcagaagtg |
| | | gtcctgcaactttatccgcctccatccagtctattaattgttgccgggaagctagagtaagtag |
| | | ttcgccagttaatagtttgcgcaacgttgttgccattgctacaggcatcgtggtgtcacgctcg |
| | | tcgtttggtatggcttcattcagctccggttcccaacgatcaaggegagttacatgatccccc |
| | | atgttgtgcaaaaaagcggttagctccttcggtcctccgatcgttgtcagaagtaagttggcc |
| | | gcagtgttatcactcatggttatggcagcactgcataattctcttactgtcatgccatccgtaag |
| | | atgcttttctgtgactggtgagtactcaaccaagtcattctgagaatagtgtatgcggcgacc |
| | | gagttgctcttgcccggcgtcaatacgggataataccgcgccacatagcagaactttaaaa |
| | | gtgctcatcattggaaaacgttcttcggggcgaaaactctcaaggatcttaccgctgttgaga |
| | | tccagttcgatgtaacccactcgtgcacccaactgatcttcagcatcttttactttcaccagcgt |
| | | ttctgggtgagcaaaaacaggaaggcaaaatgccgcaaaaaagggaataagggcgaca |
| | | cggaaatgttgaatactcatactcttcctttttcaatattattgaagcatttatcagggttattgtct |
| | | catgagcggatacatatttgaatgtatttagaaaaataaacaaataggggttccgcgcacatt |
| | | tccccgaaaagtgccacctgacgtc |
| p100 | 136 | gacggatcgggagatctcccgatcccctatggtgcactctcagtacaatctgctctgatgcc |
| | | gcatagttaagccagtatctgctccctgcttgtgtgttggaggtcgctgagtagtgcgcgag |
| | | caaaatttaagctacaacaaggcaaggcttgaccgacaattgcatgaagaatctgcttagg |
| | | gttaggcgttttgcgctgcttcgcgatgtacgggccagatatacgcgttgacattgattattga |
| | | ctagttattaatagtaatcaattacggggtcattagttcatagcccatatatggagttccgcgtt |
| | | acataacttacggtaaatggcccgcctggctgaccgcccaacgacccccgcccattgacg |
| | | tcaataatgacgtatgttcccatagtaacgccaatagggactttccattgacgtcaatgggtg |
| | | gagtatttacggtaaactgcccacttggcagtacatcaagtgtatcatatgccaagtacgccc |
| | | cctattgacgtcaatgacggtaaatggcccgcctggcattatgcccagtacatgaccttatg |
| | | ggactttcctacttggcagtacatctacgtattagtcatcgctattaccatggtgatgcggtttt |
| | | ggcagtacatcaatgggcgtggatagcggtttgactcacggggatttccaagtctccaccc |
| | | cattgacgtcaatgggagtttgttttggcaccaaaatcaacgggactttccaaaatgtcgtaa |
| | | caactccgccccattgacgcaaatgggcggtaggcgtgtacggtgggaggtctatataag |
| | | cagagctctctggctaactagagaacccactgcttactggcttatcgaaattaatacgactca |
| | | ctatagggagacccaagctggctagcgccaccatggtgtctaagggcgaggaactgttca |
| | | ccggcgtggtgcctatcctggtggaactggacggagatgtgaacggccataagtttagcgt |
| | | gtccggcgaaggcgaaggcgacgccacctacggcaagctgaccctgaagttcatctgta |
| | | ccaccggcaaactgcccgtgccatggcctaccctggtgacaacactgacctatggcgttca |
| | | gtgcttcagcagataccccgaccacatgaaacagcacgatttctttaaaagcgccatgcctg |
| | | agggctacgtgcaggagCAGacaatcttcttcaaagacgacggaaattacaagaccag |
| | | agccgaggtgaagttcgagggcgatacactggtcaatagaatcgagctgaagggaatcg |
| | | actttaaggaagatggcaacatcctgggccacaagctcgagtacaactacaacagccaca |
| | | acgtgtacatcatggctgataagcagaagaacggcatcaaggtgaatttcaagatccggca |
| | | caacatcgaggacggcagcgtgcaactggccgaccactaccagcagaacacccctattg |
| | | gagatggccctgtgctgctgcctgacaaccactacctgtctacacagagcaaactgtccaa |
| | | ggaccccaacgagaagcgggaccacatggtcctgctggaattcgtgaccgccgctggca |
| | | tcacactgggaatggacgagctgtacaagggcggcagcggcggatctatggtgtccaag |
| | | ggcgaggaagataatatggccatcatcaaggaattcatgcggttcaaagtgcacatggaag |
| | | gcagcgtgaacggccatgagtttgagatcgagggagaaggcgagggtagaccttacgag |
| | | ggcacccagaccgccaagctgaaggtgacaaagggcggccctctgccattcgcctggga |
| | | cattctgagccctcagttcatgtacggctctaaggcttatgtgaagcaccccgctgatatccc |
| | | tgactatctgaagctctccttccccgagggcttcaagtgggaacgggtgatgaacttcgagg |
| | | atggcggagtggtgacagtgacacaagacagcagcctgcaggacggcgaattcatctac |
| | | aaagtgaagctgagaggcaccaattttccatctgatggacctgtgatgcagaaaaagacca |
| | | tgggctgggaggccagcagcgagagaatgtaccctgaggacggagctctgaagggcga |
| | | aatcaaacagagactgaagctgaaggacggaggccactacgacgccgaagtgaaaaca |
| | | acctacaaggccaagaagcccgtccagctgcctggcgcctacaacgttaacatcaagctg |
| | | gacatcaccagccacaacgaggattacaccatcgtggaacagtacgagcgcgccgaagg |
| | | aagacacagcaccggcggcatggacgagctgtacaagtgaaagcttggtaccgagctcg |
| | | gatccactagtccagtgtggtggaattctgcagatatccagcacagtggcggccgctcgag |
| | | tctagagggcccgtttaaacccgctgatcagcctcgactgtgccttctagttgccagccatct |
| | | gttgtttgcccctcccccgtgccttccttgaccctggaaggtgccactcccactgtcctttcct |
| | | aataaaatgaggaaattgcatcgcattgtctgagtaggtgtcattctattctggggggtgggg |
| | | tggggcaggacagcaagggggaggattgggaagacaatagcaggcatgctggggatgc |
| | | ggtgggctctatggcttctgaggcggaaagaaccagctggggctctagggggtatcccca |
| | | cgcgccctgtagcggcgcattaagcgcggcgggtgtggtggttacgcgcagcgtgaccg |
| | | ctacacttgccagcgccctagegcccgctcctttcgctttcttcccttcctttctcgccacgttc |
| | | gccggctttccccgtcaagctctaaatcgggggctccctttagggttccgatttagtgctttac |
| | | ggcacctcgaccccaaaaaacttgattagggtgatggttcacgtagtgggccatcgccctg |
| | | atagacggtttttcgccctttgacgttggagtccacgttctttaatagtggactcttgttccaaac |
| | | tggaacaacactcaaccctatctcggtctattcttttgatttataagggattttgccgatttcggc |
| | | ctattggttaaaaaatgagctgatttaacaaaaatttaacgcgaattaattctgtggaatgtgtg |
| | | tcagttagggtgtggaaagtccccaggctccccagcaggcagaagtatgcaaagcatgca |
| | | tctcaattagtcagcaaccaggtgtggaaagtccccaggctccccagcaggcagaagtat |
| | | gcaaagcatgcatctcaattagtcagcaaccatagtcccgcccctaactccgcccatcccg |
| | | cccctaactccgcccagttccgcccattctccgccccatggctgactaattttttttatttatgca |
| | | gaggccgaggccgcctctgcctctgagctattccagaagtagtgaggaggcttttttggag |
| | | gcctaggcttttgcaaaaagctcccgggagcttgtatatccattttcggatctgatcaagaga |
| | | caggatgaggatcgtttcgcatggtggagtacaagcccacggtgcgcctcgccacccgcg |
| | | acgacgtccccagggccgtacgcaccctcgccgccgegttcgccgactaccccgccacg |
| | | cgccacaccgtcgatccggaccgccacatcgaggggtcaccgagctgcaagaactctt |
| | | cctcacgcgcgtcgggctcgacatcggcaaggtgtgggtcgcggacgacggcgccgcg |
| | | gtggcggtctggaccacgccggagagcgtcgaagcgggggcggtgttcgccgagatcg |
| | | gcccgcgcatggccgagttgagcggttcccggctggccgcgcagcaacagatggaagg |
| | | cctcctggcgccgcaccggcccaaggagcccgcgtggttcctggccaccgtcggcgtct |
| | | cgcccgaccaccagggcaagggtctgggcagcgccgtcgtgctccccggagtggaggc |
| | | ggccgagcgcgccggggtgcccgccttcctggagacctccgcgccccgcaacctcccct |
| | | tctacgagcggctcggcttcaccgtcaccgccgacgtcgaggtgcccgaaggaccgcgc |
| | | acctggtgcatgacccgcaagcccggtgccgcgggactctggggttcgaaatgaccgac |
| | | caagcgacgcccaacctgccatcacgagatttcgattccaccgccgccttctatgaaaggtt |
| | | gggcttcggaatcgttttccgggacgccggctggatgatcctccagcgcggggatctcatg |
| | | ctggagttcttcgcccaccccaacttgtttattgcagcttataatggttacaaataaagcaata |
| | | gcatcacaaatttcacaaataaagcatttttttcactgcattctagttgtggtttgtccaaactcat |
| | | caatgtatcttatcatgtctgtataccgtcgacctctagctagagcttggcgtaatcatggtcat |
| | | agctgtttcctgtgtgaaattgttatccgctcacaattccacacaacatacgagccggaagca |
| | | taaagtgtaaagcctggggtgcctaatgagtgagctaactcacattaattgcgttgcgctcac |
| | | tgcccgctttccagtcgggaaacctgtcgtgccagctgcattaatgaatcggccaacgcgc |
| | | ggggagaggcggtttgcgtattgggcgctcttccgcttcctcgctcactgactcgctgcgct |
| | | cggtcgttcggctgcggcgagcggtatcagctcactcaaaggcggtaatacggttatccac |
| | | agaatcaggggataacgcaggaaagaacatgtgagcaaaaggccagcaaaaggccag |
| | | gaaccgtaaaaaggccgcgttgctggcgtttttccataggctccgcccccctgacgagcat |
| | | cacaaaaatcgacgctcaagtcagaggtggcgaaacccgacaggactataaagatacca |
| | | ggcgtttccccctggaagctccctcgtgcgctctcctgttccgaccctgccgcttaccggat |
| | | acctgtccgcctttctcccttcgggaagcgtggcgctttctcatagctcacgctgtaggtatct |
| | | cagttcggtgtaggtcgttcgctccaagctgggctgtgtgcacgaaccccccgttcagccc |
| | | gaccgctgcgccttatccggtaactatcgtcttgagtccaacccggtaagacacgacttatc |
| | | gccactggcagcagccactggtaacaggattagcagagcgaggtatgtaggcggtgcta |
| | | cagagttcttgaagtggtggcctaactacggctacactagaagaacagtatttggtatctgcg |
| | | ctctgctgaagccagttaccttcggaaaaagagttggtagctcttgatccggcaaacaaacc |
| | | accgctggtagcggtggtttttttgtttgcaagcagcagattacgcgcagaaaaaaaggatc |
| | | tcaagaagatcctttgatcttttctacggggtctgacgctcagtggaacgaaaactcacgtta |
| | | agggattttggtcatgagattatcaaaaaggatcttcacctagatccttttaaattaaaaatgaa |
| | | gttttaaatcaatctaaagtatatatgagtaaacttggtctgacagttaccaatgcttaatcagtg |
| | | aggcacctatctcagcgatctgtctatttcgttcatccatagttgcctgactccccgtcgtgtag |
| | | ataactacgatacgggagggcttaccatctggccccagtgctgcaatgataccgcgagac |
| | | ccacgctcaccggctccagatttatcagcaataaaccagccagccggaagggccgagcg |
| | | cagaagtggtcctgcaactttatccgcctccatccagtctattaattgttgccgggaagctag |
| | | agtaagtagttcgccagttaatagtttgcgcaacgttgttgccattgctacaggcatcgtggtg |
| | | tcacgctcgtcgtttggtatggcttcattcagctccggttcccaacgatcaaggcgagttacat |
| | | gatcccccatgttgtgcaaaaaagcggttagctccttcggtcctccgatcgttgtcagaagta |
| | | agttggccgcagtgttatcactcatggttatggcagcactgcataattctcttactgtcatgcc |
| | | atccgtaagatgcttttctgtgactggtgagtactcaaccaagtcattctgagaatagtgtatg |
| | | cggcgaccgagttgctcttgcccggcgtcaatacgggataataccgcgccacatagcaga |
| | | actttaaaagtgctcatcattggaaaacgttcttcggggcgaaaactctcaaggatcttaccg |
| | | ctgttgagatccagttcgatgtaacccactcgtgcacccaactgatcttcagcatcttttacttt |
| | | caccagcgtttctgggtgagcaaaaacaggaaggcaaaatgccgcaaaaaagggaataa |
| | | gggcgacacggaaatgttgaatactcatactcttcctttttcaatattattgaagcatttatcag |
| | | ggttattgtctcatgagcggatacatatttgaatgtatttagaaaaataaacaaataggggttc |
| | | cgcgcacatttccccgaaaagtgccacctgacgtc |
| p108 | 137 | gacggatcgggagatctcccgatcccctatggtgcactctcagtacaatctgctctgatgcc |
| | | gcatagttaagccagtatctgctccctgcttgtgtgttggaggtcgctgagtagtgcgcgag |
| | | caaaatttaagctacaacaaggcaaggcttgaccgacaattgcatgaagaatctgcttagg |
| | | gttaggcgttttgcgctgcttcgcgatgtacgggccagatatacgcgttgacattgattattga |
| | | ctagttattaatagtaatcaattacggggtcattagttcatagcccatatatggagttccgcgtt |
| | | acataacttacggtaaatggcccgcctggctgaccgcccaacgacccccgcccattgacg |
| | | tcaataatgacgtatgttcccatagtaacgccaatagggactttccattgacgtcaatgggtg |
| | | gagtatttacggtaaactgcccacttggcagtacatcaagtgtatcatatgccaagtacgccc |
| | | cctattgacgtcaatgacggtaaatggcccgcctggcattatgcccagtacatgaccttatg |
| | | ggactttcctacttggcagtacatctacgtattagtcatcgctattaccatggtgatgcggtttt |
| | | ggcagtacatcaatgggcgtggatagcggtttgactcacggggatttccaagtctccaccc |
| | | cattgacgtcaatgggagtttgttttggcaccaaaatcaacgggactttccaaaatgtcgtaa |
| | | caactccgccccattgacgcaaatgggcggtaggcgtgtacggtgggaggtctatataag |
| | | cagagctctctggctaactagagaacccactgcttactggcttatcgaaattaatacgactca |
| | | ctatagggagacccaagctggctagcgccaccatggtgtctaagggcgaggaactgttca |
| | | ccggcgtggtgcctatcctggtggaactggacggagatgtgaacggccataagtttagcgt |
| | | gtccggcgaaggcgaaggcgacgccacctacggcaagctgaccctgaagttcatctgta |
| | | ccaccggcaaactgcccgtgccatggcctaccctggtgacaacactgacctatggcgttca |
| | | gtgcttcagcagataccccgaccacatgaaacagcacgatttctttaaaagcgccatgcctg |
| | | agggctacgtgcaggagCAGacaatcttcttcaaagacgacggaaattacaagaccag |
| | | agccgaggtgaagttcgagggcgatacactggtcaatagaatcgagctgaagggaatcg |
| | | actttaaggaagatggcaacatcctgggccacaagctcgagtacaactacaacagccaca |
| | | acgtgtacatcatggctgataagcagaagaacggcatcaaggtgaatttcaagatccggca |
| | | caacatcgaggacggcagcgtgcaactggccgaccactaccagcagaacacccctattg |
| | | gagatggccctgtgctgctgcctgacaaccactacctgtctacacagagcaaactgtccaa |
| | | ggaccccaacgagaagcgggaccacatggtcctgctggaattcgtgaccgccgctggca |
| | | tcacactgggaatggacgagctgtacaagggcggcagcggcggatctatggtgtccaag |
| | | ggcgaggaagataatatggccatcatcaaggaattcatgcggttcaaagtgcacatggaag |
| | | gcagcgtgaacggccatgagtttgagatcgagggagaaggcgagggtagaccttacgag |
| | | ggcacccagaccgccaagctgaaggtgacaaagggcggccctctgccattcgcctggga |
| | | cattctgagccctcagttcatgtacggctctaaggcttatgtgaagcaccccgctgatatccc |
| | | tgactatctgaagctctccttccccgagggcttcaagtgggaacgggtgatgaacttcgagg |
| | | atggcggagtggtgacagtgacacaagacagcagcctgcaggacggcgaattcatctac |
| | | aaagtgaagctgagaggcaccaattttccatctgatggacctgtgatgcagaaaaagacca |
| | | tgggctgggaggccagcagcgagagaatgtaccctgaggacggagctctgaagggcga |
| | | aatcaaacagagactgaagctgaaggacggaggccactacgacgccgaagtgaaaaca |
| | | acctacaaggccaagaagcccgtccagctgcctggcgcctacaacgttaacatcaagctg |
| | | gacatcaccagccacaacgaggattacaccatcgtggaacagtacgagcgcgccgaagg |
| | | aagacacagcaccggcggcatggacgagctgtacaagtgaaagcttggtaccgagctcg |
| | | gatccactagtccagtgtggtggaattctgcagatatccagcacagtggcggccgctcgag |
| | | tctagagggcccgtttaaacccgctgatcagcctcgactgtgccttctagttgccagccatct |
| | | gttgtttgcccctcccccgtgccttccttgaccctggaaggtgccactcccactgtcctttcct |
| | | aataaaatgaggaaattgcatcgcattgtctgagtaggtgtcattctattctggggggtgggg |
| | | tggggcaggacagcaagggggaggattgggaagacaatagcaggcatgctggggatgc |
| | | ggtgggctctatggcttctgaggcggaaagaaccagctggggctctagggggtatcccca |
| | | cgcgccctgtagcggcgcattaagcgcggcgggtgtggtggttacgcgcagcgtgaccg |
| | | ctacacttgccagcgccctagegcccgctcctttcgctttcttcccttcctttctcgccacgttc |
| | | gccggctttccccgtcaagctctaaatcgggggctccctttagggttccgatttagtgctttac |
| | | ggcacctcgaccccaaaaaacttgattagggtgatggttcacgtagtgggccatcgccctg |
| | | atagacggtttttcgccctttgacgttggagtccacgttctttaatagtggactcttgttccaaac |
| | | tggaacaacactcaaccctatctcggtctattcttttgatttataagggattttgccgatttcggc |
| | | ctattggttaaaaaatgagctgatttaacaaaaatttaacgcgaattaattctgtggaatgtgtg |
| | | tcagttagggtgtggaaagtccccaggctccccagcaggcagaagtatgcaaagcatgca |
| | | tctcaattagtcagcaaccaggtgtggaaagtccccaggctccccagcaggcagaagtat |
| | | gcaaagcatgcatctcaattagtcagcaaccatagtcccgcccctaactccgcccatcccg |
| | | cccctaactccgcccagttccgcccattctccgccccatggctgactaattttttttatttatgca |
| | | gaggccgaggccgcctctgcctctgagctattccagaagtagtgaggaggcttttttggag |
| | | gcctaggcttttgcaaaaagctcccgggagcttgtatatccattttcggatctgatcaagaga |
| | | caggatgaggatcgtttcgcatggtggagtacaagcccacggtgcgcctcgccacccgcg |
| | | acgacgtccccagggccgtacgcaccctcgccgccgegttcgccgactaccccgccacg |
| | | cgccacaccgtcgatccggaccgccacatcgagcgggtcaccgagctgcaagaactctt |
| | | cctcacgcgcgtcgggctcgacatcggcaaggtgtgggtcgcggacgacggcgccgcg |
| | | gtggcggtctggaccacgccggagagcgtcgaagcgggggcggtgttcgccgagatcg |
| | | gcccgcgcatggccgagttgagcggttcccggctggccgcgcagcaacagatggaagg |
| | | cctcctggcgccgcaccggcccaaggagcccgcgtggttcctggccaccgtcggcgtct |
| | | cgcccgaccaccagggcaagggtctgggcagcgccgtcgtgctccccggagtggaggc |
| | | ggccgagcgcgccggggtgcccgccttcctggagacctccgegccccgcaacctcccct |
| | | tctacgagcggctcggcttcaccgtcaccgccgacgtcgaggtgcccgaaggaccgcgc |
| | | acctggtgcatgacccgcaagcccggtgccgcgggactctggggttcgaaatgaccgac |
| | | caagcgacgcccaacctgccatcacgagatttcgattccaccgccgccttctatgaaaggtt |
| | | gggcttcggaatcgttttccgggacgccggctggatgatcctccagcgcggggatctcatg |
| | | ctggagttcttcgcccaccccaacttgtttattgcagcttataatggttacaaataaagcaata |
| | | gcatcacaaatttcacaaataaagcatttttttcactgcattctagttgtggtttgtccaaactcat |
| | | caatgtatcttatcatgtctgtataccgtcgacctctagctagagcttggcgtaatcatggtcat |
| | | agctgtttcctgtgtgaaattgttatccgctcacaattccacacaacatacgagccggaagca |
| | | taaagtgtaaagcctggggtgcctaatgagtgagctaactcacattaattgcgttgcgctcac |
| | | tgcccgctttccagtcgggaaacctgtcgtgccagctgcattaatgaatcggccaacgcgc |
| | | ggggagaggcggtttgcgtattgggcgctcttccCATCGGTGCAGTTTCGA |
| | | GGGTAAAGCCTTTGGCGCGGTGATGTGGACTTTTGTTC |
| | | TCTAACTACAACTCCCAGCATACGTCACCCCTCACGTG |
| | | GGCGCTAGGTGTGGTTTCGTGGGATAGGGTAAGGCGA |
| | | AAATGAAGTGACGATGACGTACCAAAGAGGAAAATAG |
| | | TTGAATATGTTGTGTGAGCGCGTCGTTTGCAGAAGCCC |
| | | CAGTGGCCTAATGGATAAGGCATTGGCCTCTGAAGCCA |
| | | GGGATTGTGGGTTCGAGTCCCATCTGGGGTGGCCTGTG |
| | | ACTTTtgtccttttttcccctttctaaaCAAAGCGAAATATgccaactttacttt |
| | | ttttttttttaaatgggaagtgtgtgtgtgtttttactaGCTGGTGGGCTGCAATT |
| | | TTCGAGAATCTGCACACACCCATGTCCTTTGTCTGAGC |
| | | CGCAGGTCCCCTGGGATTTGGGGAGACGTCAATACGTT |
| | | GAATTTgcttcctcgctcactgactcgctgcgctcggtcgttcggctgcggcgagcgg |
| | | tatcagctcactcaaaggcggtaatacggttatccacagaatcaggggataacgcaggaaa |
| | | gaacatgtgagcaaaaggccagcaaaaggccaggaaccgtaaaaaggccgcgttgctg |
| | | gcgtttttccataggctccgcccccctgacgagcatcacaaaaatcgacgctcaagtcaga |
| | | ggtggcgaaacccgacaggactataaagataccaggcgtttccccctggaagctccctcg |
| | | tgcgctctcctgttccgaccctgccgcttaccggatacctgtccgcctttctcccttcgggaa |
| | | gcgtggcgctttctcatagctcacgctgtaggtatctcagttcggtgtaggtcgttcgctccaa |
| | | gctgggctgtgtgcacgaaccccccgttcagcccgaccgctgcgccttatccggtaactat |
| | | cgtcttgagtccaacccggtaagacacgacttatcgccactggcagcagccactggtaaca |
| | | ggattagcagagcgaggtatgtaggcggtgctacagagttcttgaagtggtggcctaacta |
| | | cggctacactagaagaacagtatttggtatctgcgctctgctgaagccagttaccttcggaa |
| | | aaagagttggtagctcttgatccggcaaacaaaccaccgctggtagcggtggtttttttgtttg |
| | | caagcagcagattacgcgcagaaaaaaaggatctcaagaagatcctttgatcttttctacgg |
| | | ggtctgacgctcagtggaacgaaaactcacgttaagggattttggtcatgagattatcaaaa |
| | | aggatcttcacctagatccttttaaattaaaaatgaagttttaaatcaatctaaagtatatatgag |
| | | taaacttggtctgacagttaccaatgcttaatcagtgaggcacctatctcagcgatctgtctatt |
| | | tcgttcatccatagttgcctgactccccgtcgtgtagataactacgatacgggagggcttacc |
| | | atctggccccagtgctgcaatgataccgcgagacccacgctcaccggctccagatttatca |
| | | gcaataaaccagccagccggaagggccgagcgcagaagtggtcctgcaactttatccgc |
| | | ctccatccagtctattaattgttgccgggaagctagagtaagtagttcgccagttaatagtttg |
| | | cgcaacgttgttgccattgctacaggcatcgtggtgtcacgctcgtcgtttggtatggcttcat |
| | | tcagctccggttcccaacgatcaaggcgagttacatgatcccccatgttgtgcaaaaaagc |
| | | ggttagctccttcggtcctccgatcgttgtcagaagtaagttggccgcagtgttatcactcatg |
| | | gttatggcagcactgcataattctcttactgtcatgccatccgtaagatgcttttctgtgactggt |
| | | gagtactcaaccaagtcattctgagaatagtgtatgcggcgaccgagttgctcttgcccggc |
| | | gtcaatacgggataataccgcgccacatagcagaactttaaaagtgctcatcattggaaaac |
| | | gttcttcggggcgaaaactctcaaggatcttaccgctgttgagatccagttcgatgtaaccca |
| | | ctcgtgcacccaactgatcttcagcatcttttactttcaccagcgtttctgggtgagcaaaaac |
| | | aggaaggcaaaatgccgcaaaaaagggaataagggcgacacggaaatgttgaatactca |
| | | tactcttcctttttcaatattattgaagcatttatcagggttattgtctcatgagcggatacatattt |
| | | gaatgtatttagaaaaataaacaaataggggttccgcgcacatttccccgaaaagtgccacc |
| | | tgacgtc |
| p401 | 138 | gacggatcgggagatctcccgatcccctatggtgcactctcagtacaatctgctctgatgcc |
| | | gcatagttaagccagtatctgctccctgcttgtgtgttggaggtcgctgagtagtgcgcgag |
| | | caaaatttaagctacaacaaggcaaggcttgaccgacaattgcatgaagaatctgcttagg |
| | | gttaggcgttttgcgctgcttcgcgatgtacgggccagatatacgcgttgacattgattattga |
| | | ctagttattaatagtaatcaattacggggtcattagttcatagcccatatatggagttccgcgtt |
| | | acataacttacggtaaatggcccgcctggctgaccgcccaacgacccccgcccattgacg |
| | | tcaataatgacgtatgttcccatagtaacgccaatagggactttccattgacgtcaatgggtg |
| | | gagtatttacggtaaactgcccacttggcagtacatcaagtgtatcatatgccaagtacgccc |
| | | cctattgacgtcaatgacggtaaatggcccgcctggcattatgcccagtacatgaccttatg |
| | | ggactttcctacttggcagtacatctacgtattagtcatcgctattaccatggtgatgcggtttt |
| | | ggcagtacatcaatgggcgtggatagcggtttgactcacggggatttccaagtctccaccc |
| | | cattgacgtcaatgggagtttgttttggcaccaaaatcaacgggactttccaaaatgtcgtaa |
| | | caactccgccccattgacgcaaatgggcggtaggcgtgtacggtgggaggtctatataag |
| | | cagagctctctggctaactagagaacccactgcttactggcttatcgaaattaatacgactca |
| | | ctatagggagacccaagctggctagcgccaccatggtgtctaagggcgaggaactgttca |
| | | ccggcgtggtgcctatcctggtggaactggacggagatgtgaacggccataagtttagcgt |
| | | gtccggcgaaggcgaaggcgacgccacctacggcaagctgaccctgaagttcatctgta |
| | | ccaccggcaaactgcccgtgccatggcctaccctggtgacaacactgacctatggcgttca |
| | | gtgcttcagcagataccccgaccacatgaaacagcacgatttctttaaaagcgccatgcctg |
| | | agggctacgtgcaggagTGCacaatcttcttcaaagacgacggaaattacaagaccag |
| | | agccgaggtgaagttcgagggcgatacactggtcaatagaatcgagctgaagggaatcg |
| | | actttaaggaagatggcaacatcctgggccacaagctcgagtacaactacaacagccaca |
| | | acgtgtacatcatggctgataagcagaagaacggcatcaaggtgaatttcaagatccggca |
| | | caacatcgaggacggcagcgtgcaactggccgaccactaccagcagaacacccctattg |
| | | gagatggccctgtgctgctgcctgacaaccactacctgtctacacagagcaaactgtccaa |
| | | ggaccccaacgagaagcgggaccacatggtcctgctggaattcgtgaccgccgctggca |
| | | tcacactgggaatggacgagctgtacaagggcggcagcggcggatctatggtgtccaag |
| | | ggcgaggaagataatatggccatcatcaaggaattcatgcggttcaaagtgcacatggaag |
| | | gcagcgtgaacggccatgagtttgagatcgagggagaaggcgagggtagaccttacgag |
| | | ggcacccagaccgccaagctgaaggtgacaaagggcggccctctgccattcgcctggga |
| | | cattctgagccctcagttcatgtacggctctaaggcttatgtgaagcaccccgctgatatccc |
| | | tgactatctgaagctctccttccccgagggcttcaagtgggaacgggtgatgaacttcgagg |
| | | atggcggagtggtgacagtgacacaagacagcagcctgcaggacggcgaattcatctac |
| | | aaagtgaagctgagaggcaccaattttccatctgatggacctgtgatgcagaaaaagacca |
| | | tgggctgggaggccagcagcgagagaatgtaccctgaggacggagctctgaagggcga |
| | | aatcaaacagagactgaagctgaaggacggaggccactacgacgccgaagtgaaaaca |
| | | acctacaaggccaagaagcccgtccagctgcctggcgcctacaacgttaacatcaagctg |
| | | gacatcaccagccacaacgaggattacaccatcgtggaacagtacgagcgcgccgaagg |
| | | aagacacagcaccggcggcatggacgagctgtacaagtgaaagcttggtaccgagctcg |
| | | gatccactagtccagtgtggtggaattctgcagatatccagcacagtggcggccgctcgag |
| | | tctagagggcccgtttaaacccgctgatcagcctcgactgtgccttctagttgccagccatct |
| | | gttgtttgcccctcccccgtgccttccttgaccctggaaggtgccactcccactgtcctttcct |
| | | aataaaatgaggaaattgcatcgcattgtctgagtaggtgtcattctattctggggggtgggg |
| | | tggggcaggacagcaagggggaggattgggaagacaatagcaggcatgctggggatgc |
| | | ggtgggctctatggcttctgaggcggaaagaaccagctggggctctagggggtatcccca |
| | | cgcgccctgtagcggcgcattaagcgcggcgggtgtggtggttacgcgcagcgtgaccg |
| | | ctacacttgccagcgccctagegcccgctcctttcgctttcttcccttcctttctcgccacgttc |
| | | gccggctttccccgtcaagctctaaatcgggggctccctttagggttccgatttagtgctttac |
| | | ggcacctcgaccccaaaaaacttgattagggtgatggttcacgtagtgggccatcgccctg |
| | | atagacggtttttcgccctttgacgttggagtccacgttctttaatagtggactcttgttccaaac |
| | | tggaacaacactcaaccctatctcggtctattcttttgatttataagggattttgccgatttcggc |
| | | ctattggttaaaaaatgagctgatttaacaaaaatttaacgcgaattaattctgtggaatgtgtg |
| | | tcagttagggtgtggaaagtccccaggctccccagcaggcagaagtatgcaaagcatgca |
| | | tctcaattagtcagcaaccaggtgtggaaagtccccaggctccccagcaggcagaagtat |
| | | gcaaagcatgcatctcaattagtcagcaaccatagtcccgcccctaactccgcccatcccg |
| | | cccctaactccgcccagttccgcccattctccgccccatggctgactaattttttttatttatgca |
| | | gaggccgaggccgcctctgcctctgagctattccagaagtagtgaggaggcttttttggag |
| | | gcctaggcttttgcaaaaagctcccgggagcttgtatatccattttcggatctgatcaagaga |
| | | caggatgaggatcgtttcgcatggtggagtacaagcccacggtgcgcctcgccacccgcg |
| | | acgacgtccccagggccgtacgcaccctcgccgccgegttcgccgactaccccgccacg |
| | | cgccacaccgtcgatccggaccgccacatcgagcgggtcaccgagctgcaagaactctt |
| | | cctcacgcgcgtcgggctcgacatcggcaaggtgtgggtcgcggacgacggcgccgcg |
| | | gtggcggtctggaccacgccggagagcgtcgaagcgggggcggtgttcgccgagatcg |
| | | gcccgcgcatggccgagttgagcggttcccggctggccgcgcagcaacagatggaagg |
| | | cctcctggcgccgcaccggcccaaggagcccgcgtggttcctggccaccgtcggcgtct |
| | | cgcccgaccaccagggcaagggtctgggcagcgccgtcgtgctccccggagtggaggc |
| | | ggccgagcgcgccggggtgcccgccttcctggagacctccgegccccgcaacctcccct |
| | | tctacgagcggctcggcttcaccgtcaccgccgacgtcgaggtgcccgaaggaccgcgc |
| | | acctggtgcatgacccgcaagcccggtgccgcgggactctggggttcgaaatgaccgac |
| | | caagcgacgcccaacctgccatcacgagatttcgattccaccgccgccttctatgaaaggtt |
| | | gggcttcggaatcgttttccgggacgccggctggatgatcctccagcgcggggatctcatg |
| | | ctggagttcttcgcccaccccaacttgtttattgcagcttataatggttacaaataaagcaata |
| | | gcatcacaaatttcacaaataaagcatttttttcactgcattctagttgtggtttgtccaaactcat |
| | | caatgtatcttatcatgtctgtataccgtcgacctctagctagagcttggcgtaatcatggtcat |
| | | agctgtttcctgtgtgaaattgttatccgctcacaattccacacaacatacgagccggaagca |
| | | taaagtgtaaagcctggggtgcctaatgagtgagctaactcacattaattgcgttgcgctcac |
| | | tgcccgctttccagtcgggaaacctgtcgtgccagctgcattaatgaatcggccaacgcgc |
| | | ggggagaggcggtttgcgtattgggcgctcttccgcttcctcgctcactgactcgctgcgct |
| | | cggtcgttcggctgcggcgagcggtatcagctcactcaaaggcggtaatacggttatccac |
| | | agaatcaggggataacgcaggaaagaacatgtgagcaaaaggccagcaaaaggccag |
| | | gaaccgtaaaaaggccgcgttgctggcgtttttccataggctccgcccccctgacgagcat |
| | | cacaaaaatcgacgctcaagtcagaggtggcgaaacccgacaggactataaagatacca |
| | | ggcgtttccccctggaagctccctcgtgcgctctcctgttccgaccctgccgcttaccggat |
| | | acctgtccgcctttctcccttcgggaagcgtggcgctttctcatagctcacgctgtaggtatct |
| | | cagttcggtgtaggtcgttcgctccaagctgggctgtgtgcacgaaccccccgttcagccc |
| | | gaccgctgcgccttatccggtaactatcgtcttgagtccaacccggtaagacacgacttatc |
| | | gccactggcagcagccactggtaacaggattagcagagcgaggtatgtaggcggtgcta |
| | | cagagttcttgaagtggtggcctaactacggctacactagaagaacagtatttggtatctgcg |
| | | ctctgctgaagccagttaccttcggaaaaagagttggtagctcttgatccggcaaacaaacc |
| | | accgctggtagcggtggtttttttgtttgcaagcagcagattacgcgcagaaaaaaaggatc |
| | | tcaagaagatcctttgatcttttctacggggtctgacgctcagtggaacgaaaactcacgtta |
| | | agggattttggtcatgagattatcaaaaaggatcttcacctagatccttttaaattaaaaatgaa |
| | | gttttaaatcaatctaaagtatatatgagtaaacttggtctgacagttaccaatgcttaatcagtg |
| | | aggcacctatctcagcgatctgtctatttcgttcatccatagttgcctgactccccgtcgtgtag |
| | | ataactacgatacgggagggcttaccatctggccccagtgctgcaatgataccgcgagac |
| | | ccacgctcaccggctccagatttatcagcaataaaccagccagccggaagggccgagcg |
| | | cagaagtggtcctgcaactttatccgcctccatccagtctattaattgttgccgggaagctag |
| | | agtaagtagttcgccagttaatagtttgcgcaacgttgttgccattgctacaggcatcgtggtg |
| | | tcacgctcgtcgtttggtatggcttcattcagctccggttcccaacgatcaaggcgagttacat |
| | | gatcccccatgttgtgcaaaaaagcggttagctccttcggtcctccgatcgttgtcagaagta |
| | | agttggccgcagtgttatcactcatggttatggcagcactgcataattctcttactgtcatgcc |
| | | atccgtaagatgcttttctgtgactggtgagtactcaaccaagtcattctgagaatagtgtatg |
| | | cggcgaccgagttgctcttgcccggcgtcaatacgggataataccgcgccacatagcaga |
| | | actttaaaagtgctcatcattggaaaacgttcttcggggcgaaaactctcaaggatcttaccg |
| | | ctgttgagatccagttcgatgtaacccactcgtgcacccaactgatcttcagcatcttttacttt |
| | | caccagcgtttctgggtgagcaaaaacaggaaggcaaaatgccgcaaaaaagggaataa |
| | | gggcgacacggaaatgttgaatactcatactcttcctttttcaatattattgaagcatttatcag |
| | | ggttattgtctcatgagcggatacatatttgaatgtatttagaaaaataaacaaataggggttc |
| | | cgcgcacatttccccgaaaagtgccacctgacgtc |
| p404 | 139 | gacggatcgggagatctcccgatcccctatggtgcactctcagtacaatctgctctgatgcc |
| | | gcatagttaagccagtatctgctccctgcttgtgtgttggaggtcgctgagtagtgcgcgag |
| | | caaaatttaagctacaacaaggcaaggcttgaccgacaattgcatgaagaatctgcttagg |
| | | gttaggcgttttgcgctgcttcgcgatgtacgggccagatatacgcgttgacattgattattga |
| | | ctagttattaatagtaatcaattacggggtcattagttcatagcccatatatggagttccgcgtt |
| | | acataacttacggtaaatggcccgcctggctgaccgcccaacgacccccgcccattgacg |
| | | tcaataatgacgtatgttcccatagtaacgccaatagggactttccattgacgtcaatgggtg |
| | | gagtatttacggtaaactgcccacttggcagtacatcaagtgtatcatatgccaagtacgccc |
| | | cctattgacgtcaatgacggtaaatggcccgcctggcattatgcccagtacatgaccttatg |
| | | ggactttcctacttggcagtacatctacgtattagtcatcgctattaccatggtgatgcggtttt |
| | | ggcagtacatcaatgggcgtggatagcggtttgactcacggggatttccaagtctccaccc |
| | | cattgacgtcaatgggagtttgttttggcaccaaaatcaacgggactttccaaaatgtcgtaa |
| | | caactccgccccattgacgcaaatgggcggtaggcgtgtacggtgggaggtctatataag |
| | | cagagctctctggctaactagagaacccactgcttactggcttatcgaaattaatacgactca |
| | | ctatagggagacccaagctggctagcgccaccatggtgtctaagggcgaggaactgttca |
| | | ccggcgtggtgcctatcctggtggaactggacggagatgtgaacggccataagtttagcgt |
| | | gtccggcgaaggcgaaggcgacgccacctacggcaagctgaccctgaagttcatctgta |
| | | ccaccggcaaactgcccgtgccatggcctaccctggtgacaacactgacctatggcgttca |
| | | gtgcttcagcagataccccgaccacatgaaacagcacgatttctttaaaagcgccatgcctg |
| | | agggctacgtgcaggagTGCacaatcttcttcaaagacgacggaaattacaagaccag |
| | | agccgaggtgaagttcgagggcgatacactggtcaatagaatcgagctgaagggaatcg |
| | | actttaaggaagatggcaacatcctgggccacaagctcgagtacaactacaacagccaca |
| | | acgtgtacatcatggctgataagcagaagaacggcatcaaggtgaatttcaagatccggca |
| | | caacatcgaggacggcagcgtgcaactggccgaccactaccagcagaacacccctattg |
| | | gagatggccctgtgctgctgcctgacaaccactacctgtctacacagagcaaactgtccaa |
| | | ggaccccaacgagaagcgggaccacatggtcctgctggaattcgtgaccgccgctggca |
| | | tcacactgggaatggacgagctgtacaagggcggcagcggcggatctatggtgtccaag |
| | | ggcgaggaagataatatggccatcatcaaggaattcatgcggttcaaagtgcacatggaag |
| | | gcagcgtgaacggccatgagtttgagatcgagggagaaggcgagggtagaccttacgag |
| | | ggcacccagaccgccaagctgaaggtgacaaagggcggccctctgccattcgcctggga |
| | | cattctgagccctcagttcatgtacggctctaaggcttatgtgaagcaccccgctgatatccc |
| | | tgactatctgaagctctccttccccgagggcttcaagtgggaacgggtgatgaacttcgagg |
| | | atggcggagtggtgacagtgacacaagacagcagcctgcaggacggcgaattcatctac |
| | | aaagtgaagctgagaggcaccaattttccatctgatggacctgtgatgcagaaaaagacca |
| | | tgggctgggaggccagcagcgagagaatgtaccctgaggacggagctctgaagggcga |
| | | aatcaaacagagactgaagctgaaggacggaggccactacgacgccgaagtgaaaaca |
| | | acctacaaggccaagaagcccgtccagctgcctggcgcctacaacgttaacatcaagctg |
| | | gacatcaccagccacaacgaggattacaccatcgtggaacagtacgagcgcgccgaagg |
| | | aagacacagcaccggcggcatggacgagctgtacaagtgaaagcttggtaccgagctcg |
| | | gatccactagtccagtgtggtggaattctgcagatatccagcacagtggcggccgctcgag |
| | | tctagagggcccgtttaaacccgctgatcagcctcgactgtgccttctagttgccagccatct |
| | | gttgtttgcccctcccccgtgccttccttgaccctggaaggtgccactcccactgtcctttcct |
| | | aataaaatgaggaaattgcatcgcattgtctgagtaggtgtcattctattctggggggtgggg |
| | | tggggcaggacagcaagggggaggattgggaagacaatagcaggcatgctggggatgc |
| | | ggtgggctctatggcttctgaggcggaaagaaccagctggggctctagggggtatcccca |
| | | cgcgccctgtagcggcgcattaagcgcggcgggtgtggtggttacgcgcagcgtgaccg |
| | | ctacacttgccagcgccctagegcccgctcctttcgctttcttcccttcctttctegccacgttc |
| | | gccggctttccccgtcaagctctaaatcgggggctccctttagggttccgatttagtgctttac |
| | | ggcacctcgaccccaaaaaacttgattagggtgatggttcacgtagtgggccatcgccctg |
| | | atagacggtttttcgccctttgacgttggagtccacgttctttaatagtggactcttgttccaaac |
| | | tggaacaacactcaaccctatctcggtctattcttttgatttataagggattttgccgatttcggc |
| | | ctattggttaaaaaatgagctgatttaacaaaaatttaacgcgaattaattctgtggaatgtgtg |
| | | tcagttagggtgtggaaagtccccaggctccccagcaggcagaagtatgcaaagcatgca |
| | | tctcaattagtcagcaaccaggtgtggaaagtccccaggctccccagcaggcagaagtat |
| | | gcaaagcatgcatctcaattagtcagcaaccatagtcccgcccctaactccgcccatcccg |
| | | cccctaactccgcccagttccgcccattctccgccccatggctgactaattttttttatttatgca |
| | | gaggccgaggccgcctctgcctctgagctattccagaagtagtgaggaggcttttttggag |
| | | gcctaggcttttgcaaaaagctcccgggagcttgtatatccattttcggatctgatcaagaga |
| | | caggatgaggatcgtttcgcatggtggagtacaagcccacggtgcgcctcgccacccgcg |
| | | acgacgtccccagggccgtacgcaccctcgccgccgegttcgccgactaccccgccacg |
| | | cgccacaccgtcgatccggaccgccacatcgagcgggtcaccgagctgcaagaactctt |
| | | cctcacgcgcgtcgggctcgacatcggcaaggtgtgggtcgcggacgacggcgccgcg |
| | | gtggcggtctggaccacgccggagagcgtcgaagcgggggcggtgttcgccgagatcg |
| | | gcccgcgcatggccgagttgagcggttcccggctggccgcgcagcaacagatggaagg |
| | | cctcctggcgccgcaccggcccaaggagcccgcgtggttcctggccaccgtcggcgtct |
| | | cgcccgaccaccagggcaagggtctgggcagcgccgtcgtgctccccggagtggaggc |
| | | ggccgagcgcgccggggtgcccgccttcctggagacctccgegccccgcaacctcccct |
| | | tctacgagcggctcggcttcaccgtcaccgccgacgtcgaggtgcccgaaggaccgcgc |
| | | acctggtgcatgacccgcaagcccggtgccgcgggactctggggttcgaaatgaccgac |
| | | caagcgacgcccaacctgccatcacgagatttcgattccaccgccgccttctatgaaaggtt |
| | | gggcttcggaatcgttttccgggacgccggctggatgatcctccagcgcggggatctcatg |
| | | ctggagttcttcgcccaccccaacttgtttattgcagcttataatggttacaaataaagcaata |
| | | gcatcacaaatttcacaaataaagcatttttttcactgcattctagttgtggtttgtccaaactcat |
| | | caatgtatcttatcatgtctgtataccgtcgacctctagctagagcttggcgtaatcatggtcat |
| | | agctgtttcctgtgtgaaattgttatccgctcacaattccacacaacatacgagccggaagca |
| | | taaagtgtaaagcctggggtgcctaatgagtgagctaactcacattaattgcgttgcgctcac |
| | | tgcccgctttccagtcgggaaacctgtcgtgccagctgcattaatgaatcggccaacgcgc |
| | | ggggagaggcggtttgcgtattgggcgctcttccCATCGGTGCAGTTTCGA |
| | | GGGTAAAGCCTTTGGCGCGGTGATGTGGACTTTTGTTC |
| | | TCTAACTACAACTCCCAGCATACGTCACCCCTCACGTG |
| | | GGCGCTAGGTGTGGTTTCGTGGGATAGGGTAAGGCGA |
| | | AAATGAAGTGACGATGACGTACCAAAGAGGAAAATAG |
| | | TTGAATATGTTGTGTGAGCGCGTCGTTTGCAGAAGCCC |
| | | CAGTGGCCTAATGGATAAGGCATTGGCCTGCAAAGCC |
| | | AGGGATTGTGGGTTCGAGTCCCATCTGGGGTGGCCTGT |
| | | GACTTTtgtccttttttcccctttctaaaCAAAGCGAAATATgccaactttact |
| | | ttttttttttttaaatgggaagtgtgtgtgtgtttttactaGCTGGTGGGCTGCAAT |
| | | TTTCGAGAATCTGCACACACCCATGTCCTTTGTCTGAG |
| | | CCGCAGGTCCCCTGGGATTTGGGGAGACGTCAATACGT |
| | | TGAATTTgcttcctcgctcactgactcgctgcgctcggtcgttcggctgcggcgagc |
| | | ggtatcagctcactcaaaggcggtaatacggttatccacagaatcaggggataacgcagg |
| | | aaagaacatgtgagcaaaaggccagcaaaaggccaggaaccgtaaaaaggccgcgttg |
| | | ctggcgtttttccataggctccgcccccctgacgagcatcacaaaaatcgacgctcaagtca |
| | | gaggtggcgaaacccgacaggactataaagataccaggcgtttccccctggaagctccct |
| | | cgtgcgctctcctgttccgaccctgccgcttaccggatacctgtccgcctttctcccttcggg |
| | | aagcgtggcgctttctcatagctcacgctgtaggtatctcagttcggtgtaggtcgttcgctcc |
| | | aagctgggctgtgtgcacgaaccccccgttcagcccgaccgctgcgccttatccggtaact |
| | | atcgtcttgagtccaacccggtaagacacgacttatcgccactggcagcagccactggtaa |
| | | caggattagcagagcgaggtatgtaggcggtgctacagagttcttgaagtggtggcctaac |
| | | tacggctacactagaagaacagtatttggtatctgcgctctgctgaagccagttaccttcgga |
| | | aaaagagttggtagctcttgatccggcaaacaaaccaccgctggtagcggtggtttttttgttt |
| | | gcaagcagcagattacgcgcagaaaaaaaggatctcaagaagatcctttgatcttttctacg |
| | | gggtctgacgctcagtggaacgaaaactcacgttaagggattttggtcatgagattatcaaa |
| | | aaggatcttcacctagatccttttaaattaaaaatgaagttttaaatcaatctaaagtatatatga |
| | | gtaaacttggtctgacagttaccaatgcttaatcagtgaggcacctatctcagcgatctgtcta |
| | | tttcgttcatccatagttgcctgactccccgtcgtgtagataactacgatacgggagggcttac |
| | | catctggccccagtgctgcaatgataccgcgagacccacgctcaccggctccagatttatc |
| | | agcaataaaccagccagccggaagggccgagcgcagaagtggtcctgcaactttatccg |
| | | cctccatccagtctattaattgttgccgggaagctagagtaagtagttcgccagttaatagttt |
| | | gcgcaacgttgttgccattgctacaggcatcgtggtgtcacgctcgtcgtttggtatggcttc |
| | | attcagctccggttcccaacgatcaaggcgagttacatgatcccccatgttgtgcaaaaaag |
| | | cggttagctccttcggtcctccgatcgttgtcagaagtaagttggccgcagtgttatcactcat |
| | | ggttatggcagcactgcataattctcttactgtcatgccatccgtaagatgcttttctgtgactg |
| | | gtgagtactcaaccaagtcattctgagaatagtgtatgcggcgaccgagttgctcttgcccg |
| | | gcgtcaatacgggataataccgcgccacatagcagaactttaaaagtgctcatcattggaaa |
| | | acgttcttcggggcgaaaactctcaaggatcttaccgctgttgagatccagttcgatgtaacc |
| | | cactcgtgcacccaactgatcttcagcatcttttactttcaccagcgtttctgggtgagcaaaa |
| | | acaggaaggcaaaatgccgcaaaaaagggaataagggcgacacggaaatgttgaatact |
| | | catactcttcctttttcaatattattgaagcatttatcagggttattgtctcatgagcggatacatat |
| | | ttgaatgtatttagaaaaataaacaaataggggttccgcgcacatttccccgaaaagtgcca |
| | | cctgacgtc |
| Expression | 140 | GGAGAATAGGAAGGCTGCCGCTCCAATGGGGCTGCTG |
| cassette for | | ACGTCTGCTGGAGGGAAGGCATGGTCTGCGCCACCGA |
| Ser- | | GCAGAGGCGCACGAGCGCAGACGTCTCTAGGCGGCGC |
| tRNA^{Arg(CCG)} | | GGGTGGAGCCGGGCGGAGCCGGGCGGAGCCGGCTGGA |
| | | GGCGGGCGTGTTGATAGGCAGCTATAGTTGCACAGGG |
| | | CCGACAGCGCACGCGGCTGTGATGGCCGAGTGGTTA |
| | | AGGCGTTGGACTCCGAATCCAATGGGGTCTCCCCG |
| | | CGCAGGTTCGAATCCTGCTCACAGCGTCACTAGTTTT |
| | | GGTATTTAGGTATCTCGGTCTCTGGAGAAGGAAAAAGC |
| | | AGTTTGTTTTCTGGTTCTTTCCTTGGAGAATCTCCCGCG |
| | | TTATTCTGAGAATCTCAGAGACTCGGCCGTCAGGGACC |
| | | AGCCCGCGAGCGCCCTCTTGCGATTCTCATAGTCCTAT |
| | | CGACCTGCAAAAACTTTTTTACTGCGGATTTCAATT |
| Expression | 141 | ATCGGTGCAGTTTCGAGGGTAAAGCCTTTGGCGCGGTG |
| cassette for | | ATGTGGACTTTTGTTCTCTAACTACAACTCCCAGCATA |
| Arg- | | CGTCACCCCTCACGTGGGCGCTAGGTGTGGTTTCGTGG |
| tRNA^{Cys(GCA)} | | GATAGGGTAAGGCGAAAATGAAGTGACGATGACGTAC |
| | | CAAAGAGGAAAATAGTTGAATATGTTGTGTGAGCGCG |
| | | TCGTTTGCAGAAGCCCCAGTGGCCTAATGGATAAGG |
| | | CATTGGCCTGCAAAGCCAGGGATTGTGGGTTCGAG |
| | | TCCCATCTGGGGTGGCCTGTGACTTTTGTCCTTTTTTC |
| | | CCCTTTCTAAACAAAGCGAAATATGCCAACTTTACTTT |
| | | TTTTTTTTTTAAATGGGAAGTGTGTGTGTGTTTTTACTA |
| | | GCTGGTGGGCTGCAATTTTCGAGAATCTGCACACACCC |
| | | ATGTCCTTTGTCTGAGCCGCAGGTCCCCTGGGATTTGG |
| | | GGAGACGTCAATACGTTGAATTT |
| Expression | 142 | ATCGGTGCAGTTTCGAGGGTAAAGCCTTTGGCGCGGTG |
| cassette for | | ATGTGGACTTTTGTTCTCTAACTACAACTCCCAGCATA |
| Arg- | | CGTCACCCCTCACGTGGGCGCTAGGTGTGGTTTCGTGG |
| tRNA^{His(GUG)} | | GATAGGGTAAGGCGAAAATGAAGTGACGATGACGTAC |
| | | CAAAGAGGAAAATAGTTGAATATGTTGTGTGAGCGCG |
| | | TCGTTTGCAGAAGCCCCAGTGGCCTAATGGATAAGG |
| | | CATTGGCCTGTGAAGCCAGGGATTGTGGGTTCGAG |
| | | TCCCATCTGGGGTGGCCTGTGACTTTTGTCCTTTTTTC |
| | | CCCTTTCTAAACAAAGCGAAATATGCCAACTTTACTTT |
| | | TTTTTTTTTTAAATGGGAAGTGTGTGTGTGTTTTTACTA |
| | | GCTGGTGGGCTGCAATTTTCGAGAATCTGCACACACCC |
| | | ATGTCCTTTGTCTGAGCCGCAGGTCCCCTGGGATTTGG |
| | | GGAGACGTCAATACGTTGAATTT |
| Expression | 143 | ATCGGTGCAGTTTCGAGGGTAAAGCCTTTGGCGCGGTG |
| cassette for | | ATGTGGACTTTTGTTCTCTAACTACAACTCCCAGCATA |
| Arg- | | CGTCACCCCTCACGTGGGCGCTAGGTGTGGTTTCGTGG |
| tRNA^{Gln(CUG)} | | GATAGGGTAAGGCGAAAATGAAGTGACGATGACGTAC |
| | | CAAAGAGGAAAATAGTTGAATATGTTGTGTGAGCGCG |
| | | TCGTTTGCAGAAGCCCCAGTGGCCTAATGGATAAGG |
| | | CATTGGCCTCTGAAGCCAGGGATTGTGGGTTCGAG |
| | | TCCCATCTGGGGTGGCCTGTGACTTTTGTCCTTTTTTC |
| | | CCCTTTCTAAACAAAGCGAAATATGCCAACTTTACTTT |
| | | TTTTTTTTTTAAATGGGAAGTGTGTGTGTGTTTTTACTA |
| | | GCTGGTGGGCTGCAATTTTCGAGAATCTGCACACACCC |
| | | ATGTCCTTTGTCTGAGCCGCAGGTCCCCTGGGATTTGG |
| | | GGAGACGTCAATACGTTGAATTT |
| Expression | 144 | ATCGGTGCAGTTTCGAGGGTAAAGCCTTTGGCGCGGTG |
| cassette for | | ATGTGGACTTTTGTTCTCTAACTACAACTCCCAGCATA |
| Arg- | | CGTCACCCCTCACGTGGGCGCTAGGTGTGGTTTCGTGG |
| tRNA^{Trp(CCA)} | | GATAGGGTAAGGCGAAAATGAAGTGACGATGACGTAC |
| | | CAAAGAGGAAAATAGTTGAATATGTTGTGTGAGCGCG |
| | | TCGTTTGCAGAAGCCCCAGTGGCCTAATGGATAAGG |
| | | CATTGGCCTCCAAAGCCAGGGATTGTGGGTTCGAG |
| | | TCCCATCTGGGGTGGCCTGTGACTTTTGTCCTTTTTTC |
| | | CCCTTTCTAAACAAAGCGAAATATGCCAACTTTACTTT |
| | | TTTTTTTTTTAAATGGGAAGTGTGTGTGTGTTTTTACTA |
| | | GCTGGTGGGCTGCAATTTTCGAGAATCTGCACACACCC |
| | | ATGTCCTTTGTCTGAGCCGCAGGTCCCCTGGGATTTGG |
| | | GGAGACGTCAATACGTTGAATTT |

TABLE 2
Single nucleotide variations in genetic diseases
characterized by a missense mutation

| SNV | SNV type | Gene |
|---|---|---|
| NM_000033.4(ABCD1):c.1850G > A | Arg-His G > A | ABCD1 |
| NM_000033.4(ABCD1):c.1166G > A | Arg-His G > A | ABCD1 |
| NM_000033.4(ABCD1):c.1661G > A | Arg-His G > A | ABCD1 |
| NM_000033.4(ABCD1):c.311G > A | Arg-His G > A | ABCD1 |
| NM_000033.4(ABCD1):c.1849C > T | Arg-Cys C > T | ABCD1 |
| NM_000033.4(ABCD1):c.838C > T | Arg-Cys C > T | ABCD1 |
| NM_000033.4(ABCD1):c.454C > T | Arg-Cys C > T | ABCD1 |
| NM_000033.4(ABCD1):c.1252C > T | Arg-Trp C > T | ABCD1 |
| NM_000033.4(ABCD1):c.1552C > T | Arg-Trp C > T | ABCD1 |
| NM_000033.4(ABCD1):c.1771C > T | Arg-Trp C > T | ABCD1 |
| NM_000033.4(ABCD1):c.565C > T | Arg-Trp C > T | ABCD1 |
| NM_000033.4(ABCD1):c.1978C > T | Arg-Trp C > T | ABCD1 |
| NM_000033.4(ABCD1):c.1202G > A | Arg-Gln G > A | ABCD1 |
| NM_000033.4(ABCD1):c.1553G > A | Arg-Gln G > A | ABCD1 |
| NM_000033.4(ABCD1):c.1772G > A | Arg-Gln G > A | ABCD1 |
| NM_000033.4(ABCD1):c.1979G > A | Arg-Gln G > A | ABCD1 |
| NM_000169.3(GLA):c.335G > A | Arg-His G > A | GLA |
| NM_000169.3(GLA):c.1088G > A | Arg-His G > A | GLA |
| NM_000169.3(GLA):c.334C > T | Arg-Cys C > T | GLA |
| NM_000169.3(GLA):c.1087C > T | Arg-Cys C > T | GLA |
| NM_000169.3(GLA):c.1066C > T | Arg-Trp C > T | GLA |
| NM_000169.3(GLA):c.902G > A | Arg-Gln G > A | GLA |
| NM_000169.3(GLA):c.680G > A | Arg-Gln G > A | GLA |
| NM_000169.3(GLA):c.1025G > A | Arg-Gln G > A | GLA |
| NM_000157.4(GBA):c.1604G > A | Arg-His G > A | GBA |
| NM_000157.4(GBA):c.1504C > T | Arg-Cys C > T | GBA |
| NM_000157.4(GBA):c.508C > T | Arg-Cys C > T | GBA |
| NM_000157.4(GBA):c.625C > T | Arg-Cys C > T | GBA |
| NM_000157.4(GBA):c.1603C > T | Arg-Cys C > T | GBA |
| NM_000157.4(GBA):c.259C > T | Arg-Trp C > T | GBA |
| NM_000157.4(GBA):c.475C > T | Arg-Trp C > T | GBA |
| NM_000153.4(GALC):c.1592G > A | Arg-His G > A | GALC |
| NM_000153.4(GALC):c.1591C > T | Arg-Cys C > T | GALC |
| NM_000153.4(GALC):c.868C > T | Arg-Cys C > T | GALC |
| NM_000153.4(GALC):c.1186C > T | Arg-Trp C > T | GALC |
| NM_000487.6(ARSA):c.869G > A | Arg-His G > A | ARSA |
| NM_000487.6(ARSA):c.737G > A | Arg-His G > A | ARSA |
| NM_000487.6(ARSA):c.736C > T | Arg-Cys C > T | ARSA |
| NM_000487.6(ARSA):c.868C > T | Arg-Cys C > T | ARSA |
| NM_000487.6(ARSA):c.1114C > T | Arg-Trp C > T | ARSA |
| NM_000487.6(ARSA):c.257G > A | Arg-Gln G > A | ARSA |
| NM_000487.6(ARSA):c.938G > A | Arg-Gln G > A | ARSA |
| NM_000199.5(SGSH):c.734G > A | Arg-His G > A | SGSH |
| NM_000199.5(SGSH):c.221G > A | Arg-His G > A | SGSH |
| NM_152419.3(HGSNAT):c.1030C > T | Arg-Cys C > T | HGSNAT |
| NM_000199.5(SGSH):c.220C > T | Arg-Cys C > T | SGSH |
| NM_000199.5(SGSH):c.544C > T | Arg-Cys C > T | SGSH |
| NM_000199.5(SGSH):c.1129C > T | Arg-Cys C > T | SGSH |
| NM_000199.5(SGSH):c.1297C > T | Arg-Trp C > T | SGSH |
| NM_000199.5(SGSH):c.1298G > A | Arg-Gln G > A | SGSH |
| NM_000199.5(SGSH):c.449G > A | Arg-Gln G > A | SGSH |
| NM_000202.8(IDS):c.263G > A | Arg-His G > A | IDS |
| NM_000202.8(IDS):c.262C > T | Arg-Cys C > T | IDS |
| NM_000202.8(IDS):c.1402C > T | Arg-Trp C > T | IDS |
| NM_000531.6(OTC):c.119G > A | Arg-His G > A | OTC |
| NM_000531.6(OTC):c.386G > A | Arg-His G > A | OTC |
| NM_000531.6(OTC):c.118C > T | Arg-Cys C > T | OTC |
| NM_000531.6(OTC):c.829C > T | Arg-Trp C > T | OTC |
| NM_000531.6(OTC):c.422G > A | Arg-Gln G > A | OTC |
| NM_000531.6(OTC):c.77G > A | Arg-Gln G > A | OTC |
| NM_000531.6(OTC):c.275G > A | Arg-Gln G > A | OTC |
| NM_000531.6(OTC):c.830G > A | Arg-Gln G > A | OTC |
| NM_001360.3(DHCR7):c.725G > A | Arg-His G > A | DHCR7 |
| NM_001360.3(DHCR7):c.1210C > T | Arg-Cys C > T | DHCR7 |
| NM_001360.3(DHCR7):c.724C > T | Arg-Cys C > T | DHCR7 |
| NM_001360.3(DHCR7):c.1054C > T | Arg-Trp C > T | DHCR7 |
| NM_001360.3(DHCR7):c.1055G > A | Arg-Gln G > A | DHCR7 |
| NM_001360.3(DHCR7):c.1337G > A | Arg-Gln G > A | DHCR7 |
| NM_000520.6(HEXA):c.1496G > A | Arg-His G > A | HEXA |
| NM_000520.6(HEXA):c.1511G > A | Arg-His G > A | HEXA |
| NM_000520.6(HEXA):c.1510C > T | Arg-Cys C > T | HEXA |
| NM_000520.6(HEXA):c.1495C > T | Arg-Cys C > T | HEXA |
| NM_000520.6(HEXA):c.508C > T | Arg-Trp C > T | HEXA |
| NM_000520.6(HEXA):c.509G > A | Arg-Gln G > A | HEXA |
| NM_000543.5(SMPD1):c.1493G > A | Arg-His G > A | SMPD1 |
| NM_000543.5(SMPD1):c.1805G > A | Arg-His G > A | SMPD1 |
| NM_000543.5(SMPD1):c.1492C > T | Arg-Cys C > T | SMPD1 |
| NM_000543.5(SMPD1):c.688C > T | Arg-Cys C > T | SMPD1 |
| NM_000543.5(SMPD1):c.1426C > T | Arg-Trp C > T | SMPD1 |
| NM_000543.5(SMPD1):c.1427G > A | Arg-Gln G > A | SMPD1 |
| NM_000018.4(ACADVL):c.1097G > A | Arg-His G > A | ACADVL |
| NM_000018.4(ACADVL):c.1349G > A | Arg-His G > A | ACADVL |
| NM_000018.4(ACADVL):c.1837C > T | Arg-Trp C > T | ACADVL |
| NM_000018.4(ACADVL):c.1405C > T | Arg-Trp C > T | ACADVL |
| NM_000018.4(ACADVL):c.1406G > A | Arg-Gln G > A | ACADVL |
| NM_000018.4(ACADVL):c.1376G > A | Arg-Gln G > A | ACADVL |
| NM_000018.4(ACADVL):c.1358G > A | Arg-Gln G > A | ACADVL |

TABLE 3
Single nucleotide variations in genetic diseases characterized
by a missense mutations or nonsense mutations.

| SNV | SNV type | Gene |
|---|---|---|
| NM_000070.3(CAPN3):c.2306G > A | Arg-Gln G > A | CAPN3 |
| NM_000070.3(CAPN3):c.1469G > A | Arg-Gln G > A | CAPN3 |
| NM_000070.3(CAPN3):c.1343G > A | Arg-His G > A | CAPN3 |
| NM_000070.3(CAPN3):c.1465C > T | Arg-Trp C > T | CAPN3 |
| NM_000070.3(CAPN3):c.1714C > T | Arg-Trp C > T | CAPN3 |
| NM_000070.3(CAPN3):c.1342C > T | Arg-Cys C > T | CAPN3 |
| NM_000070.3(CAPN3):c.1063C > T | Arg-Trp C > T | CAPN3 |
| NM_000070.3(CAPN3):c.865C > T | Arg-Trp C > T | CAPN3 |
| NM_000070.3(CAPN3):c.1466G > A | Arg-Gln G > A | CAPN3 |
| NM_000070.3(CAPN3):c.2242C > G | Arg-Gly C > G | CAPN3 |
| NM_000070.3(CAPN3):c.328C > T | Arg-Stop C > T | CAPN3 |
| NM_000070.3(CAPN3):c.2242C > T | Arg-Stop C > T | CAPN3 |
| NM_000070.3(CAPN3):c.973A > T | Arg-Stop A > T | CAPN3 |
| NM_000070.3(CAPN3):c.772A > T | Arg-Stop A > T | CAPN3 |
| NM_000070.3(CAPN3):c.2380A > T | Arg-Stop A > T | CAPN3 |
| NM_000070.3(CAPN3):c.257C > T | Ser-Phe C > T | CAPN3 |
| NM_000070.3(CAPN3):c.643T > C | Ser-Pro T > C | CAPN3 |

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Results

### Distribution Analysis of Pathogenic Missense Mutations

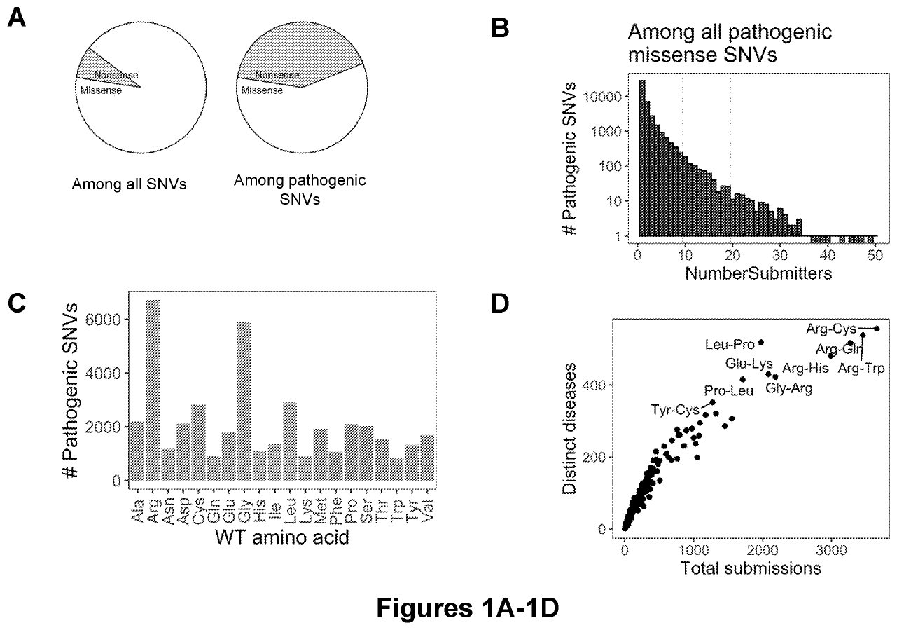

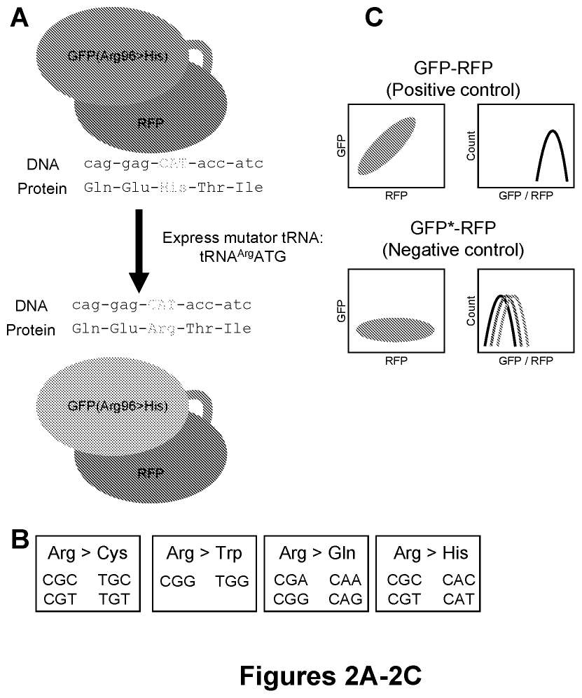

The inventors searched for the reported single nucleotide variants (SNVs) in the ClinVar database (ref.) to obtain detailed information on the type of disease-causing SNVs in CDS. Missense mutations represent 92% of all analyzed CDS SNVs with the rest represented by nonsense mutations among all SNVs; however, many missense mutations are not associated with disease. Among CDS SNVs annotate as “pathogenic”, “likely pathogenic” and “pathogenic/likely pathogenic”, missense mutations represent 58% (FIG. 1A). Among the ~42500 total missense mutations, most have only a single submitter suggesting rare allele frequency, and the vast majority are reported less than 10-times; however, ~100 specific pathogenic SNVs have been reported more than 20-times (FIG. 1B). This result indicates that pathogenic mutations are unevenly distributed in the human genome, and specific hot spot mutations are present that recur at abnormally high frequency.

Separating the pathogenic missense mutations to individual amino acid shows that mutation of Arg to another amino acid represents the highest fraction at >6700 (16% among all pathogenic missense SNVs; 9% among pathogenic SNVs including nonsense mutations), followed by mutation of Gly to another amino acid (FIG. 1C). Pathogenic mutations of the other 18 amino acids are of similar frequency, and are more than 2-fold less than Arg or Gly mutations. Furthermore, merging the type of pathogenic mutations with the reported number of disease occurrence (FIG. 1B) shows that the top 4 frequent pathogenic SNVs are indeed represented by 4 Arg codon mutations to Cys, Trp, Gln, and His (FIG. 1D). These results indicate that Arg mutations to just 4 amino acids represent the highest group of pathogenic single base mutations.

To identify specific diseases that can be treated with MC-tRNA^{Arg} for the Arg codon mutations to Cys, Trp, Gln, and His, the inventors searched ClinVar for diseases with the following criteria:
(i) The disease is genetically recessive.
(ii) The mutation resides in the coding sequence.
(iii) The mutation is associated with a known orphan metabolic disorder.
(iv) Each SNV was submitted by 4 or more submitters.

Applying these criteria, the inventors identified 12 major diseases that are associated with at least 3 of the 4 Arg codon mutations (Table 1). Combining estimates for disease incidence and SNV frequency, the inventors estimate ~55 new patients in the US could be treated per year, using MC-tRNA^{Arg}(GTG) alone. Similarly, MC-tRNA^{Arg}(GCA), MC-tRNA^{Arg}(CCA), and MC-tRNA^{Arg}(TTG) could treat ~49, ~44, and ~51 new patients per year, respectively. All together, the 4 MC-tRNA^{Arg} constructs could treat ~200 new patients in the US per year for this select group of orphan diseases.

Using MC-tRNA^{Arg} to Generate Functional Proteins from DNA Mutations

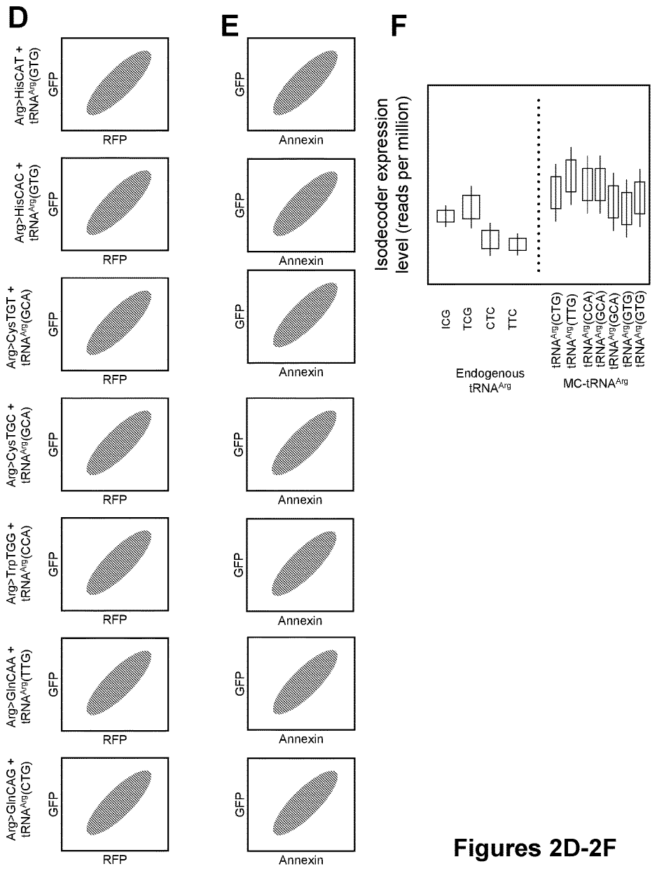

The inventors designed a reporter protein to test the feasibility of using MC-tRNA^{Arg} to generate functional proteins during translation from genes containing Arg mutations at the DNA level that would produce a functionally defective protein in the absence of MC-tRNA^{Arg} (ref.). This dual fluorescent protein reporter contains both green fluorescent protein (GFP) and red fluorescent protein (RFP) in a single polypeptide (FIG. 2A). The Arg96 residue in the GFP is mutated to one of the 7 codons that are present in the most frequent pathogenic Arg mutations (making GFP*) which include Arg to Cys (CGC/CGT-to-TGC/TGT), Trp (CGG-to-TGG), Gln (CGA/CGG-to-CAA/CAG), and His (CGC/CGT-to-CAC/CAT) (FIG. 2B, see below). This mutation increases the maturity time of GFP from hours to months (PMID: 14523232, 16331981, 18470931.), so that GFP is practically non-fluorescent at the laboratory experimental time scale.

| | Arg-to-Cys | Arg-to-Trp | Arg-to-Gln | Arg-to-His |
|---|---|---|---|---|
| Mutant | TGC | TGG | CAA | CAC |
| sequence at | TGT | | CAG | CAT |
| Arg96 of GFP | | | | |

MC-tRNA^{Arg} design is more complex, since many tRNA bodies may fulfill this role. The inventors choose to test the 20 different tRNA^{Arg} sequences in the reference human genome (genomic tRNA database, http://gtmadb.ucsc.edu/). The design changes the anticodon sequence of each of the tRNA^{Arg} to that of the anticodon sequence of human tRNAs that read Cys/Trp/Gln/His sequences (Table 2). For example, human tRNA^{Cys} has a single anticodon sequence of GCA which reads both TGC and TGT codons through G-T wobble. Therefore, MC-tRNA^{Arg}(GCA) is expected to read both TGC and TGT mutants in the GFP Arg96-to-Cys constructs. The natural tRNA^{Arg}(TCT) sequences are represented by 6 tRNA^{Arg}(TCT) genes that contain introns which may influence the decoding efficiency of the MC-tRNA^{Arg}. The inventors therefore also include 6 more constructs containing introns, increasing the total number of MC-tRNA^{Arg} to 26 for each anticodon. Following the MC-tRNA^{Arg}(GCA) example, there is a single native anticodon sequence for Trp (CCA) and His (GTG), so that the design of MC-tRNA^{Arg}(CCA) and MC-tRNA^{Arg}(GTG) has the same consideration as MC-tRNA^{Arg}(GCA). There are two native anticodon sequences for Gln (TTG, CTG); TTG can read both Gln codons of CAA and CAG, whereas CTG can only read CAG. The inventors included both anticodons in the design which makes for MC-tRNA^{Arg}(TTG) and MC-tRNA^{Arg}(CTG). Each backbone is paired with its endogenous promoter and terminator by taking 200 bp upstream and downstream genomic context for plasmid-based expression.

The experimental test includes the following steps. First the GFP*-RFP and one single MC-tRNA^{Arg} present in the same plasmid is transfected into the human cell culture of HEK293T. The inventors also include one positive control of a wild type GFP-RFP construct, and two negative controls, one of just GFP*-RFP construct, and the other of GFP*-RFP plus tRNA^{Arg}(TCG) which only reads Arg codons. After 24, 48 and 72 hours, both green and red fluorescent levels in transfected cells are measured by flow cytometry. The positive control shows high fluorescence for both GFP and RFP along the diagonal (FIG. 2C). All negative controls of GFP*-RFP (FIG. 2C, bottom). The presence of MC-tRNA^{Arg} generates substantial amounts of functional GFP proteins, the actual magnitude of green fluorescence is dependent on both the type of MC-tRNA^{Arg} used and the GFP* mutation in the DNA (FIG. 2D). These results indicate that the MC-tRNA^{Arg} strategy works in human cells to generate functional proteins at the translation level from genetic mutations at the DNA level.

MC-tRNA^{Arg} can in principle reads many non-Arg codons, causing substantial mistranslation. However, mistranslation is not inherently lethal, in fact, cells can tolerate a high level of mistranslation, and even naturally regulate the mistranslation levels and types (ref.). Nevertheless, the inventors performed additional experiments to examine the potential toxic effects of expressing MC-tRNA^{Arg} (FIG. 2E). Both annexin V stain indicate cell death. The inventors observe similar cell death levels in MC-tRNA expression cells and control indicating minimal toxic effects. The inventors found that although some MC-tRNA^{Arg} exhibit strong toxic effects leading to substantial cell death, other MC-tRNA^{Arg} show milder toxicity, indicating that specific MC-tRNA^{Arg} may be obtained to minimize mistranslation-derived toxicity.

To demonstrate that MC-tRNA^{Arg} is indeed expressed in cells, the inventors performed high throughput tRNA sequencing of several transfected cells that showed the highest level of GFP fluorescence (FIG. 2F). MC-tRNA^{Arg} can be distinguished from the endogenous tRNAs by their anticodon sequences. The endogenous tRNA^{Arg}s have anticodons of ICG (GCG in sequencing), TCG, CTC, and TTC, whereas MC-tRNA's have anticodons of GCA, CCA, GTG, TTG, and CTG. MC-tRNA^{Arg} is expressed at appreciable levels, comprising of X-Y % of endogenous tRNA^{Arg} in the experiments.

### MC-tRNA^{Arg} Restores Arg Residues in Disease Associated Peptides

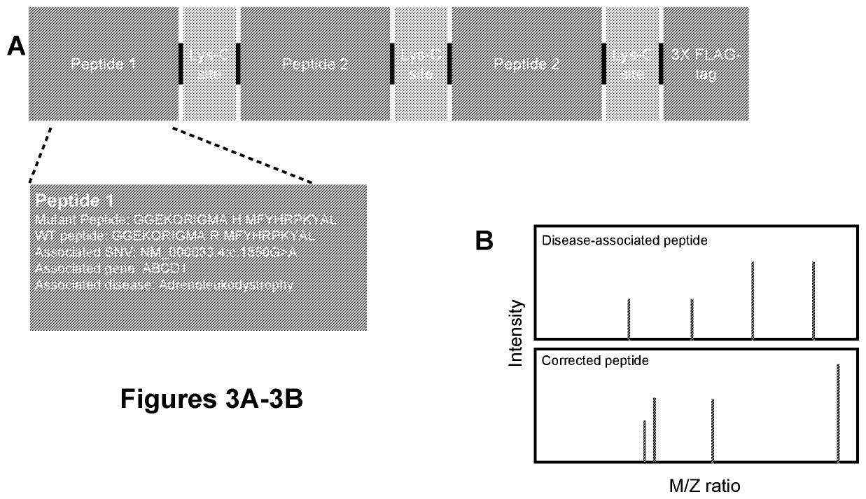

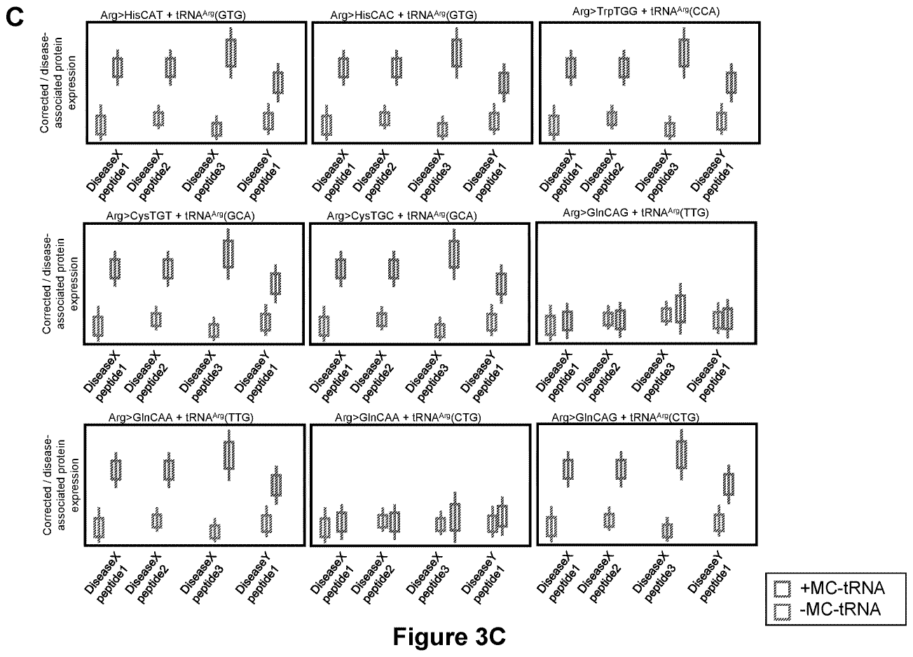

To demonstrate that MC-tRNA^{Arg} indeed reads a non-Arg codon and incorporates Arg in translation, the inventors performed mass spectrometry of reporter protein constructs that include natural peptide sequences in the disease context (FIG. 3A, Table 3). the single polypeptide reporter construct contains fused blocks of 21 amino acid peptide sequence centered at the pathogenic mutation (disease associated peptide), each block separated by a Gly-Lys-Gly sequence which provides a Lys-C protease cleavage site. Each reporter also contains a triple Flag tag near the C-terminus to facilitate purification by immunoprecipitation. The inventors designed a total of 4 constructs, each comprised of a single type of Arg mutations to the same amino acid. For example, all Arg-to-His disease causing mutations are present in the same reporter construct, so that all missense correcting event from His to Arg can be tested with MC-tRNA^{Arg}(GTG). Similar strategies apply for Arg-to-Cys, Arg-to-Trp, Arg-to-Gln reporter constructs to be tested with MC-tRNA^{Arg}(GCA), MC-tRNA^{Arg}(CCA), MC-tRNA^{Arg}(TTG or CTG), respectively.

Our mass spec results show that cells naturally produce the disease-associated peptides in the absence of MC-tRNA^{Arg}. In the presence of the corresponding MC-tRNA^{Arg}, the disease-associated peptides are still produced using the endogenous tRNAs, at the same time, missense correcting peptides are also detected that correspond to X-to-Arg correction during translation (FIG. 3B). This result can be semi-quantitatively compared for each X-to-Arg corrected peptide to disease-associated peptide (FIG. 3C). As expected, in most cases MC-tRNA^{Arg} show appreciable level of producing the correct peptide. The non-correction effect of MC-tRNA^{Arg}(CTG) with Arg-to-Gln(CAA) mutation is expected as MC-tRNA^{Arg}(CTG) can only read the CAG, not the CAA codon of Gln.

Missense Correction Also Occurs with MC-tRNA^{Ser}

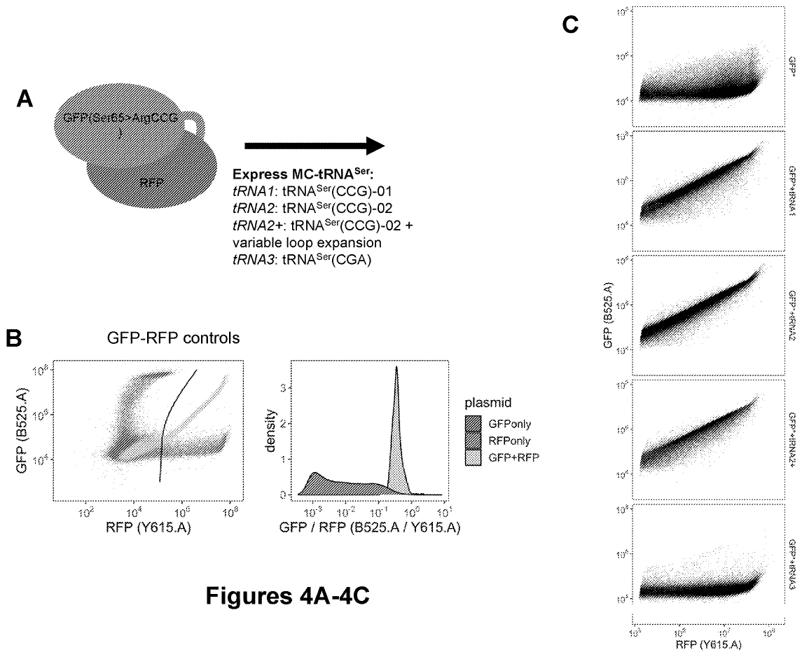

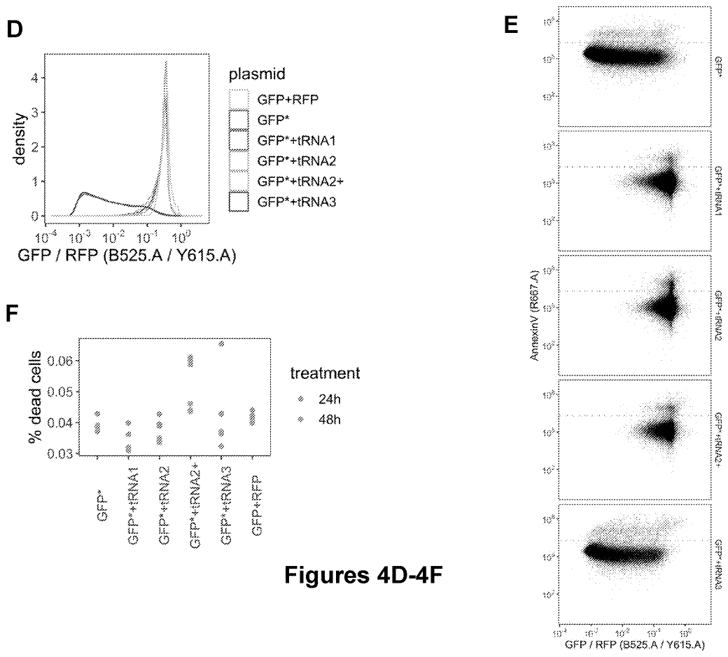

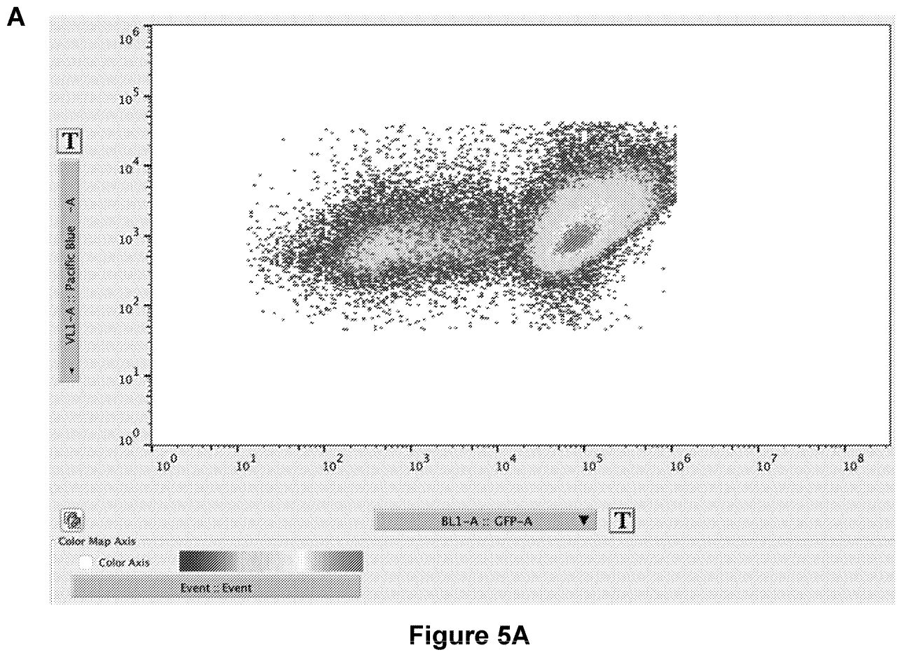

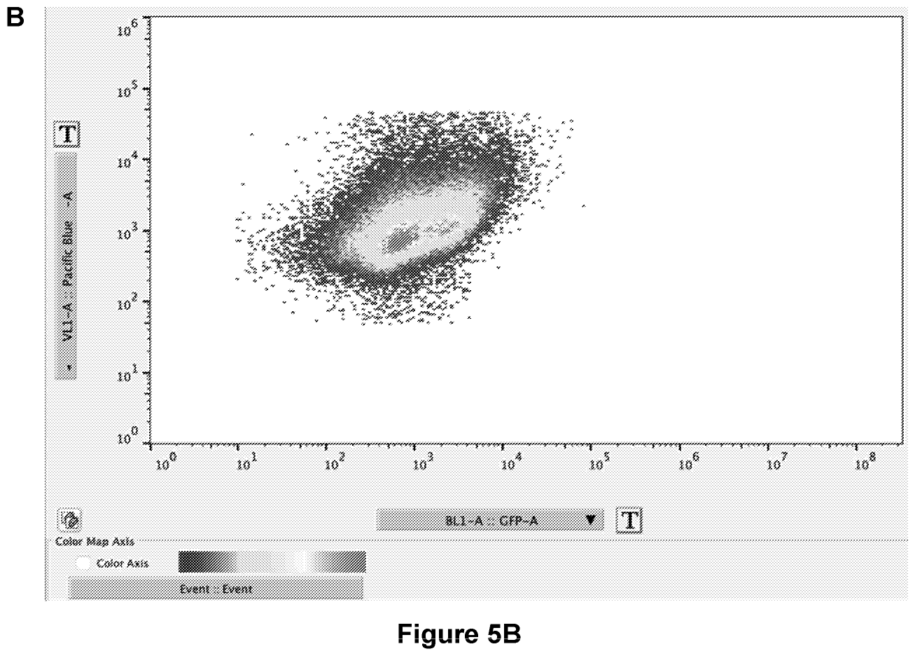

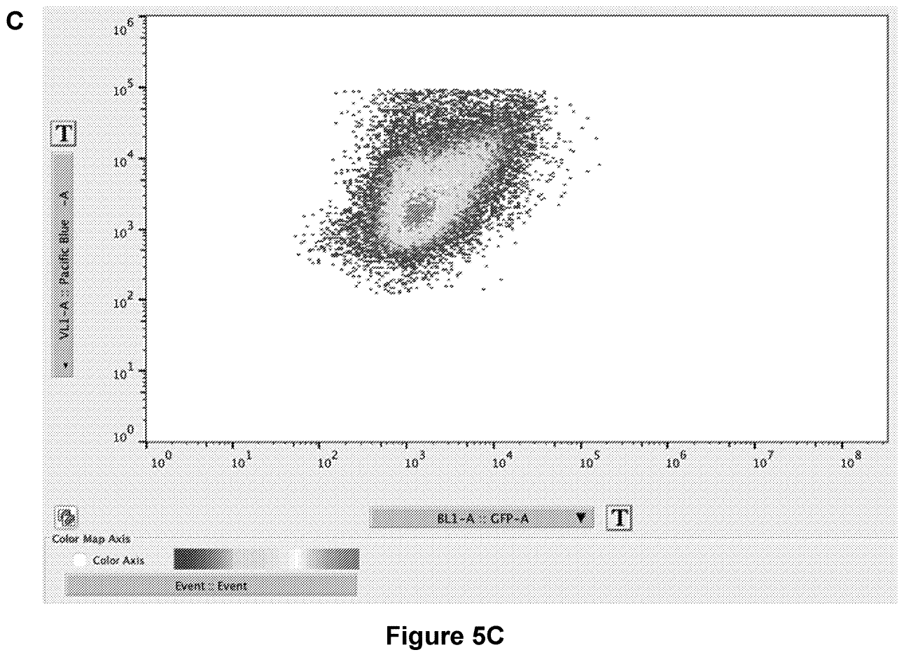

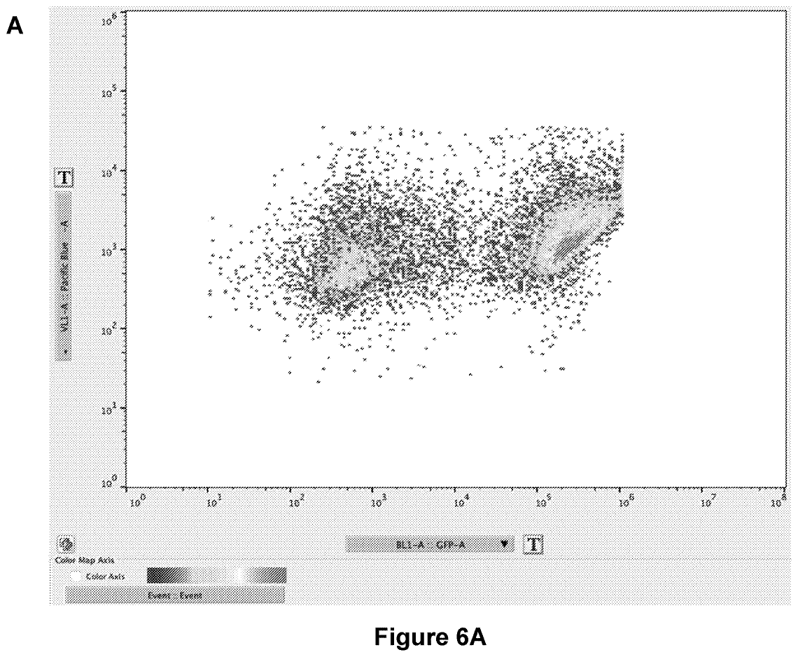

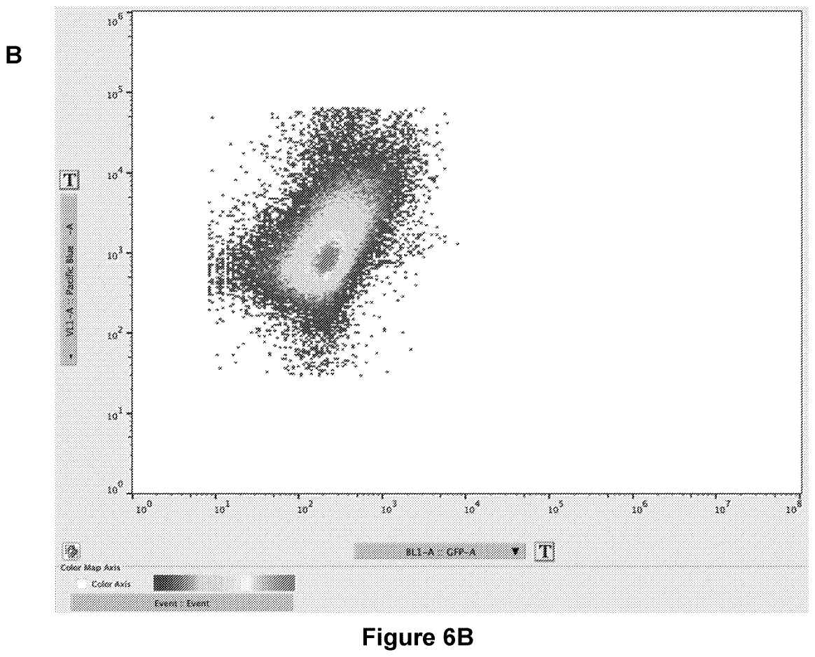

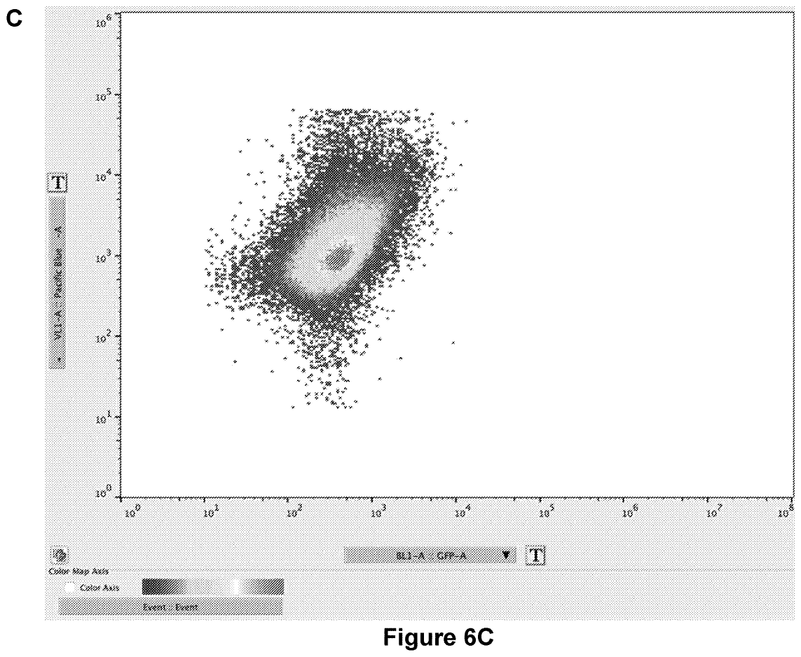

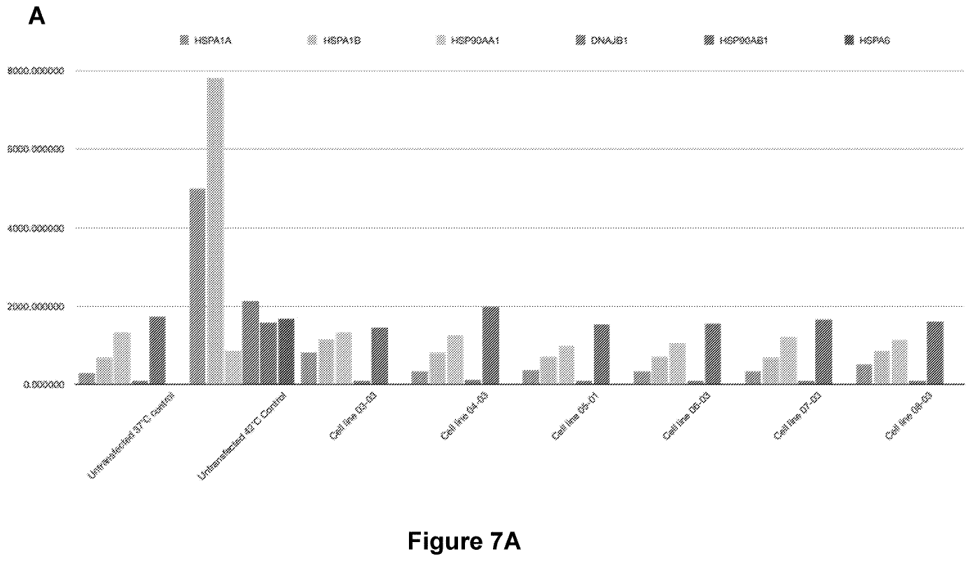

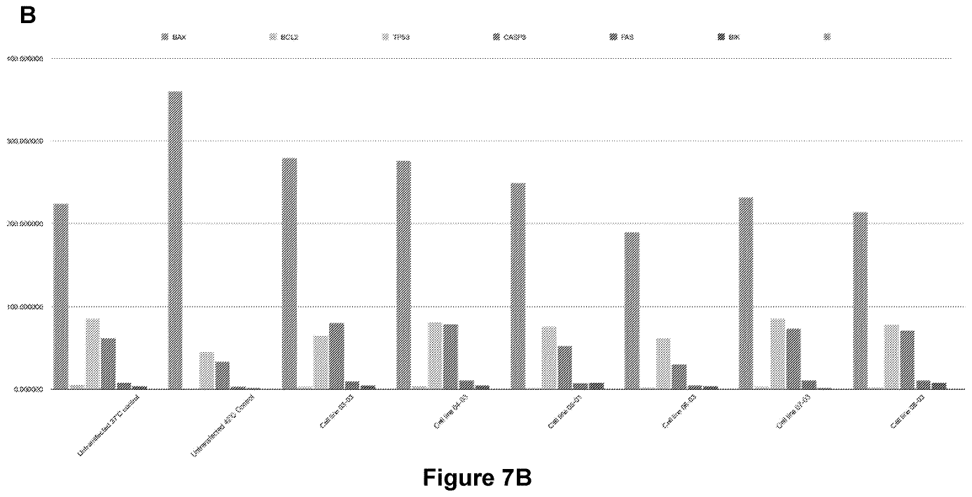

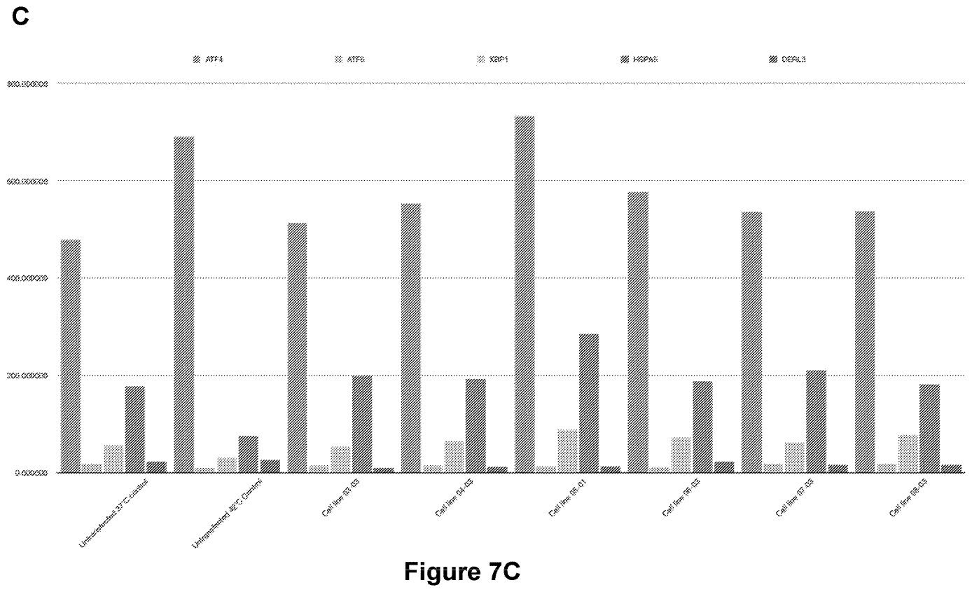

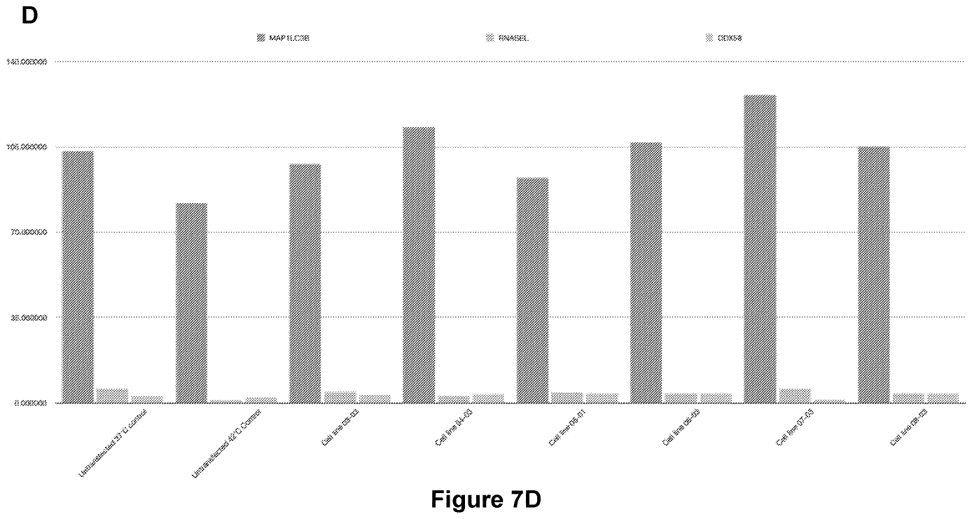

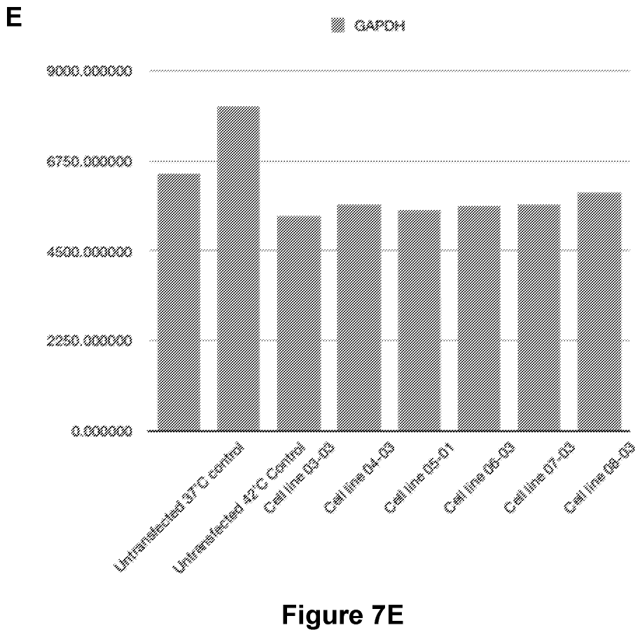

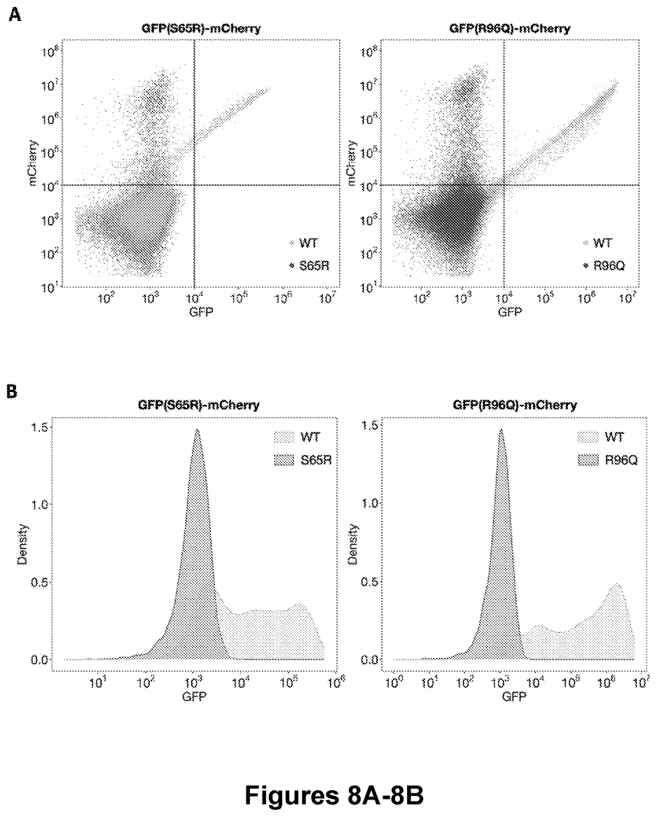

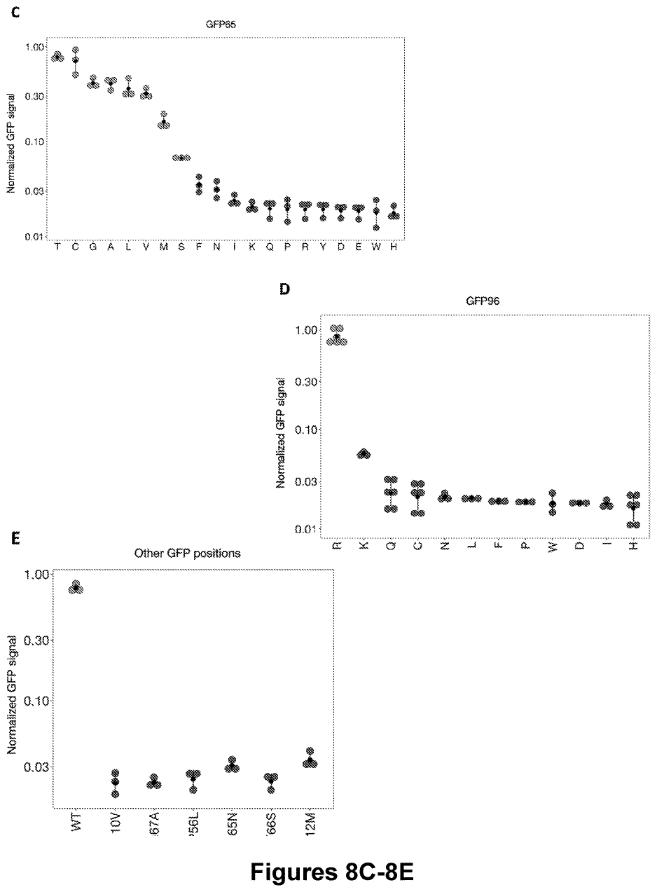

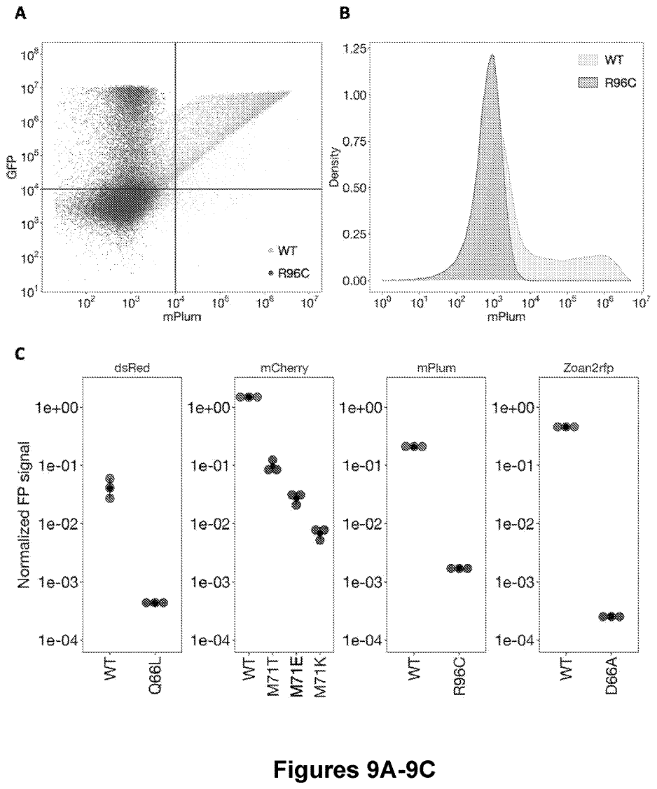

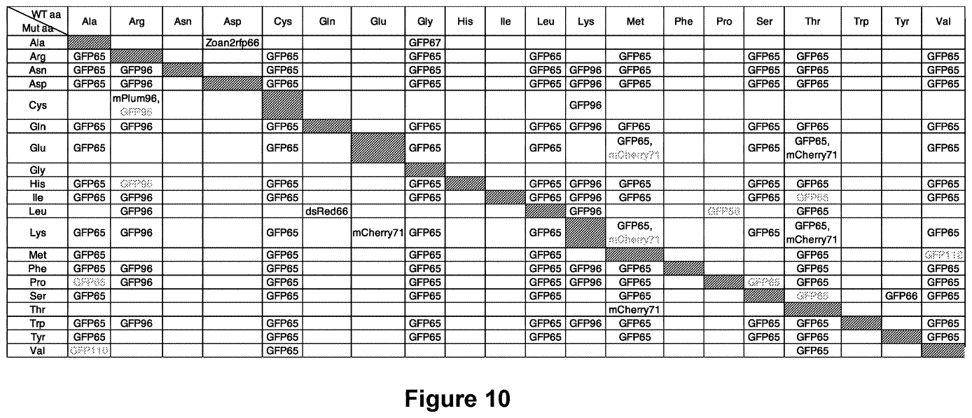

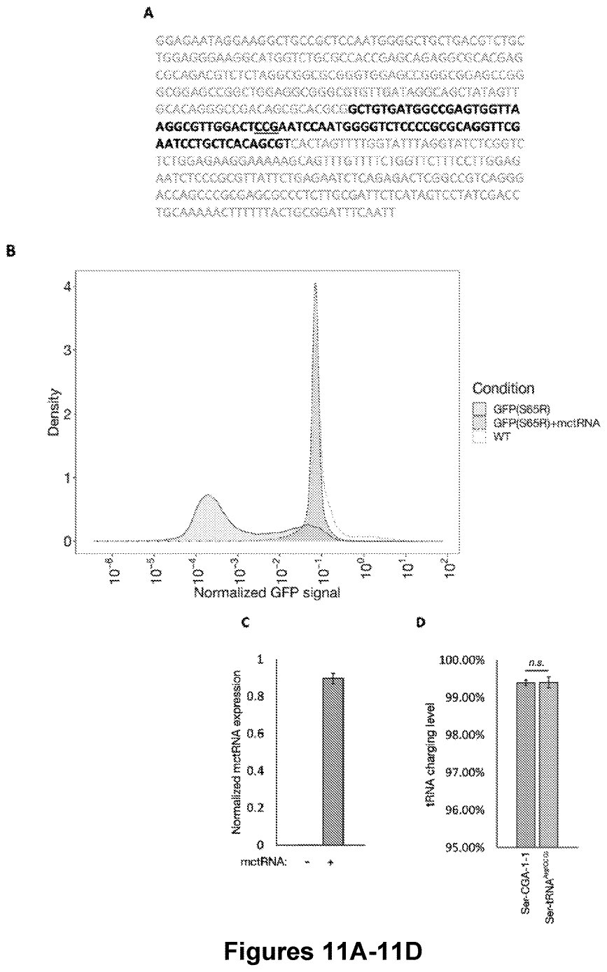

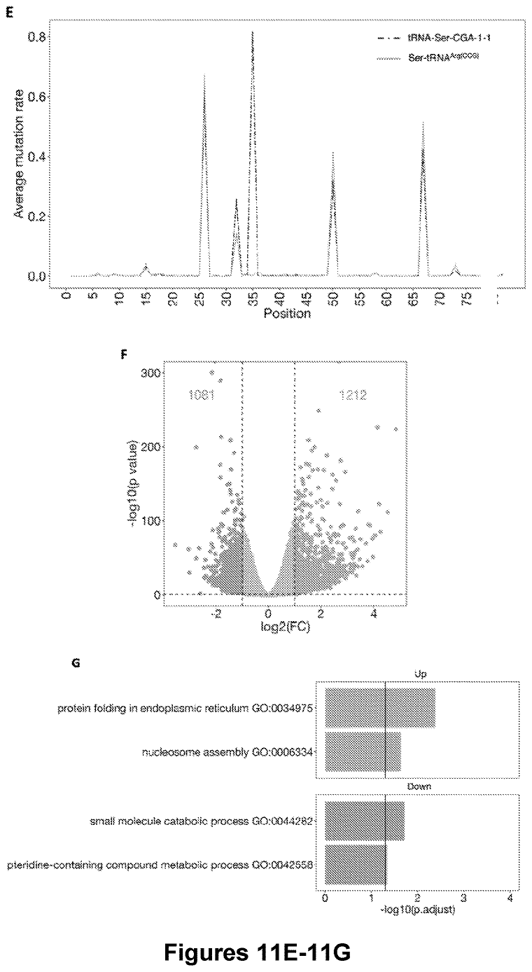

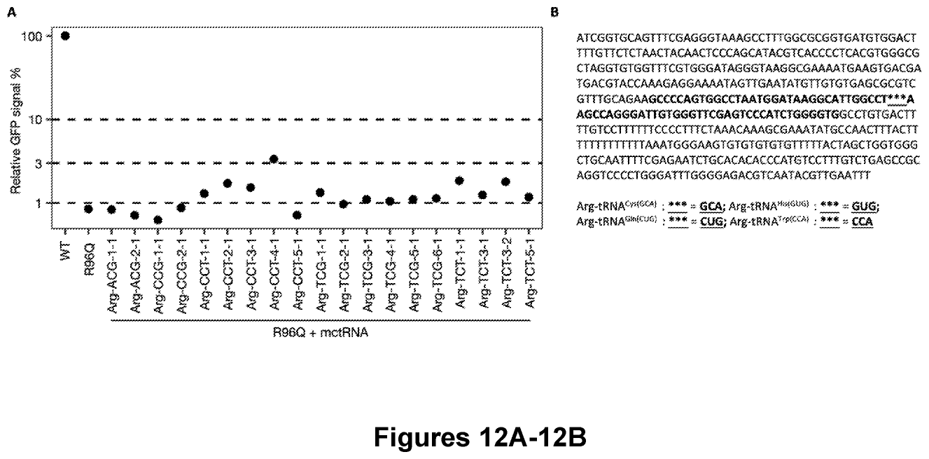

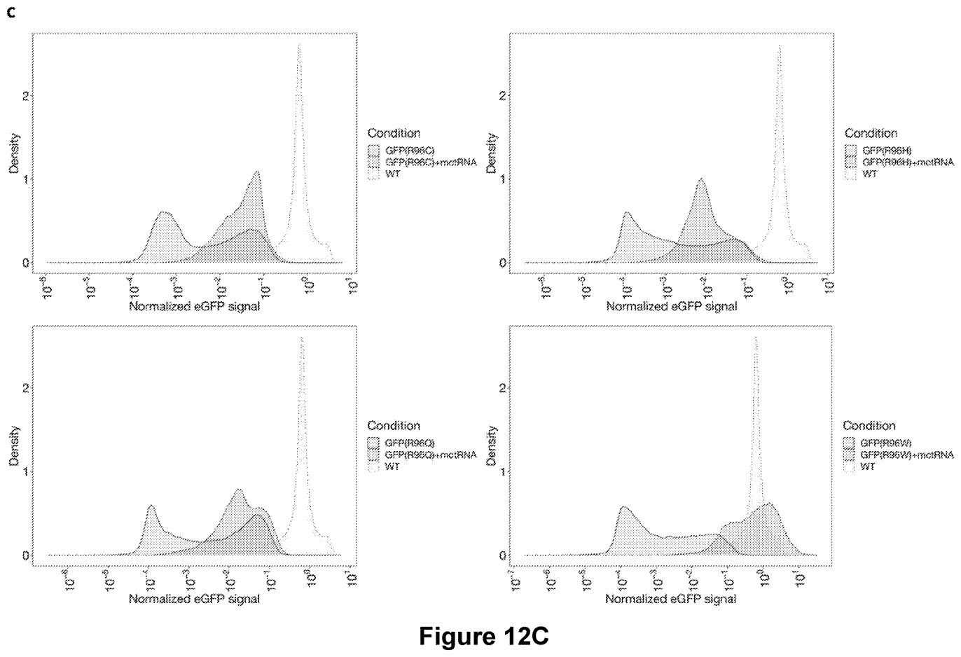

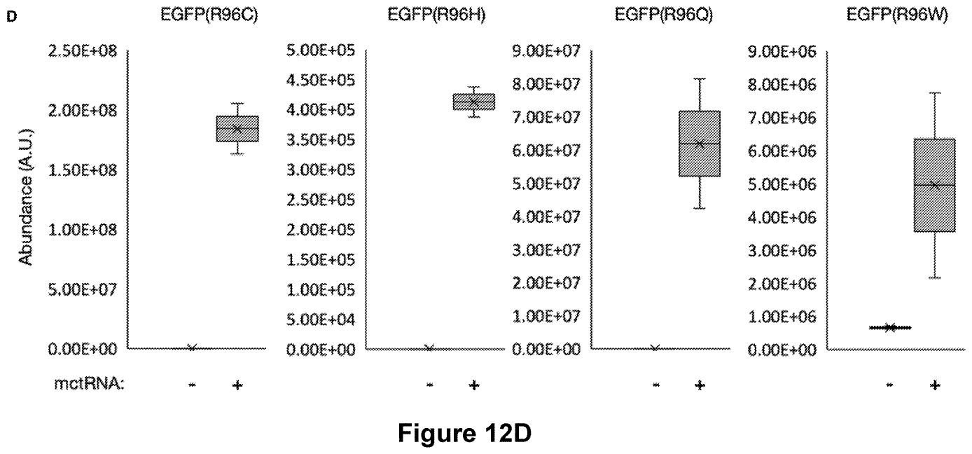

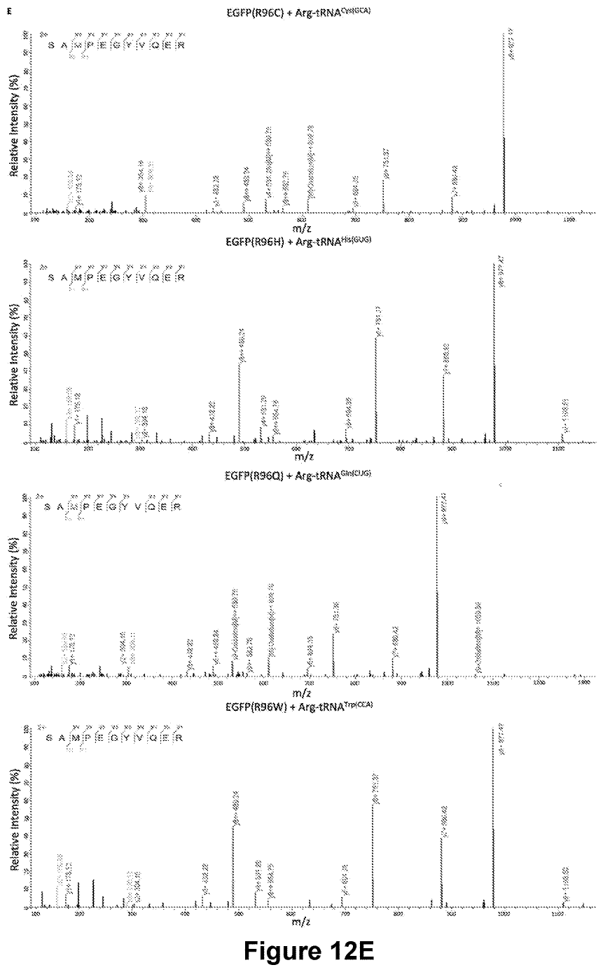

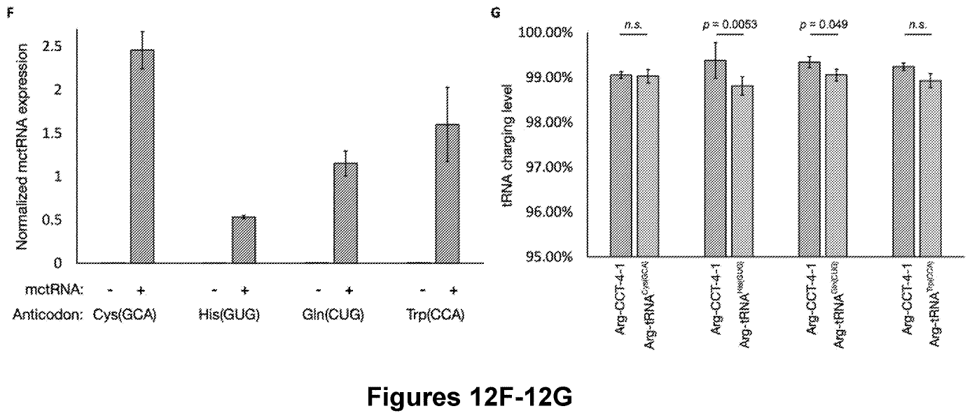

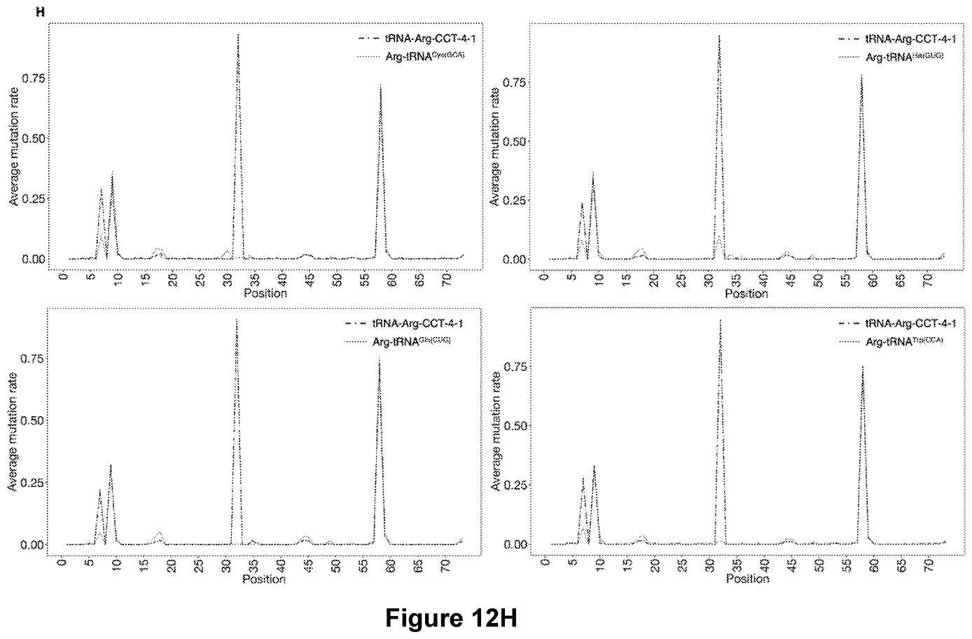

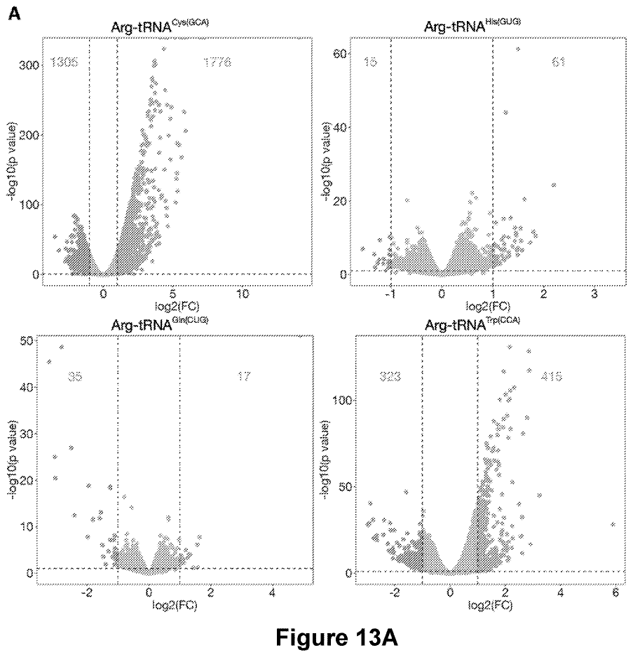

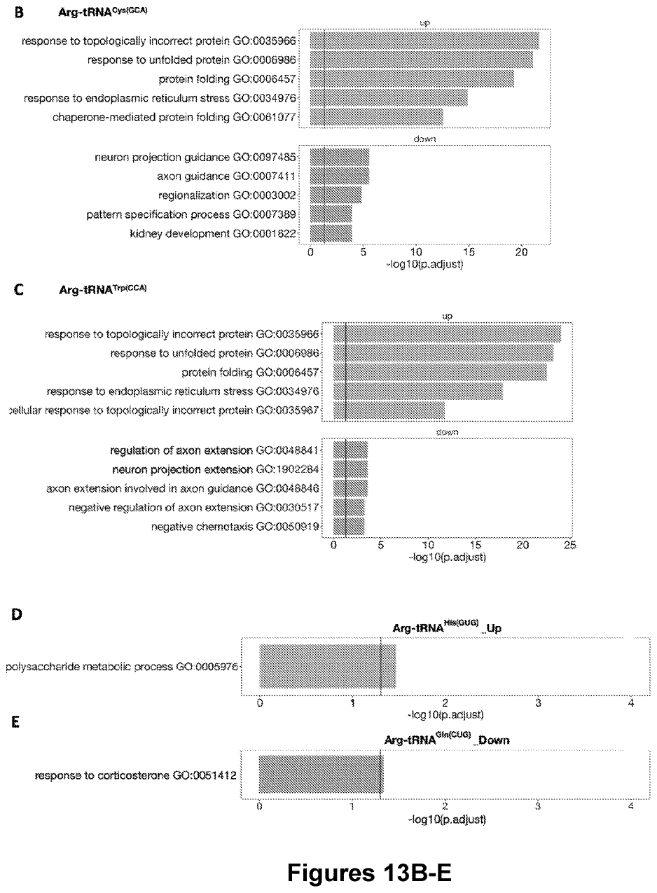

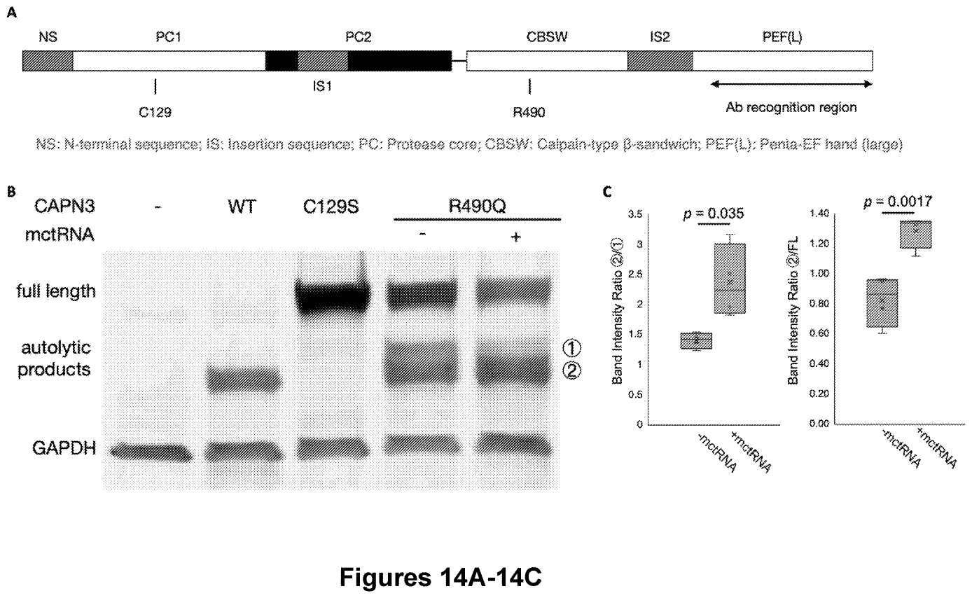

To investigate missense correction to amino acids other than Arg, the inventors designed a similar construct to test for missense correction by MC-tRNA^{Ser}. This construct is also made of a fused GFP and RFP except the GFP contains a mutation of Ser65 to Arg codon of CGG which renders the GFP non-fluorescent (FIG. 4A). The inventors also made several plasmid constructs that contained GFP(Ser65-to-CGG)-RFP (GFP*-RFP) and a MC-tRNA^{Ser}(CCG). MC-tRNA^{Ser}(CCG)1 and MC-tRNA^{Ser}(CCG)2 has a tRNA body sequence derived from a tRNA^{Ser}(AGA) and a tRNA^{Ser}(CGA) isodecoder, respectively. MC-tRNA^{Ser}(CCG)2+ is derived from the same tRNA body sequence of MC-tRNA^{Ser}(CCG)2, but the variable loop has been expanded from 3 to 6 nucleotides. Finally, the inventors also made a negative control construct containing GFP*-RFP and a tRNA^{Ser}(CGA).

The inventors examined the fluorescence levels of GFP and RFP using flow cytometry of HEK293T cells transfected with these plasmids after 24 and 48 hours post-transfection. Cells were gated by RFP fluorescence to validate successful transfection (FIG. 4B). As expected, the negative control of GFP*-RFP plasmid without any additional tRNA shows only red fluorescence (red). The positive control of wild-type GFP-RFP plasmid shows high fluorescence of green and red (green). All three plasmids containing MC-tRNA^{Ser}(CCG) (second, third, and fourth row) show restored green fluorescence at levels within several fold of the positive control GFP-RFP plasmid (FIG. 4C). Finally, the negative control containing tRNA^{Ser}(CGA) (fifth row) did not show restoration of green fluorescence which reinforces the positive results of MC-tRNA^{Ser}.

Although all three MC-tRNA^{Ser}(CCG) restored substantial level of GFP fluorescence, quantitative differences among these constructs highlight the importance of using different tRNA body sequences for missense correction. MC-tRNA^{Ser}(CCG)2 has a narrower distribution and higher fraction of cells with high level of GFP fluorescence compared to that of MC-tRNA^{Ser}(CCG)1, suggesting that MC-tRNA^{Ser}(CCG)2 is a superior missense corrector than MC-tRNA^{Ser}(CCG)1 (FIG. 4D). The expansion of the variable loop in MC-tRNA^{Ser}(CCG)2+ did not show substantial difference to the parent sequence of MC-tRNA^{Ser}(CCG)2, although a literature report suggests that expanding the variable loop can lead to 3-fold higher levels of stop codon suppression (PMID: 20026070). It remains to be seen whether additional change in tRNA body sequences away from those in the reference human genome would generate a more efficient missense corrector. The inventors further explored if expression of MC-tRNAs leads to cell death. It has previously been reported that widespread mistranslation of this type can be cytotoxic. However, using annexin V staining for cell death, the inventors see no increase in cell death in MC-tRNA-expressing cells compared to control cells (FIG. 4E, FIG. 4F). The inventors use the ratio of GFP to RFP expression in MC-tRNA expressing cells as a proxy for tRNA expression level among living cells. The inventors see no correlation between GFP-RFP ratio and annexin V staining (FIG. 4E).

Finally, the inventors searched the literature and identified 18 fluorescence proteins that can be used to study missense correction in the same fashion as Arg and Ser (Table 4). The utilization of these proteins will allow for high throughput screening of efficient MC-tRNAs for every amino acid mutation that leads to a human disease.

### Example 2: Discussion

Potential of the CoMED strategy to treat genetic diseases: In this work the inventors described a strategy of using missense correcting tRNAs to treat genetic mutations that are pathogenic or likely pathogenic in thousands of reported human diseases. The fundamental concept relies on using MC-tRNAs that are charged with one amino acid, but read the codons for another amino acid in translation. The co-translational missense correcting process produces functional proteins, whereas the same proteins translated according to the genetic code would not be functional. The inventors showed that MC-tRNA^{Arg} and MC-tRNA^{Ser} work well to correct their respective missense mutations in a human cell culture, thus providing a proof of principle of the concept of Co-translational missense correction of genetic diseases (CoMED).

In certain aspects, the strategy is well suited to treat genetically recessive diseases. In such cases, genetic mutations produce little or no functional proteins, so that using MC-tRNA can generate a useful amount of functional proteins for disease treatment.

Missense correction and toxicity: A major consideration of introducing MC-tRNA in a cell is the matter of toxicity, as MC-tRNA can also misread other codons in other proteins to increase the level of mistranslation. The typical range of fidelity in the central dogma of molecular biology is 10⁻⁸-10⁻⁹ for replication, 10⁻⁵-10⁻⁶ for transcription, and 10⁻³-10⁻⁴ for translation. Not only does translation have the lowest level of fidelity, cells can actively change the fidelity of translation in response to environmental conditions to broaden their proteome diversity for better response and adaptation (ref.). It is also clear that massive levels of mistranslation can lead to high levels of proteotoxic stress and cell death. The toxic effect will likely depend on many factors such as the cell type (neuron, liver, kidney, etc), the amount of MC-tRNA expression needed for disease treatment, the length of MC-tRNA exposure, and the specific type of the MC-tRNA.

MC-tRNA backbone is not restricted to tRNAs in the reference human genome: In this work the inventors used only tRNA bodies derived from the reference human genome which is derived from pooled DNA of ~100 individuals. However, a large reservoir of other tRNA sequences exists in the human population (PMID: 30643023.), so the number of naturally available tRNA bodies can be readily used for additional tests for MC-tRNA efficacy. Furthermore, tRNA sequences from non-human sources such as mouse/rat, yeast, or even bacterial tRNA could also be tested, they can be functional MC-tRNAs as long as they can be charged with the corresponding human aminoacyl-tRNA synthetase, for example, non-human MC-tRNA^{Arg} chargeable by human arginyl-tRNA synthetase.

tRNA modifications may be needed to fine-tune MC-tRNA efficacy: The innate immune system recognizes unmodified tRNA as foreign, whereas certain modified tRNAs do not trigger an immune response (ref.). Therefore, to achieve the highest efficacy while minimizing immune response, the MC-tRNA can contain specific modifications. tRNA modifications are also highly effective in tuning the co-translational efficiency and selectivity of decoding. A human cytosolic tRNA contains on average 13 modifications per molecule, but not all of these would be needed for maximal efficiency. For example, pseudouridine alone has proven to be highly effective in minimizing immune response in COVID-19 mRNA vaccines. Various modifications and their combinations can be tested to obtain the optimal MC-tRNA constructs with the highest efficacy.

Constellation of 200 tRNAs to treat all single nucleotide missense mutations: In summary, the inventors describe a CoMED strategy of using MC-tRNAs to treat human genetic diseases. the first focus would be using 4-5 MC-tRNA as targeting Arg mutation to Cys/Trp/His/Gln with ~200 potential new patients per year in the US. In principle, a collection of ~200 MC-tRNAs can treat all single nucleotide missense mutations that cause human disease.

### Example 3: Materials and Methods

### MC-tRNA^{Ser} Experiments

MC-tRNA^{Ser} Constructs with Promoters:

Table 1 shows the tRNA sequence and DNA block used for expression of MC-tRNAs. These blocks were cloned into mammalian expression plasmids that also expressed tandem GFP*-RFP polypeptide reporters.

### Transfection Protocol:

HEK293T cells were cultured to confluency. Cells were trypsinized, washed. [Number] of cells were combine with [ng of plasmid] and [lipofectamine] for transfection. After [incubation] cells were washed and grown at [temperature] for [time].

### Staining Protocol:

When indicated, cells were stained with either DAPI or annexin-V AlexaFluor 647 or both as follows: [DAPI]. [Annexin-V]

### Flow Cytometry Protocol:

At 24, 48, and 72 hours post transfection cells were trypsinized in 96 well plates with [Trypsen reagent]. Cells were pelleted, decanted, washed with PBS, and resuspended in 200 uL of PBS. Cells were then analyzed on the [INSTURMENT] with [LASERS]. Instrument default voltages were used. Forward scatter and side scatter parameters were used to gate for living cells and 10k events in this gate were recorded. Subsequent analysis was done with custom scripts in R using the FlowCore and FlowVis CRAN packages.

### ClinVar Computation

ClinVar data was downloaded Jan. 4, 2022 (https://ftp.ncbi.nlm.nih.gov/pub/clinvar/tab_delimited/) (PubMed PMID:31777943). Analysis was done with custom scripts in R.

Comparison of Missense Vs. Nonsense SNVs Among all SNVs

The dataset of variant summary from ClinVar was filtered for Assembly=“hg38”, type=“single nucleotide variant”. The name field was parsed to get WT and SNV amino acid identity and WT nucleotide and SNV nucleotide. If the SNV amino acid was “Ter”, indicating terminator, this was labeled a nonsense mutation, otherwise the entry was labeled missense. The number of reported SNVs for both missense and nonsense was summed.

### Comparison of Pathogenic SNVs Among Amino Acids

The parsed set of SNVs from above was further filtered for ClinicalSignificance field, accepting “pathogenic”, “likely pathogenic”, and “pathogenic/likely pathogenic”. This filtered set was used as the set of pathogenic SNVs. Grouping SNVs by the wild type amino acid, the number of SNV entries in each group was summed for comparison.

### Comparison of Number of Submitters Per SNV

In the filtered set of pathogenic SNVs from above, the NumberSubmitters field was used to make a histogram reflecting how frequently individual SNVs are reported from the population.

### Comparison of Number of SNVs and Submitters Per SNV for Missense Pathogenic Mutations

The PhenotypeIDS field was parsed to identify Orphanet IDs associated with each SNVs. SNVs were then grouped by their mutation type—a combination of WT amino acid, WT nucleotide, SNV amino acid, and SNV nucleotide. This identifying information is a proxy for codon level information—though each combination can be matched to a small group of possible codons. Using these groups, the NumberSubmitter for each SNV was summed to yield “total_reports”. Additionally, the list of unique PhenotypeIDS entries was tallied to track “number_diseases” associated with each SNV type. The number of submitters of each SNV was also tracked. For plotting, names are only displayed for SNV types where at least one individual SNV has more than 10 submitters.

### Example 4: Details on CAPN3 Gene Related to Disease LGMD2A and Missense Mutations

The disease Limb Girdle Muscular Dystrophy type 2A, LGMD2A is related to mutations in the gene Caplain3, CAPN3. Generally, this disease is considered genetically recessive. The inventors postulate that restoration of a small amount of the enzymatic function of CAPN3 would be sufficient to improve symptoms of patients suffering from LGMD2A, thus tRNA therapy is a good candidate. This disease was chosen as a good candidate for commercial development for 6 reasons: 1) Because CAPN3 is an enzyme and the disease presents as genetically recessive, a tRNA therapy is likely to be efficacious 2) there is evidence that gene therapy has safety concerns based on off-target cardiac delivery of the CAPN3 gene 3) The disease is non-lethal, thus there is a population of adult patients who can be enrolled in clinical trials without waiting for diagnoses 4) The patient population, while small, is potentially large enough to support therapeutic development, with estimates at 1 per 100,000 births in the US 5) the tissues affected are skeletal muscle which have many viable delivery options 6) there are convenient assays to test for gene function, e.g. auto proteolysis.

This disease and SNVs appear in ClinVar so the inventors analyzed the known pathogenic and likely pathogenic SNVs related to the disease. The inventors found that 65.2% of SNVs are missense mutations, compared to 34.8% for nonsense mutations (there are mutations that affect splicing or frameshift, which were excluded from this analysis). Among the missense mutations, Arg>Gln accounted for 24% of reported cases; Arg>Trp 12%; Arg>Cys 4%; Arg>His 3%—this trend is consistent with analysis appearing herein indicating that mutations from Arg account for a plurality of pathogenic missense mutations among all ClinVar diseases. While every individual disease is unique, the landscape of SNVs in the human population for LGMD2A follows this same trend. While Arg>Gln mutations account for the largest single type of mutation, this includes many different SNVs, all of which could plausibly be treated with an mc-tRNA. Supplemental tables are included articulating the precise SNVs reported in Clinvar for CAPN3, as well as statistics included here for combine fraction of Arg>Gln mutations, and fraction of missense vs. nonsense mutations.

To test if mc-tRNAs can truly restore function to CAPN3, the inventors purchased plasmids expressing mutant CAPN3, which are expected to be dysfunctional in a well documented auto-proteolytic activity, compared to wild type. These CAPN3 mutants would be expressed in HEK293 cells, and co-expressed with mc-tRNA to repair their corresponding mutation. The inventors chose CAPN3 mutations that are either biochemically verified in the literature, or confirmed to exist in patients of LGMD2A by the Coalition to Cure Calpain 3. The inventors performed control western blots with HEK293 cells expressing CAPN3 or mutant CAPN3, but not the mc-tRNA-expressing constructs.

### Example 5: Generation of Stable Cells Lines Expressing GFP.RFP, or MutantGFP.RFP, or Mutant GFP.RFP and a Corrective tRNA

### Stable Cell Line Generation

HEK293 cells were purchased from ATCC and cultured using standard sterile mammalian cell culture practices. In more detail, HEK293 cells were maintained in culture in 10 cm polystyrene cell culture treated dishes in DMEM supplemented with 10% FBS, Penicillin, and Streptomycin, at 37′C in a humid, 5% CO2 atmosphere. To generate stable cell lines expressing the proteins and tRNAs of interest, HEK293 cells were split into and subcultured in 6 well cell culture treated plates, transfected with a plasmid expressing the desired protein/tRNA of interest and a selectable marker, and then selected using antibiotic based selection. The goal of this project was to study mutation correction and stress in cells constitutively expressing tRNA engineered to recognize and correct specific missense mutations (mc-tRNA).

For transfection, healthy HEK293 cells were first seeded into 6 well cell culture treated plates at a density of 900,000 cells/well in DMEM supplemented with only 10% FBS. Cells were allowed to adhere to the well for 24 hours. After 24 hours the cells were transfected using lipofectamine 2000 and the plasmids of interest. In more detail, for each well Sul of lipofectamine 2000 reagent was combined with 2 ug of plasmid of interest in 500 ul OptiMEM and incubated for 20 min at room temperature. The combined lipofectamine/plasmid transfection reagent was then added dropwise to the appropriate well and mixed by swirling the well 20× clockwise and counter clockwise. Transfected cells were incubated for 4 hour at 37′C in a humid, 5% C02 atmosphere. After 4 hours the media was removed from each well and replaced with new DMEM supplemented with 10% FBS, Penicillin, and Streptomycin. All transfected plasmids contain an ampicilin and a puromycin resistance cassette.

The following plasmids were transfected:
p2—A positive control plasmids expressing an eGFP.mCherry fusion protein from a CMV promoter. Both fluorescent proteins functional.
p100—A negative control expressing an eGFP.mCherry fusion protein containing a mutant eGFP. This mutant eGFP has a R to Q amino acid change at position 97 (R97Q). This mutant will be here in described as eGFP(R97Q).mCherry. The codon for Q at this position is CAG. This mutation prevents eGFP from being fluorescent, but the RFP protein is still fluorescent.
p108—A test plasmid expressing eGFP(R97Q).mCherry and an mc-tRNA which recognizes the mutated CAG codon but delivers an Arginine. This tRNA had previously been shown to correct the mutation in eGFP in transient transfection based experiments.
p401—A negative control expressing an eGFP.mCherry fusion protein containing a mutant eGFP. This mutant eGFP has a R to C amino acid change at position 97 (R97C). This mutant will be here in described as eGFP(R97C).mCherry. The codon for C at this position is TGC. This mutation in eGFP greatly diminishes its fluorescence, but the RFP protein is still fully fluorescent.
p404—A test plasmid expressing eGFP(R97C).mCherry and an mc-tRNA which recognizes the mutated TGC codon but delivers an Arginine. This tRNA had previously been shown to correct the mutation in eGFP in transient transfection based experiments.

In total 2×6 well plates of cells were transfected. The first plate was transfected with plasmids p2, p100, p103, and p108. The second plate was transfected with plasmids p2, p401, and p404. Additional untransfected wells were maintain to provide selection controls.

Transfected cells were allowed to grow and express the transfected constructs for 48 hours at 37′C in a humid, 5% C02 atmosphere. After 48 hours cells were selected using 0.5 ug/ml puromycin. Cells were maintained in media containing 0.5 ug/ml puromycin for 2 weeks with media changes ~every 2-3 days. After 2 weeks in selection the cells which had stably integrated the plasmid into their genome were still alive and growing. These stable cells were expanded into 10 cm cell culture dishes and maintaining in DMEM supplemented with 10% FBS, penicillin, streptomycin, and 0.3 ug/ml puromycin, at 37′C in a humid, 5% C02 atmosphere.

The following stable cells lines were generated:
Cell line 03-00—HEK293 cells transfected with p2, expressing WT eGFP.mCherry, polyclonal, resistant to at least 0.5 ug/ml puromycin
Cell line 04-00—HEK293 cells transfected with p401, expressing eGFP(R97C).mCherry, polyclonal, resistant to at least 0.5 ug/ml puromycin
Cell line 05-00—HEK293 cells transfected with p404, expressing eGFP(R97C).mCherry and an mc-tRNA Arg-tRNA-CysGCA, polyclonal, resistant to at least 0.5 ug/ml puromycin
Cell line 06-00—HEK293 cells transfected with p2, expressing WT eGFP.mCherry, polyclonal, resistant to at least 0.5 ug/ml puromycin
Cell line 07-00—HEK293 cells transfected with p100, expressing eGFP(R97Q).mCherry, polyclonal, resistant to at least 0.5 ug/ml puromycin
Cell line 08-00—HEK293 cells transfected with p108, expressing eGFP(R97Q).mCherry and an mc-tRNA Arg-tRNA-GlnCUG, polyclonal, resistant to at least 0.5 ug/ml puromycin
Note: Cell line nomenclature is as follows, line number—split number. Ex: 06-03 is cell line 6, split 3.

Of note, stable cell line 05, transfected with plasmid p404, grew very slowly in comparison to the other cell lines. The inventors believe this is due to the large suppression effect this mc-tRNA has, recovering 10% of eGFP fluorescence in transient transfection based experiments. Additionally, the positive control lines (transfected with p2) from both plates survived selection, leading to the creating of two polyclonal positive control lines (03-00 and 06-00).

### Fluorescence Recovery

Fluorescence recovery was examined using flow cytometry. Stable cell lines were prepared for flow cytometry as follows. Stable cells were seeded into a 6 well dish at 300,000 cells/well and allowed to grow for 24 hours. The media was then removed and each well was washed with 1 ml of 1×PBS. The PBS was then removed and 500 ul of 0.25% Trypsin:EDTA was added to each well, and incubated for 5 min at RT. Once incubation was complete, 500 ul of DMEM supplemented with 10% FBS was added to each well. The cells were then resuspended by pipetting, moved to a 1.7 ml centrifuge tube, and sedimented by centrifugation at 5000×g for 5 min. The supernatant was removed from the sedimented cells, with care taken to to disturb the cell pellet, and cells were resuspended in 500 ul 1×PBS. Cells were then stained wth DAPI to identify dead vs alive cells. DAPI staining was performed by adding 10 ul DAPI (10 ug/ml stock) to the 500 ul of resuspended cells, incubating for 15 min at RT. After incubation an additional 500 ul 1×PBS was added to the resuspended cells. Cells were kept on ice until use.

Fluorescence was measure using an Attune Flow Cytometer. GFP and DAPI signal was measured from all stable cells lines. GFP signal was used as a measure of missense suppression by the mc-tRNA. DAPI signal was used as a measure of cell death from the expression of the mc-tRNA.

### Next Generation Sequencing

Total RNA was isolated from stable cell lines using standard a Trizol:Chloroform extraction procedure. mRNA was isolated and DNA libraries constructed using the Illumina Stranded mRNA prep kit. Libraries were sequenced on an Illumina MiSeq system.

### Example 6: Further Cell Culture Data

### Mammalian Cell Culture

HEK293 cells were authenticated and tested negative for mycoplasma. HEK293 cells were cultured at 37° C. with 5% CO2 in Dulbecco's Modified Eagle's (DMEM) high-glucose medium (Cytiva Cat. SH30022.01) supplemented with 10% heat-inactivated fetal bovine serum and 100 U/ml Penicillin-Streptomycin.

### Plasmids

Plasmids were synthesized and validated by GenScript, inc. Fluorescence protein-based mistranslation reporters were driven by CMV promoter. MctRNA expression cassettes contained 200 bp endogenous sequences upstream as well as downstream of the tRNA genes. The exact sequences are as follows:

(1) Expression cassette for Ser-tRNA^{Arg(CCG)}
(SEQ ID NO: 140):
GGAGAATAGGAAGGCTGCCGCTCCAATGGGGCTGCTGACGTCTG
CTGGAGGGAAGGCATGGTCTGCGCCACCGAGCAGAGGCGCACGA
GCGCAGACGTCTCTAGGCGGCGCGGGTGGAGCCGGGCGGAGCCG
GGCGGAGCCGGCTGGAGGCGGGCGTGTTGATAGGCAGCTATAGT
TGCACAGGGCCGACAGCGCACGCGGCTGTGATGGCCGAGTGGTT
AAGGCGTTGGACTCCGAATCCAATGGGGTCTCCCCGCGCAGGTT
CGAATCCTGCTCACAGCGTCACTAGTTTTGGTATTTAGGTATCT
CGGTCTCTGGAGAAGGAAAAAGCAGTTTGTTTTCTGGTTCTTTC
CTTGGAGAATCTCCCGCGTTATTCTGAGAATCTCAGAGACTCGG
CCGTCAGGGACCAGCCCGCGAGCGCCCTCTTGCGATTCTCATAG
TCCTATCGACCTGCAAAAACTTTTTTACTGCGGATTTCAATT
(2) Expression cassette for Arg-tRNA^{Cys(GCA)}
(SEQ ID NO: 141):
ATCGGTGCAGTTTCGAGGGTAAAGCCTTTGGCGCGGTGATGTGG
ACTTTTGTTCTCTAACTACAACTCCCAGCATACGTCACCCCTCA
CGTGGGCGCTAGGTGTGGTTTCGTGGGATAGGGTAAGGCGAAAA
TGAAGTGACGATGACGTACCAAAGAGGAAAATAGTTGAATATGT
TGTGTGAGCGCGTCGTTTGCAGAAGCCCCAGTGGCCTAATGGAT
AAGGCATTGGCCTGCAAAGCCAGGGATTGTGGGTTCGAGTCCCA
TCTGGGGTGGCCTGTGACTTTTGTCCTTTTTTCCCCTTTCTAAA
CAAAGCGAAATATGCCAACTTTACTTTTTTTTTTTTTAAATGGG
AAGTGTGTGTGTGTTTTTACTAGCTGGTGGGCTGCAATTTTCGA
GAATCTGCACACACCCATGTCCTTTGTCTGAGCCGCAGGTCCCC
TGGGATTTGGGGAGACGTCAATACGTTGAATTT
(3) Expression cassette for Arg-tRNA^{His(GUG)}
(SEQ ID NO: 142):
ATCGGTGCAGTTTCGAGGGTAAAGCCTTTGGCGCGGTGATGTGG
ACTTTTGTTCTCTAACTACAACTCCCAGCATACGTCACCCCTCA
CGTGGGCGCTAGGTGTGGTTTCGTGGGATAGGGTAAGGCGAAAA
TGAAGTGACGATGACGTACCAAAGAGGAAAATAGTTGAATATGT
TGTGTGAGCGCGTCGTTTGCAGAAGCCCCAGTGGCCTAATGGAT
AAGGCATTGGCCTGTGAAGCCAGGGATTGTGGGTTCGAGTCCCA
TCTGGGGTGGCCTGTGACTTTTGTCCTTTTTTCCCCTTTCTAAA
CAAAGCGAAATATGCCAACTTTACTTTTTTTTTTTTTAAATGGG
AAGTGTGTGTGTGTTTTTACTAGCTGGTGGGCTGCAATTTTCGA
GAATCTGCACACACCCATGTCCTTTGTCTGAGCCGCAGGTCCCC
TGGGATTTGGGGAGACGTCAATACGTTGAATTT
(4) Expression cassette for Arg-tRNA^{Gln(CUG)}
(SEQ ID NO: 143):
ATCGGTGCAGTTTCGAGGGTAAAGCCTTTGGCGCGGTGATGTGG
ACTTTTGTTCTCTAACTACAACTCCCAGCATACGTCACCCCTCA
CGTGGGCGCTAGGTGTGGTTTCGTGGGATAGGGTAAGGCGAAAA
TGAAGTGACGATGACGTACCAAAGAGGAAAATAGTTGAATATGT
TGTGTGAGCGCGTCGTTTGCAGAAGCCCCAGTGGCCTAATGGAT
AAGGCATTGGCCTCTGAAGCCAGGGATTGTGGGTTCGAGTCCCA
TCTGGGGTGGCCTGTGACTTTTGTCCTTTTTTCCCCTTTCTAAA
CAAAGCGAAATATGCCAACTTTACTTTTTTTTTTTTTAAATGGG
AAGTGTGTGTGTGTTTTTACTAGCTGGTGGGCTGCAATTTTCGA
GAATCTGCACACACCCATGTCCTTTGTCTGAGCCGCAGGTCCCC
TGGGATTTGGGGAGACGTCAATACGTTGAATTT
(5) Expression cassette for Arg-tRNA^{Trp(CCA)}
(SEQ ID NO: 144):
ATCGGTGCAGTTTCGAGGGTAAAGCCTTTGGCGCGGTGATGTGG
ACTTTTGTTCTCTAACTACAACTCCCAGCATACGTCACCCCTCA
CGTGGGCGCTAGGTGTGGTTTCGTGGGATAGGGTAAGGCGAAAA
TGAAGTGACGATGACGTACCAAAGAGGAAAATAGTTGAATATGT
TGTGTGAGCGCGTCGTTTGCAGAAGCCCCAGTGGCCTAATGGAT
AAGGCATTGGCCTCCAAAGCCAGGGATTGTGGGTTCGAGTCCCA
TCTGGGGTGGCCTGTGACTTTTGTCCTTTTTTCCCCTTTCTAAA
CAAAGCGAAATATGCCAACTTTACTTTTTTTTTTTTTAAATGGG
AAGTGTGTGTGTGTTTTTACTAGCTGGTGGGCTGCAATTTTCGA
GAATCTGCACACACCCATGTCCTTTGTCTGAGCCGCAGGTCCCC
TGGGATTTGGGGAGACGTCAATACGTTGAATTT

### Transfection

Cells were seeded on 96- or 6-well plates one day before transfection and reached 50-70% confluent on the day of transfection. Plasmids used for transfection were prepared with PureYield™ plasmid Miniprep/Midiprep systems (Promega Cat. A1222/A2492) and ethanol precipitated. Transfections were performed using Lipofectamine™ 3000 (Invitrogen™ Cat. L3000015) and following the manufacture's protocol.

### Flow Cytometry

Flow cytometry assays were conducted 48 h post-transfection. HEK293 cells were dissociated from the plates with 0.25% Trypsin-EDTA (Gibco™ Cat. 25200056) and resuspended in ice-cold 1×PBS, 5 mM EDTA, 25 mM HEPES pH 7.0, 1% FBS and 100 ng/ml DAPI before sorting. The flow cytometer instrument was NovoCyte Penteon 5-30. Fluorescent protein (FP) signals were detected with cognate detection channels as follows: GFP, B525; mCherry, Y615; dsRed/Zoan2rfp, Y586; mPlum, Y667. For every flow cytometry run, wild type HEK293 cells without any stain were used as background control, and HEK293 cells overexpressing single FP were used as gating references. Collected flow cytometry data were analyzed using a custom R script.

### Immunoprecipitation

HEK293 cells were harvested 48 h post-transfection and washed twice with 1×PBS. 3~7 million cells were then incubated in 500 μl of lysis buffer (1×PBS, 1% IGEPAL (NP-40), 0.1% SDS, 0.5% w/v sodium deoxycholate, 1× protease inhibitor cocktail (Nacalai Cat. 25955)) on a rotator at 4° C. for 15 min and centrifuged at 1,000 g, 4° C. for 5 min to pellet the cellular debris. The GFP-mCherry fusion protein was immunoprecipitated (IP) with GFP monoclonal antibody (Invitrogen Cat. MA515256) and CAPN3 was IPed with CAPN3 monoclonal antibody (Proteintech® Cat. 67366-1-Ig). Antibodies were incubated with pre-washed Dynabeads™ M-280 Sheep anti-Mouse IgG beads (Cat. 11202D) at 4° C. for 4 h before adding to the cell lysates. The ratio of antibody to beads is 4 μg to 50 μl and the dilution of antibody in the cell lysate is 1 μg in 50 μl. Antibody-conjugated beads were washed 3 times with 1 ml lysis buffer and added to the cell lysates. The mixtures were incubated at 4° C. overnight. Subsequently, beads were washed 3 times with 1 ml high salt wash buffer (50 mM Tris-HCl pH7.4, 1M NaCl, 1 mM EDTA, 1% IGEPAL (NP-40), 0.1% SDS, 0.5% w/v sodium deoxycholate, 1× protease inhibitor cocktail) and 3 times with 1 ml low salt wash buffer (20 mM Tris-HCl pH7.4, 10 mM MgCl₂, 0.2% Tween-20, 1× protease inhibitor cocktail). The IPed proteins were eluted from the beads by incubating in non-reducing 1× NuPAGE LDS sample buffer (Invitrogen™ Cat. NP0007) at 70° C. for 15 min and directly loaded on 4-12% NuPAGE gels (Invitrogen™ Cat. NP0321BOX) for gel electrophoresis. NuPAGE gels were stained with Coomassie G-250 stain (Cat. #1610786) and target bands were cut and stored at 4° C.

### Mass Spectrometry Sample Preparation

Protein spots were extracted from an SDS-PAGE gel and cut into 1 mm×1 mm cubes. The in-gel digestion process was performed following published protocols with some modifications 1. First, the gel pieces were dehydrated using acetonitrile and the solution was removed after 5 minutes. Then, the gel pieces were covered with a sufficient volume of a solution containing 5 mM TCEP in 40 mM ammonium bicarbonate with 25% acetonitrile. The mixture was incubated at 37° C. for 5 minutes to allow for sufficient reduction of disulfide bridges in the proteins. The gel pieces were dehydrated again using acetonitrile, followed by the addition of another solution of 5 mM TCEP in 40 mM ammonium bicarbonate with 25% acetonitrile. This step aimed to further reduce the disulfide bridges and was incubated at 65° C. for 15 minutes with gentle agitation. After cooling to room temperature, the gel pieces were treated with 40 mM iodoacetamide in 40 mM ammonium bicarbonate with 25% acetonitrile for alkylation. The gel pieces were incubated in the dark at room temperature for 15 minutes. Subsequently, the gel pieces were washed with 40 mM ammonium bicarbonate with 25% acetonitrile for 5 minutes, followed by dehydration using acetonitrile. This wash step was repeated once more. The gel pieces were then swollen in a digestion buffer containing trypsin/Lys-C (8 ng/μL) in 40 mM ammonium bicarbonate and 0.5 mM CaCl₂ with the use of an ice-cold bath for 1 hour. The excess trypsin solution was removed from the gel pieces while keeping them on ice. The samples were digested for 12 hours at 37° C. To extract the peptides, two changes of 0.2% formic acid and one change of 0.15% formic acid in 60% acetonitrile were used, with incubation times of 10 minutes and 30 minutes, respectively. Finally, the peptides were dried in vacuo.

### LC-MS Parameters

The samples underwent analysis using an Exploris 480 mass spectrometer connected to an UltiMate 3000 liquid chromatography system (Thermo Scientific). The chromatography system utilized a MonoCap column from GL Sciences, measuring 50 cm in length and 0.75 mm in inner diameter (Cat. No. 5020-10006). The flow rate was maintained at 500 nL/min, and the temperature was kept constant at 25° C. A gradient method spanning 75 minutes was employed, involving mobile phase A (0.15% formic acid in water) and mobile phase B (0.15% formic acid in 100% acetonitrile). The gradient proceeded as follows: 5% B for 5 minutes, followed by a transition from 5% to 22% B over 46.5 minutes, a transition from 22% to 34% B over 7.5 minutes, and a rapid transition from 30% to 95% B in 1 minute. The composition was maintained at 95% B for 4 minutes. A full-scan MS spectrum ranging from 350 to 1650 m/z was collected at a resolution of 120,000 at m/z 200. The maximum injection time was set to 50 ms, and the AGC target value was set to 3e6. The cycle time for data acquisition was set to 3 seconds, while the intensity threshold was set at 5e4. For MS/MS scans, a resolution of 15,000 was employed, with the maximum acquisition time set to auto and an AGC target value of 4e4. The isolation window at the Orbitrap cell was set to 1.6 m/z, and the first mass was set to 110 m/z. The collision energy for HCD was set to 32. A dynamic exclusion duration of 10 seconds was implemented, and charge states of unassigned, 1, and 8 or greater were excluded. The heated capillary temperature was set to 300° C.

### MS Data Analysis

Raw MS data were processed and searched with Proteome Discoverer (version 3.0.0.757; Thermo Fisher Scientific) using the Sequest HT search engine. The precursor mass tolerance of 10 ppm and fragment mass tolerance of 0.02 Da were used. 1% FDR cutoff was applied to filter the data, which was estimated by Target Decoy PSM Validator. Trypsin (full) was set as the enzyme in the search. The maximum mass cleavages were set to 3, and the peptide lengths were from 4 to 30. Carbamidomethyl (+57.021 Da on C) was selected as fixed modification whereas oxidation (+15.995 Da on M), Deamidated (+0.984 Da on N and Q), protein N-terminal Met-loss (−131.040 Da), Acetyl (+42.011 Da on N-terminus), and Protein N-terminal Met-loss+Acetyl (−89.030 Da) were dynamic modifications. To determine the mutation conversion, proteotypic peptide, SAMPEGYVQER, was used. The signal was normalized with the injected peptide amount.

tRNA Sequencing and Data Analysis

~1 μg of total RNA was used to build tRNA sequencing libraries, following previously published MSR-seq protocol². Raw 100 bp paired-end sequencing reads were obtained from the Illumina NovaSeq platform. The data analysis also followed the MSR-seq data processing pipeline with minor customizations. Specifically, read 2 was processed and mapped to a curated reference which include all the human tRNA sequences as well as the 5 mctRNA sequences. Given the sequence similarities between mctRNAs and their cognate endogenous tRNAs, only mapped reads that are longer than 60 nt are used for abundance, charging and mutation analysis.

### RNA-Seq and Data Analysis

RNA-seq experiments were performed on 3 independent replicates from HEK293 cells overexpressing the mistranslation reporters with and without cognate mctRNAs. HEK293 cells were sorted on the FACSAria Fusion 5-18 cell sorter for mCherry+, i.e., successfully transfected, cell populations 48 h post-transfection. Total RNAs were extracted from sorted cells with TRIzol™ reagent (Invitrogen Cat. 15596026). 1 μg of total RNA per sample was used as input for RNA-seq library construct. Total RNA samples were polyA-selected to enrich mature mRNA species. All RNA libraries were multiplexed and sequenced on the Novaseq 6000 platform (Illumina) and each sample obtained ~120 million directional 100 bp pair-end (PE) reads. PE reads were mapped with STAR 2.7.10b using the reference human genome GRCh38.p10. Uniquely mapped reads were filtered and number of reads per gene for all genes was counted with featureCounts 2.0.1. Differential gene analysis was conducted using edgeR (version 3.40.2). For significantly up- or down-regulated genes (p<0.05 and absolute fold change>2), gene ontology analysis was conducted using clusterProfiler v4.6.2.

### Western Blot

Total protein samples were extracted from HEK293 cells 48 h post-transfection. To prevent CAPN3 autolytic activities, 15 mM EDTA and 1× protease inhibitor cocktail were added to all the buffers during sample preparation. In addition, samples were kept on ice until denaturation. 1-2 million transfected HEK293 cells were washed once with ice-cold 1×PBS and lysed with 30 μl CelLytic™ M buffer (Sigma-Aldrich Cat. C2978). Cell lysates were centrifuged at 17,000 g, 4° C. for 15 min to pellet cell debris. Supernatants were collected and incubated at 70° C. for 15 min upon adding 10 μl of 4× NuPAGE LDS sample buffer with 5% β-Mercaptoethanol. Denatured protein samples were loaded on 4-12% NuPAGE gels for gel electrophoresis. The NuPAGE gel, filter papers and the pre-wetted Immobilon®-P PVDF membrane (Sigma-Aldrich cat. IPVH00010) were incubated in transfer buffer (25 mM Tris-HCl, 192 mM Glycine, 10% methanol) for 10 min before transfer. Membrane transfer was conducted with the Trans-Blot Turbo Transfer System using the following settings: constant 25V; limit 1A; 20 min. The membrane was first blocked in the blocking buffer (5% nonfat dry milk (BIO-RAD Cat. 1706404) in 1×TBST buffer) for 1 hour at room temperature and then incubated with 1:1000 dilution of primary antibodies, anti-CAPN3 (Proteintech® Cat. 67366-1-Ig) and anti-GAPDH (Invitrogen Cat. MA5-15738), in the blocking buffer at 4° C. overnight. The membrane was then washed with 1×TBST 3 times, each time for 10 min. Next, the membrane was incubated with 1:10,000 dilution of the IRDye® 680RD Goat anti-Mouse IgG secondary antibody (LI-COR® Cat. 926-68070) in the blocking buffer for 1 hour at room temperature. The blotted membrane was then washed with 1×TBST 3 times, each time for 10 min, before being imaged under the Amersham Typhoon™ IR short channel.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Shevchenko, A., Wilm, M., Vorm, O. & Mann, M. Mass Spectrometric Sequencing of Proteins from Silver-Stained Polyacrylamide Gels. Anal Chem 68, 850-858 (1996).
Watkins, C. P., Zhang, W., Wylder, A. C., Katanski, C. D. & Pan, T. A multiplex platform for small RNA sequencing elucidates multifaceted tRNA stress response and translational regulation. Nat Commun 13, (2022).

What is claimed is:

1. A tRNA molecule covalently linked to a first amino acid, the tRNA molecule comprising an anticodon loop sequence capable of hybridizing with an mRNA sequence that encodes for a second amino acid that is different from the first amino acid.

2. The tRNA molecule of claim 1, wherein the first amino acid is arginine.

3. The tRNA molecule of claim 2, wherein the anticodon loop sequence is UGC, GCA, GUC, UUC, GAA, UCC, GUG, GAU, UUU, UAG, UAA, CAU, GUU, UGG, UUG, UCG, UGA, GCU, UGU, UAC, GUA, AGC, CGC, CUG, CUC, CCC, GCC, AAU, UAU, AAG, CAA, CAG, CUU, CGG, AGA, CGA, AGU, CGU, CCA, AAC, CAC, or AGG.

4. The tRNA molecule of claim 3, wherein the anticodon loop sequence is GUG, CUG, UUG, AGG, CGG, UGG, AAG, CAG, UAG, GCA, CCA, GCU, GCC, CCC, UCC. CUU, UUU, CGU, UGU, UAU, or CAU.

5. The tRNA molecule of claim 4, wherein the anticodon loop sequence is CUG, UUG, GUG, CCA, or GCA, from 5′ to 3′.

6. The tRNA molecule of claim 2, wherein the anticodon loop sequence is not ACG, CCG, CCU, UCG, GCG, or UCU, from 5′ to 3′.

7. The tRNA molecule of any of claims 2-6, wherein the mRNA sequence is not CGU, CGC, CGA, CGG, AGA, or AGG, from 5′ to 3′.

8. The tRNA molecule of any of claims 2-6, wherein the mRNA sequence is GCU, GCC, GCA, GCG, AAU, AAC, GAU, GAC, UGU, UGC, CAA, CAG, GAA, GAG, GGU, GGC, GGA, GGG, CAU, CAC, AUU, AUC, AUA, CUU, CUC, CUA, CUG, UUA, UUG, AAA, AAG, AUG, UUU, UUC, CCU, CCC, CCA, CCG, UCU, UCC, UCA, UCG, AGU, AGC, ACU, ACC, ACA, ACG, UGG, UAU, UAC, GUU, GUC, GUA, or GUG, from 5′ to 3′.

9. The tRNA molecule of claim 1, wherein the first amino acid is serine.

10. The tRNA molecule of claim 9, wherein the anticodon loop sequence is UGC, GCA, GUC, UUC, GAA, UCC, GUG, GAU, UUU, UAG, UAA, CAU, GUU, UGG, UUG, UCG, UGA, GCU, UGU, UAC, GUA, AGC, CGC, ACG, CCG, CCU, UCU, CUG, CUC, CCC, GCC, AAU, UAU, AAG, CAA, CAG, CUU, CGG, AGU, CGU, CCA, AAC, CAC, or AGG.

11. The tRNA molecule of claim 10, wherein the anticodon loop sequence is GAA, CAA, UAA, AGG, CGG, UGG, AGU, CGU, UGU, AGC, CGC, UGC, GUA, GCA, ACG, GUU, AAU, GCC, CCU, or UCU, from 5′ to 3′.

12. The tRNA molecule of claim 9, wherein the anticodon loop sequence is not AGA, CGA, GCU, UGA, ACU, or GGA, from 5′ to 3′.

13. The tRNA molecule of any of claims 9-12, wherein the mRNA sequence is not UCU, UCC, UCA, UCG, AGU, or AGC, from 5′ to 3′.

14. The tRNA molecule of any of claims 9-12, wherein the mRNA sequence is GCU, GCC, GCA, GCG, CGU, CGC, CGA, CGG, AGA, AGG, AAU, AAC, GAU, GAC, UGU, UGC, CAA, CAG, GAA, GAG, GGU, GGC, GGA, GGG, CAU, CAC, AUU, AUC, AUA, CUU, CUC, CUA, CUG, UUA, UUG, AAA, AAG, AUG, UUU, UUC, CCU, CCC, CCA, CCG, ACU, ACC, ACA, ACG, UGG, UAU, UAC, GUU, GUC, GUA, or GUG, from 5′ to 3′.

15. The tRNA molecule of any of claims 1-14, wherein the first amino acid is alanine, asparagine, aspartic acid, cysteine, glutamic acid, glutamine, glycine, histidine, hydroxyproline, isoleucine, leucine, lysine, methionine, phenylalanine, proline, threonine, tryptophan, tyrosine, or valine.

16. The tRNA molecule of any of claims 1-14, wherein the second amino acid is alanine, asparagine, aspartic acid, cysteine, glutamic acid, glutamine, glycine, histidine, hydroxyproline, isoleucine, leucine, lysine, methionine, phenylalanine, proline, threonine, tryptophan, tyrosine, or valine.

17. The tRNA molecule of any of claims 1-16, wherein the second amino acid is glutamine, histidine, tryptophan, or cysteine.

18. The tRNA molecule of any of claims 1-17, wherein the mRNA sequence is not UAA, UGA, or UAG.

19. The tRNA molecule of any of claims 1-18, wherein the tRNA molecule comprises a sequence having at most three substitutions relative to a mammalian tRNA molecule.

20. The tRNA molecule of claim 19, wherein the tRNA molecule comprises a sequence having at most three substitutions relative to a human tRNA molecule.

21. A nucleic acid comprising a sequence encoding for the tRNA of any of claims 1-20.

22. The nucleic acid of claim 21, wherein the sequence is one of SEQ ID NOs:1-144.

23. A vector comprising the nucleic acid of claim 21 or 22.

24. The vector of claim 23, wherein the vector is an AAV vector.

25. A cell comprising the tRNA molecule of any one of claims 1-20, the nucleic acid of any one of claims 21-22, and/or the vector of any one of claims 23-24.

26. The cell of claim 25, wherein the nucleic acid and/or vector is stably expressed.

27. A method for modifying a protein produced by a gene, the method comprising administering to a cell an effective amount of the tRNA of any of claims 1-20, the nucleic acid of claim 21 or 22, or the vector of claim 23 or 24.

28. A method for producing a wild type protein from a gene having a missense mutation, the method comprising administering to a cell an effective amount of the tRNA of any of claims 1-20, the nucleic acid of claim 21 or 22, or the vector of claim 23 or 24.

29. The method of claim 27 or 28, wherein the cell is a mammalian cell.

30. The method of claim 29, wherein the cell is a human cell.

31. A method for treating or preventing a genetic disease in a subject, the method comprising administering to the subject a therapeutically effective amount of the tRNA of any of claims 1-20, the nucleic acid of claim 21 or 22, or the vector of claim 23 or 24.

32. The method of claim 31, wherein the genetic disease is characterized by the presence of a single nucleotide variation (SNV) resulting in a missense mutation in a gene.

33. The method of claim 32, wherein the gene is ABCD1, GLA, GBA, GALC, ARSA, SGSH, HGSNAT, IDS, OTC, DHCR7, or HEXA.

34. The method of any of claims 31-33, wherein the gene is a gene of Table 2.

35. The method of any of claims 31-34, wherein the SNV is a SNV of Table 2.

36. The method of any of claims 31-35, wherein the genetic disease is a recessive disease.

37. The method of any of claims 31-36, wherein the genetic disease is Adrenoleukodystrophy, Fabry disease, Gaucher disease type I, Metachromatic leukodystrophy, Mucopolysaccharidosis, Ornithine transcarbamylase deficiency, Smith-Lemli-Opitz syndrome, Tay-Sachs disease, Niemann-Pick disease, or Very long chain acyl-CoA dehydrogenase deficiency.
